(19) United States
(12) Patent Application Publication
(10) Pub. No.: US 2025/0034642 A1
(43) Pub. Date: Jan. 30, 2025

(54) DNA METHYLATION BIOMARKERS FOR RHEUMATOID ARTHRITIS

(71) Applicant: WASHINGTON STATE UNIVERSITY, Pullman, WA (US)

(72) Inventor: Michael K. SKINNER, Pullman, WA (US)

(21) Appl. No.: 18/715,368

(22) PCT Filed: Dec. 2, 2022
(86) PCT No.: PCT/US2022/080837

§ 371 Date: May 31, 2024

Related U.S. Application Data
(60) Provisional application No. 63/285,829, filed on Dec. 3, 2021.

(51) Int. Cl. C12Q 1/6883; C12Q 1/6874

(52) U.S. Cl. CPC C12Q 1/6883; C12Q 1/6874; C12Q 2600/154

(57) ABSTRACT

The present disclosure relates to methods of analyzing the methylation level of differential DNA methylation regions (DMRs) obtained from buccal and blood monocyte cells. Also disclosed herein are methods of analyzing methylation level of DMRs for identification and preventative treatments of rheumatoid arthritis in human subjects.

### CROSS REFERENCE TO RELATED APPLICATIONS

This application claims the benefit under 35 USC § 119(e) of U.S. Provisional Application Ser. No. 63/285,829, filed on Dec. 3, 2021, the entire disclosure of which is incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

The present disclosure relates to methods of analyzing the methylation level of differential DNA methylation regions (DMRs) obtained from buccal and blood monocyte cells. More particularly, the present disclosure relates to methods of analyzing methylation level of DMRs for identification and preventative treatment of rheumatoid arthritis in human subjects.

### SUMMARY

Rheumatoid arthritis (RA) is a chronic autoimmune inflammatory disease with a worldwide prevalence of 0.5-1.0%. The frequency of RA is predominant in females and has increased in the past few decades, and is proportionally higher in North American populations. RA is characterized by synovial hyperplasia and joint destruction. RA impacts both African American and Caucasian populations, with higher comorbidity prevalence in African American populations.

The risk of developing RA is influenced by environment and lifestyle factors, including smoking, nutrition, and alcohol consumption. Environmental toxicant exposures have been shown to be involved in the etiology of rheumatoid arthritis.

Genetics has been assumed to be a major factor in rheumatoid arthritis etiology. A number of genome-wide association studies (GWAS) have been performed and identified hundreds of single-nucleotide polymorphisms (SNPs) that are associated with RA. Potential secondary gene associations suggest genetics can potentially explain 30% of familial disease cases. However, an alternate molecular process involving epigenetics is now assumed to be equally important and a significant factor in the etiology of RA. Since environmental factors generally cannot directly change DNA sequence to alter genetic processes, the environmental impacts observed on RA etiology involve epigenetics. Epigenetics provides the molecular process for environmental factors such as nutrition and toxicants to impact genetics. Therefore, an integration of environment, epigenetics and genetics is now thought to be involved in the etiology and progression of rheumatoid arthritis.

Epigenetics is generally understood to involve molecular factors and processes around DNA that regulate genome activity independent of DNA sequence, and are mitotically stable. Epigenetic factors include DNA methylation, histone modifications, non-coding RNA (ncRNA), chromatin structure and RNA methylation. Although all these processes will be involved in RA, DNA methylation at 5-methylcytosine has been the primary epigenetic process investigated in rheumatoid arthritis. Epigenome-wide association studies (EWAS) have been used to identify specific immune gene associations, as have genome-wide investigations.

Rheumatoid arthritis is generally diagnosed today after the onset of disease when clinical characteristics develop. Therefore, most diagnoses of RA is based on symptomatic parameters and therapeutic intervention of RA is limited to treating the symptoms of a disease that has already progressed. Although several RA biomarkers and diagnostics have been developed, none are focused on early-stage biomarkers that can be used prior to the onset of clinical symptoms. Thus, there exists a need for new methods and mechanisms for developing early-stage RA biomarkers and diagnostics to provide effective preventative treatment of RA prior to disease onset.

Accordingly, the present disclosure provides methods and kits that utilize the concepts of epigenetics to meet the need for early-stage RA biomarkers and diagnostics. Importantly, the methods and kits of the present disclosure can provide options for preventative treatment of RA by identifying susceptible individuals at risk for developing RA prior to the onset of disease symptoms. Furthermore, the methods and kits of the present disclosure can provide important information for administration of preventative therapeutics to be administered to susceptible individuals. For example, such preventative therapeutics could be used to delay or to even prevent the onset of RA disease from developing.

The methods and kits described herein have several desirable features. First, the methods of the present disclosure can utilize buccal cells that are easily obtained from subjects via a cheek swab and have a high level of purity. Furthermore, although some epigenetic studies have investigated DNA from cells obtained from blood, it is known that blood provides a mixture of cells that contains over 20 different cell types. Since each individual cell type is known to have a unique epigenome to give the cell type its specificity, a mixed cell type analysis can be misleading and reflect changes in cell populations instead of epigenetic change. Accordingly, the present disclosure also contemplates using purified blood monocytes to obtain DNA for epigenetic analysis.

Finally, the present disclosure provides the ability to identify RA susceptibility in patients without the onset of RA. The use of the described RA susceptibility biomarkers permits the initiation of preventative therapeutic intervention to delay or prevent the onset of RA in the most susceptible patients.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIGS. 1A-1F show rheumatoid arthritis (RA) DMR identifications. FIG. 1A: Caucasian control versus RA buccal cell DMR analysis. FIG. 1B: Caucasian control versus RA monocyte cell DMR analysis. FIG. 1C: African American (AA) control versus RA buccal cell DMR analysis. FIG. 1D: Combined Caucasian (CC) and African American (AA) control versus RA buccal cell DMR analysis. The number of DMRs found using different p-value cutoff thresholds. The all window column shows all DMRs. The multiple window column shows the number of DMRs containing at least two adjacent significant windows and the number of DMRs with each specific number of significant windows at a p-value threshold of p<1e-04. FIG. 1E: Venn diagram overlap of the RA DMRs at p<1e-04 for the Caucasian monocyte, buccal and AA buccal, and combined CC and AA (All) buccal. FIG. 1F: Extended overlap with a comparison of RA DMRs in the different comparison at p<1e-04 versus horizontal p<0.05 for the different comparisons. The overlapping DMR numbers and percent (%) of the total is presented. The highlighted overlaps for All buccal and overlaps indicated.

FIGS. 2A-2D show RA DMR chromosomal locations and principal component analysis (PCA). The DMR locations on the individual chromosomes are identified. All DMRs at a p-value threshold of p<1e-04 are shown with arrowheads and clusters of DMRs with the black boxes. FIG. 2A: Caucasian control versus RA buccal DMRs. FIG. 2B: Caucasian control versus RA monocyte DMRs. FIG. 2C: AA control versus RA buccal DMRs. FIG. 2D: Combined CC and AA for All buccal control versus RA buccal DMRs. All DMRs at a p-value threshold of p<1e-04.

FIGS. 3A-3D show control versus RA DMR principal component analysis (PCA). PCA analysis for DMRs at p<1e-04. FIG. 3A: Caucasian control versus RA. FIG. 3B: Caucasian monocyte DMR PCA. FIG. 3C: AA control versus AA RA buccal DMR PCA. FIG. 3D: CC and AA all buccal control versus all RA buccal DMR PCA.

FIGS. 4A-4B show RA DMR associated gene categories and pathways. FIG. 4A: DMR associated gene categories. DMR numbers at a p-value threshold p<1e-04 are shown. The comparison DMR key is inset. FIG. 4B: DMR associated gene pathways. The pathways common for two or more comparisons are presented. Number in bracket is number of DMR associated genes in pathway

FIGS. 5A-5B show DMR associated genes from the present disclosure compared to genes associated with arthritis in the published literature using Pathway Studio software (Elsevier, Inc.). Those in common are depicted. FIG. 5A: African American (AA) buccal cell RA DMR associated gene disease correlations. FIG. 5B: Caucasian buccal DMR associated gene disease correlations.

FIGS. 6A-6B show DMR associated genes from the present disclosure compared to genes associated with arthritis in the published literature using Pathway Studio software (Elsevier, Inc.). Those in common are depicted. FIG. 6A: Combined CC and AA all buccal RA DMR associated gene disease correlations. FIG. 6B: Caucasian monocyte RA DMR associated gene disease correlations. The gene function symbol index are inset.

FIG. 7 shows DMR associated RA genes were correlated with known RA cell processes in the published literature using Pathway Studio software (Elsevier, Inc.). The DMR associated genes from all comparisons were linked to the RA cell processes.

FIGS. 8A-8H show DMR CpG density for CpG/100 bp and length (bp) are presented. FIG. 8A: Caucasian control versus RA buccal DMRs. FIG. 8B: Caucasian control versus RA buccal DMRs. FIG. 8C: Caucasian control versus RA monocyte DMRs. FIG. 8D: Caucasian control versus RA monocyte DMRs. FIG. 8E: AA control versus AA RA buccal DMRs. FIG. 8F: A control versus AA RA monocyte DMRs. FIG. 8G: AA and CC buccal combined control and AA and CC buccal combined RA DMRs. FIG. 8H: AA and CC all buccal combined controls versus AA and CC combined RA buccal DMRs.

FIG. 9 shows Table 1, which provides a DMR list comprising Caucasian control versus RA buccal (p<1e-04). The table includes DMR name, chromosomal location, DMR start and stop nucleotide number for the chromosome, length (bp) of the DMR, number of 1 kb significant windows, minimum p-value, CpG number (number per DMR) and density (number per 100 base pair), DMR maximum, log-fold change (maxLFC) (+ increase DNA methylation and (−) decrease DNA methylation), and gene association within 10 kb and gene functional categories. DMR statistics for EdgeR minimum p-value and FDR minimum p-value are presented. The Accession number is PMID 34893669 and the GEO accession number is GSE 186179. In some instances an increase in DNA methylation can comprise hypermethylation of a DMR. In some instances a decrease in DNA methylation can comprise hypomethylation of a DMR.

FIG. 10 shows Table 2, which provides a DMR list comprising Caucasian control versus RA monocyte (p<1e-04). The table includes DMR name, chromosomal location, DMR start and stop nucleotide number for the chromosome, length (bp) of the DMR, number of 1 kb significant windows, minimum p-value, CpG number (number per DMR) and density (number per 100 base pair), DMR maximum, log-fold change (maxLFC) (+ increase DNA methylation and (−) decrease DNA methylation), and gene association within 10 kb and gene functional categories. DMR statistics for EdgeR minimum p-value and FDR minimum p-value are presented. The Accession number is PMID 34893669 and the GEO accession number is GSE 186179. In some instances an increase in DNA methylation can comprise hypermethylation of a DMR. In some instances a decrease in DNA methylation can comprise hypomethylation of a DMR.

FIG. 11 shows Table 3, which provides a DMR list comprising African American control versus AA RA (p<1e-04). The table includes DMR name, chromosomal location, DMR start and stop nucleotide number for the chromosome, length (bp) of the DMR, number of 1 kb significant windows, minimum p-value, CpG number (number per DMR) and density (number per 100 base pair), DMR maximum, log-fold change (maxLFC) (+ increase DNA methylation and (−) decrease DNA methylation), and gene association within 10 kb and gene functional categories. DMR statistics for EdgeR minimum p-value and FDR minimum p-value are presented. The Accession number is PMID 34893669 and the GEO accession number is GSE 186179. In some instances an increase in DNA methylation can comprise hypermethylation of a DMR. In some instances a decrease in DNA methylation can comprise hypomethylation of a DMR.

FIG. 12 shows Table 4, which provides a DMR list comprising a combination of African American and Caucasian control versus combined African American and Caucasian (all buccal). The table includes DMR name, chromosomal location, DMR start and stop nucleotide number for the chromosome, length (bp) of the DMR, number of 1 kb significant windows, minimum p-value, CpG number (number per DMR) and density (number per 100 base pair), DMR maximum, log-fold change (maxLFC) (+ increase DNA methylation and (−) decrease DNA methylation), and gene association within 10 kb and gene functional categories. DMR statistics for EdgeR minimum p-value and FDR minimum p-value are presented. The Accession number is PMID 34893669 and the GEO accession number is GSE 186179. In some instances an increase in DNA methylation can comprise hypermethylation of a DMR. In some instances a decrease in DNA methylation can comprise hypomethylation of a DMR.

FIG. 13 shows Table 5, which provides DMR associated RA gene cell processes. The table shows the cell process name, total number neighbors, gene set seed, overlapping DMR associated genes, percent overlap, total list overlapping genes, Jaccard similarity, and p-value statistic for the overlapping gene set are presented.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless otherwise indicated, open terms for example “contain,” “containing,” “include,” “including,” and the like mean comprising.

The singular forms “a”, “an”, and “the” can be used herein to include plural references unless the context clearly dictates otherwise. For example, the term “a sample” includes a plurality of samples, including mixtures thereof.

As used herein, the term “about” or “approximately” a number can refer to that number plus or minus 10% of that number. In some cases, about or approximately can refer to that number plus or minus 5% of that number. The term about or approximately a range can refer to that range minus 10% of its lowest value and plus 10% of its greatest value. In some cases, the term about or approximately a range can refer to that range minus 5% of its lowest value and plus 5% of its greatest value. Alternatively, particularly with respect to biological systems or processes, the term can mean within an order of magnitude, within 5-fold, or within 2-fold, of a value. Where particular values or values of a range are described in the application and claims, unless otherwise stated the term “about” meaning within an acceptable error range for the particular value should be assumed.

The term “substantially” or “essentially” can refer to a qualitative condition that exhibits an entire or nearly total range or degree of a feature or characteristic of interest. In some cases, substantially can refer to at least about: 70%, 75%, 80%, 85%, 90%, 91%, 92%, 93%, 94%, 95%, 96%, 97%, 98%, 99% or 100% of the total range or degree of a feature or characteristic of interest.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 5 should be considered to have specifically disclosed subranges such as from 1 to 2, from 1 to 3, from 1 to 4, from 2 to 4, from 3 to 5, etc., as well as individual numbers within that range, for example, 1, 2, 3, 4, and 5. This applies regardless of the breadth of the range.

The terms “determining”, “measuring”, “evaluating”, “assessing,” “assaying,” and “analyzing” are often used interchangeably herein to refer to forms of measurement and include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The terms “subject,” “individual,” or “patient” are often used interchangeably herein. A “subject” can be a biological entity. The biological entity can be an animal, a plant, or a microorganism. The subject can be tissues, cells and their progeny of a biological entity obtained in vivo or cultured in vitro. The subject can be a mammal. The mammal can be a human. The subject may be a child, an infant, or an adult. In some cases, the human is a male. The subject can be a male human of reproductive age (e.g., older than 10 years of age). The subject can be about 1 day old to about 18 years old. In some cases, the subject can be about 1 day old to about 1 year old. In some cases, the subject can be older than 18 years of age. In some cases, the subject can be older than about 10 years, 30 years, 40 years, 50 years, 60 years, 70 year, 80 years or 90 years. The subject can be about 1, 5, 10, 15, 20, 25, 30, 35, 40, 45, 50, 55, 60, 65, 70, 75, 80, 85, 90, 95, 100, 105, 110, 115, 120, 125 or 130 years old. In some cases, the subject can be older than 60 or 65 years of age. The subject may be diagnosed or suspected of being at high risk for a disease. In some cases, the subject may not be necessarily diagnosed or suspected of being at high risk for the disease.

The term “in vitro” can be used to describe an event that takes place contained in a container for holding laboratory reagent such that it can be separated from the living biological source organism from which the material may be obtained. In vitro assays can encompass cell-based assays in which cells alive or dead are employed. In vitro assays can also encompass a cell-free assay in which no intact cells are employed.

The term “in vivo” can be used to describe an event that takes place in a subject's body.

The term “ex vivo” can be used to describe an event that takes place outside of a subject's body. An “ex vivo” assay may not be performed on a subject. Rather, it can be performed upon a sample separate from a subject. An example of an “ex vivo” assay performed on a sample can be an “in vitro” assay.

As used herein, the terms “treatment” or “treating” refers to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of one or more symptoms or of an underlying disorder being treated. For example, a therapeutic benefit can comprise reducing the pain from rheumatoid arthritis. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “effective amount” or “therapeutically effective amount” of a drug used to treat a disease can be an amount that can reduce the severity of a disease, reduce the severity of one or more symptoms associated with the disease or its treatment, or delay the onset of more serious symptoms or a more serious disease that can occur with some frequency following the treated condition. An “effective amount” may be determined empirically and in a routine manner, in relation to the stated purpose. Any treatment described herein can be administered in an effective amount or therapeutically effective amount.

As used herein, the term “unit dose” or “dosage form” can be used interchangeably and can be meant to refer to pharmaceutical drug products in the form in which they are marketed for use, with a specific mixture of active ingredients and inactive components or excipients, in a particular configuration, and apportioned into a particular dose to be delivered. The term “unit dose” can also sometimes refer to the particles comprising a pharmaceutical composition or therapy, and to any mixtures involved. Types of unit doses may vary with the route of administration for drug delivery, and the substance(s) being delivered. A solid unit dose can be the solid form of a dose of a chemical compound used as a pharmaceutically acceptable drug or medication intended for administration or consumption. Any treatment described herein can be in a unit dose.

As used herein, “pharmaceutically acceptable salt” can refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness. Any treatment described herein also comprises pharmaceutically acceptable salts of said treatment.

In some cases, a reference level is a reference methylation level. In some cases, a reference level is a control level of methylation. In some cases, a reference level of methylation can be a level of methylation from any of the DMRs in Table 1, Table 2, Table 3, Table 4, or any combination thereof. In some cases, a reference level of methylation can be a level of methylation from a plurality of DMRs in Table 1, Table 2, Table 3, Table 4, or any combination thereof. In some cases, a control level of methylation can be obtained from a diseased individual or a group of diseased individuals such as a group of individuals with rheumatoid arthritis. In some cases, a control level of methylation can be obtained from a healthy individual or a group of healthy individuals without a disease or condition. In some cases, a control level of methylation is from a known cell type, such as a somatic cell.

As used herein, the term “reference sequence”, can refer to a known nucleotide sequence, e.g. a chromosomal region whose sequence is deposited at NCBI's Genbank database or other databases. A reference sequence can be a wild type sequence.

The term “nucleic acid” and “polynucleotide” can be used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., peptide nucleic acid (PNA)) which can hybridize with naturally occurring nucleic acids in a sequence specific manner analogous to that of two naturally occurring nucleic acids, e.g., can participate in Watson-Crick base pairing interactions. Naturally-occurring nucleotides can include guanine, cytosine, adenine, uracil and thymine (G, C, A, U and T, respectively). In some cases, a nucleic acid can be single stranded. In some cases, a nucleic acid can be double stranded. In some cases, a nucleic acid can comprise a ribonucleic acid (RNA), deoxyribonucleic acid (DNA), or both. In some cases, a polynucleotide may have a modified base.

The section headings used herein are for organizational purposes only and are not to be construed as limiting the subject matter described.

In an illustrative aspect, a first method is provided. The method comprises obtaining a sample of a somatic cell from a human subject; isolating deoxyribonucleic acid (DNA) from the sample, thereby obtaining isolated DNA; detecting and determining a methylation level of a differential DNA methylation region (DMR) comprised in the isolated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and wherein a plurality of distinct DMRs are detected and compared, In an embodiment, the plurality comprises at least 10 distinct DMRs.

In an embodiment, the somatic cell comprises a buccal cell. In an embodiment, the somatic cell consists essentially of a buccal cell. In an embodiment, the somatic cell consists of a buccal cell.

In an embodiment, the somatic cell comprises a blood monocyte cell. In an embodiment, the blood monocyte cell is a purified blood monocyte cell. In an embodiment, the somatic cell consists essentially of a blood monocyte cell. In an embodiment, the blood monocyte cell is a purified blood monocyte cell. In an embodiment, the somatic cell consists of a blood monocyte cell. In an embodiment, the blood monocyte cell is a purified blood monocyte cell. Methods for purifying blood monocyte cells are well known in the art to the skilled artisan. In some cases, a cell in a sample can be the same cell type. In some cases, a cell in a sample can be of different cell types. For example, a cell in a sample can be a buccal cell and a blood cell.

In some embodiments, a sample can comprise more than one cell such as a somatic cell. In some cases, a sample can comprise about 10 cells, 100 cells, 1000 cells, 10000 cells, 100,000 cells, 1,000,000 cells, 10,000,000 cells, 100,000,000 cells or more than 100,000,000 cells. In some cases, a sample can comprise about: 1 cell to about 1×10\hat{ }11 cells, 1 cell to about 10 cells, 1 cell to about 100 cells, 10 cells to about 1000 cells, 100 cells to about 10,000 cells, 1000 cells to about 100,000 cells, 10,000 cells to about 1,000,000 cells, 10×10\hat{ }5 cells to about 10×10\hat{ }7 cells, 10×10\hat{ }6 cells to about 10×10\hat{ }8 cells, 10×10\hat{ }7 cells to about 10×10\hat{ }10 cells, or about 10×10\hat{ }8 cells to about 10×10\hat{ }11 cells.

In an embodiment, the somatic cell comprises a muscle cell, a blood cell, a skin cell, a nerve cell, or any combination thereof. In an embodiment, the somatic cell comprises an immune cell. In an embodiment, the somatic cell comprises a muscle cell. In an embodiment, the somatic cell comprises a blood cell. In an embodiment, the somatic cell comprises a skin cell. In an embodiment, the somatic cell comprises a nerve cell.

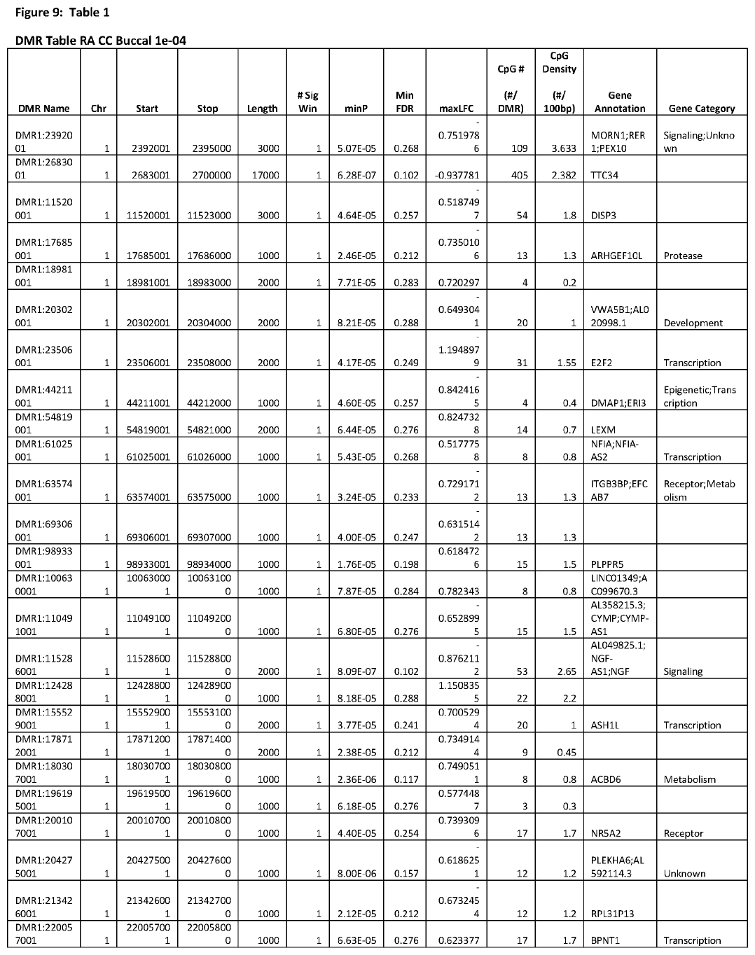

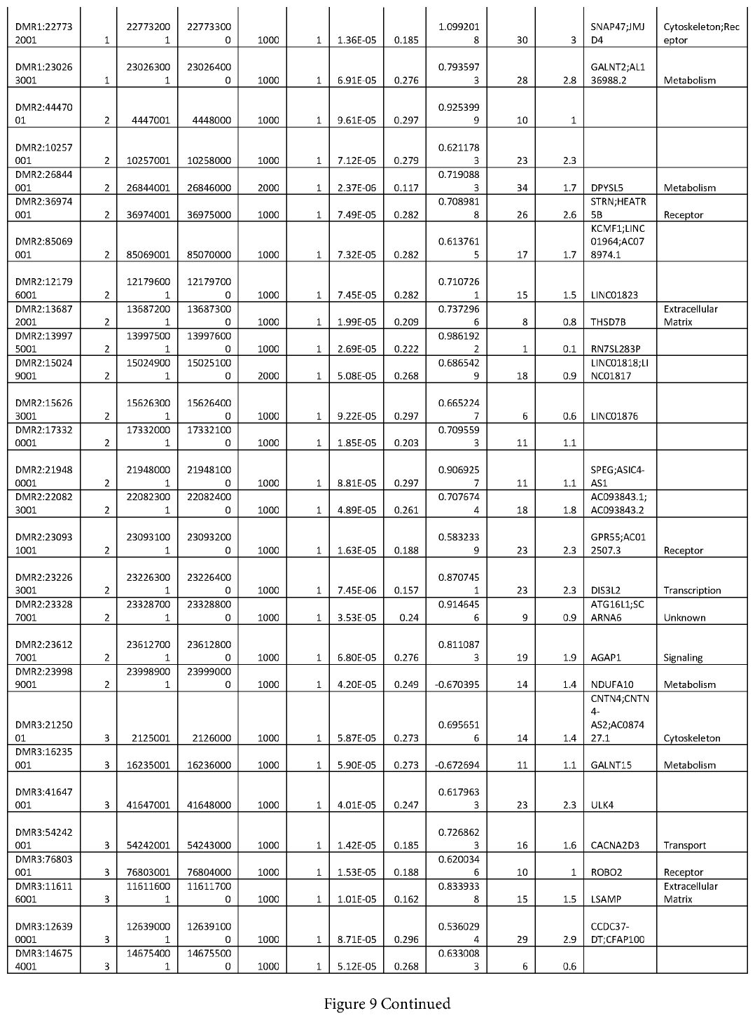

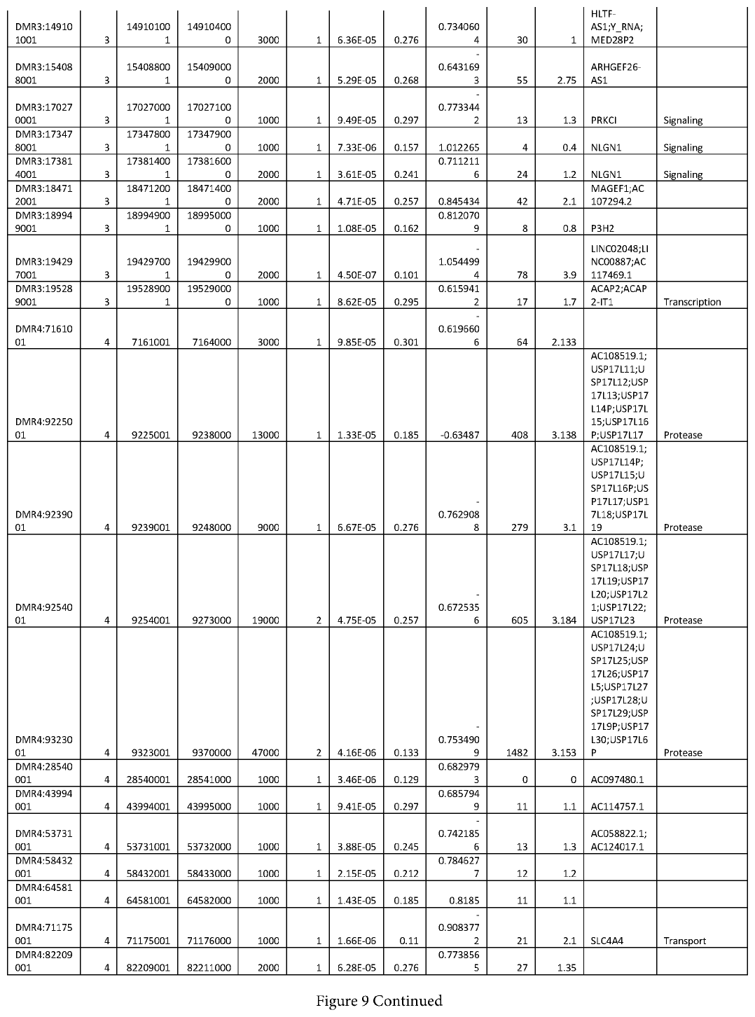

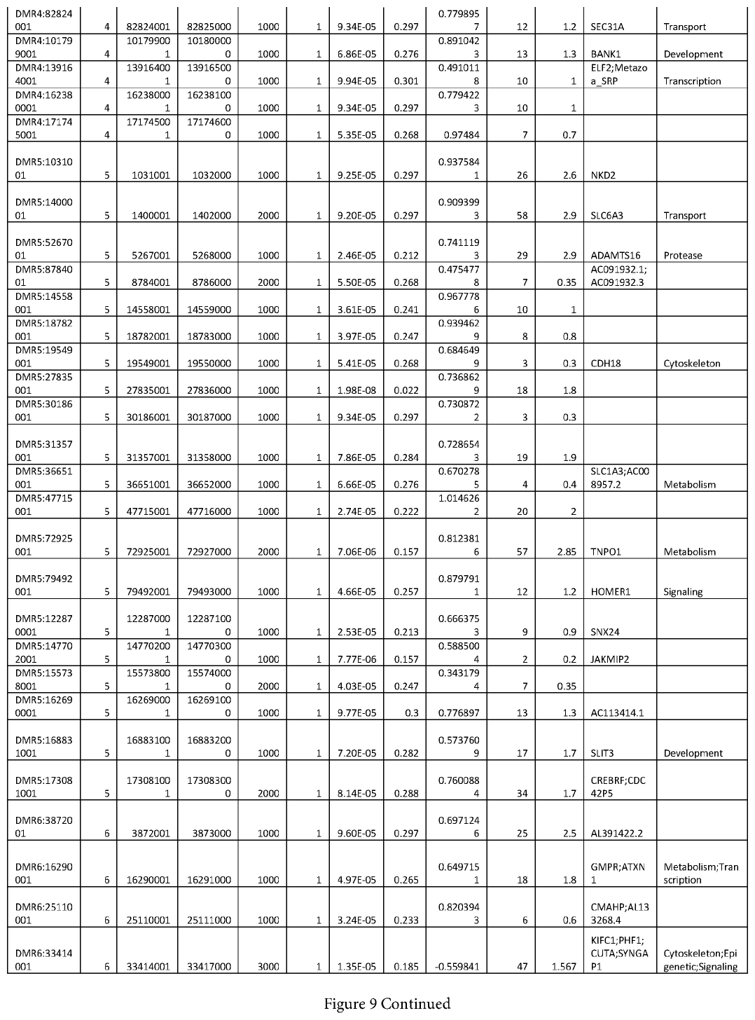

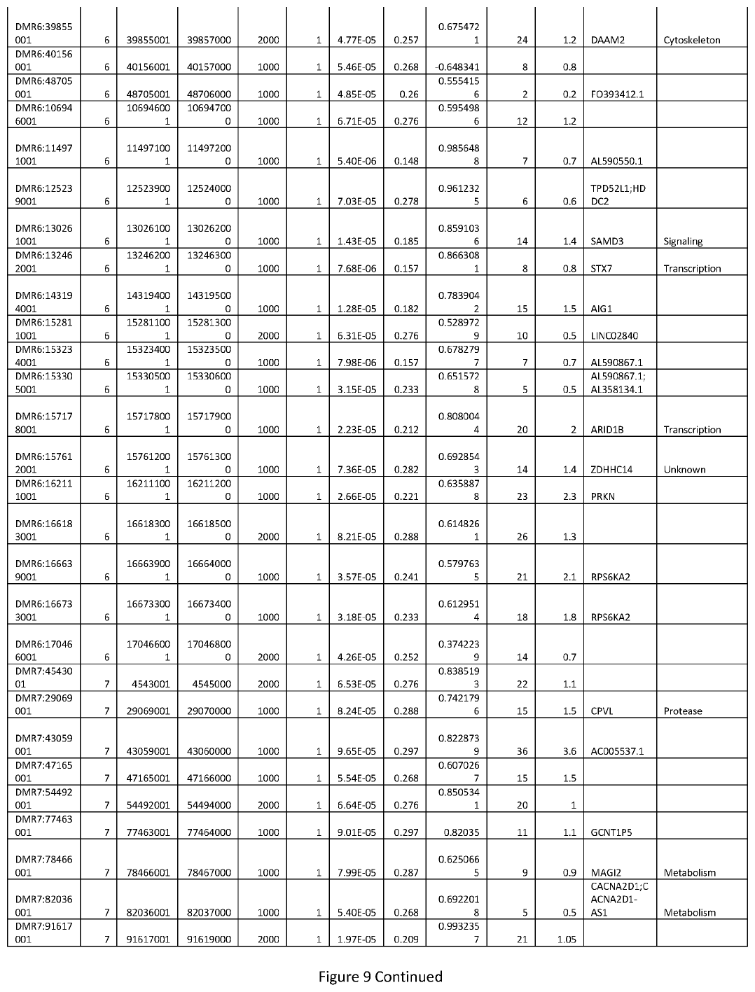

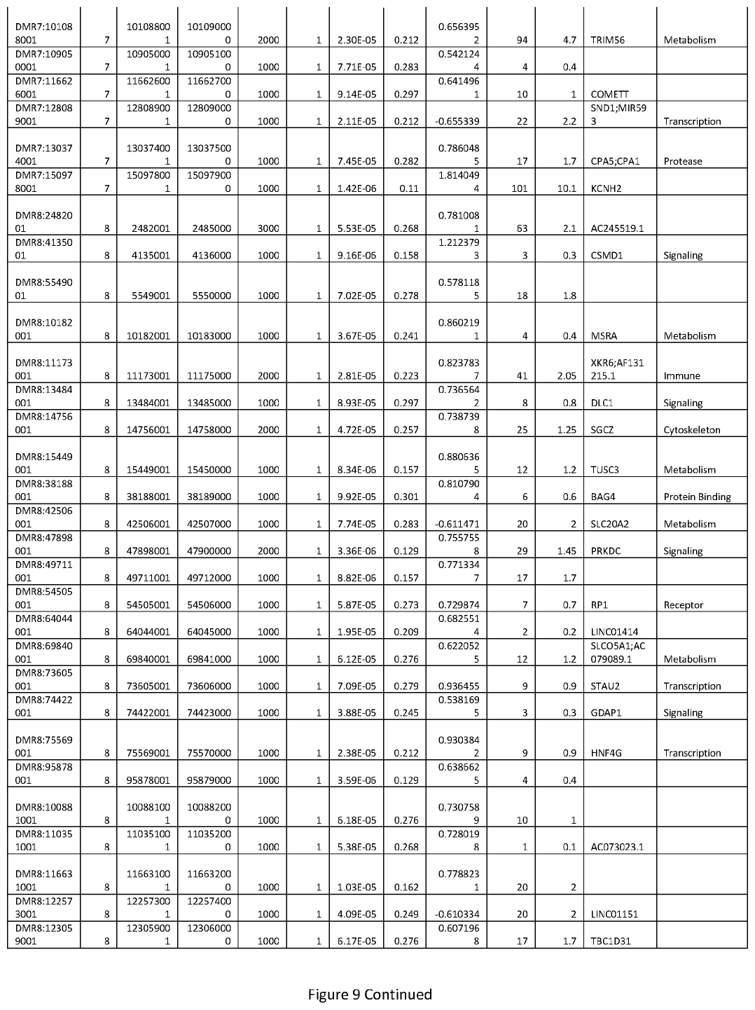

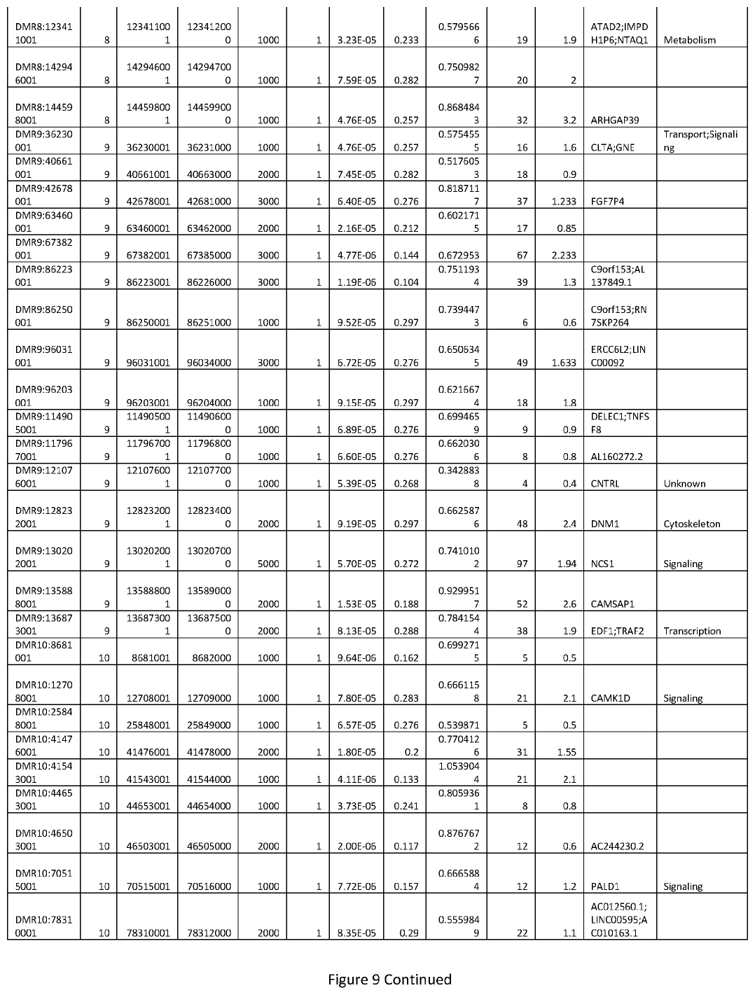

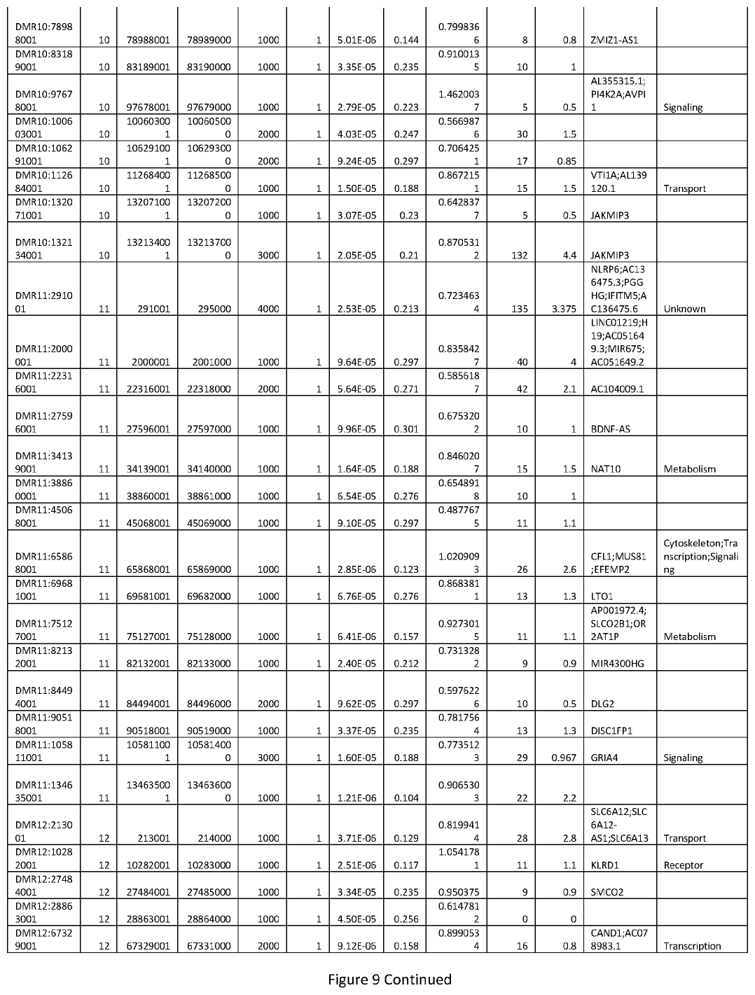

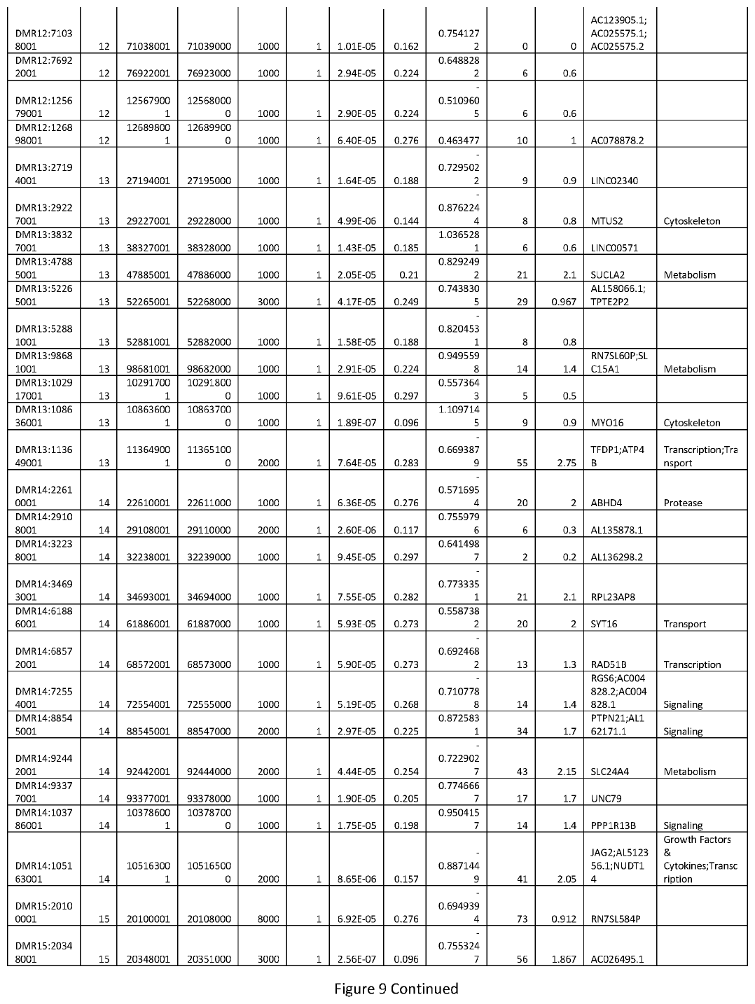

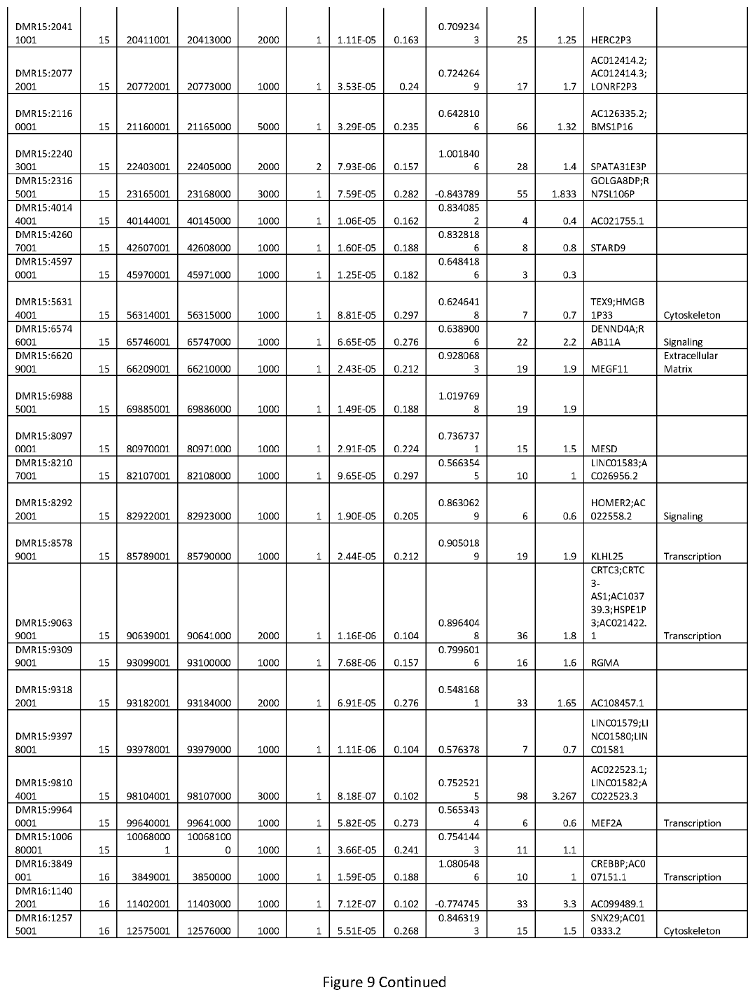

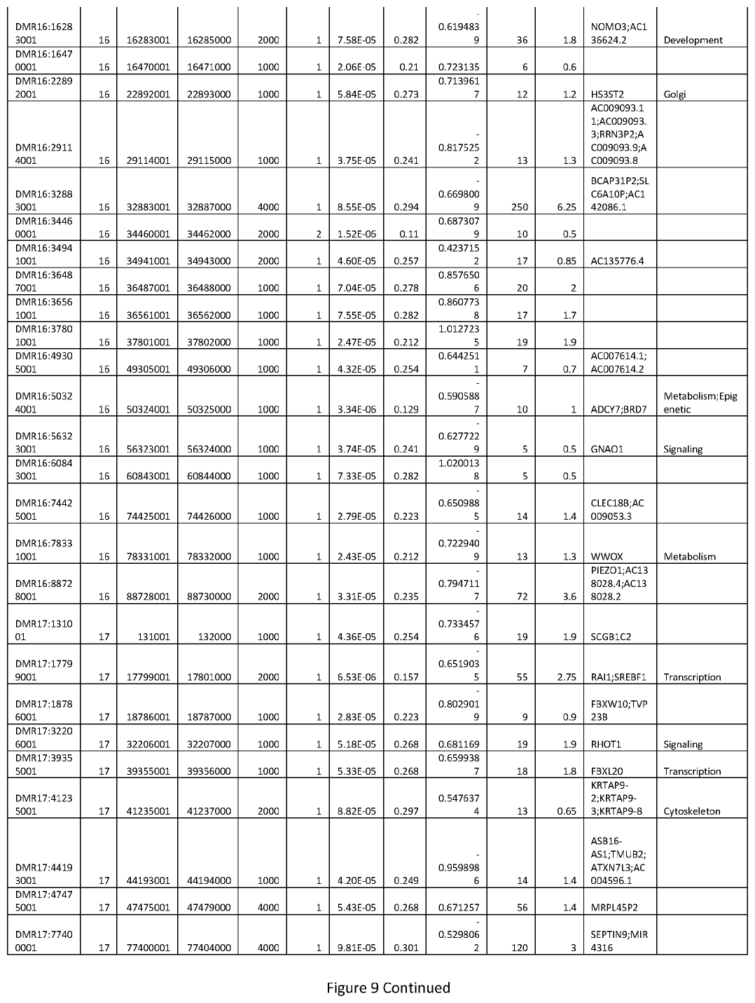

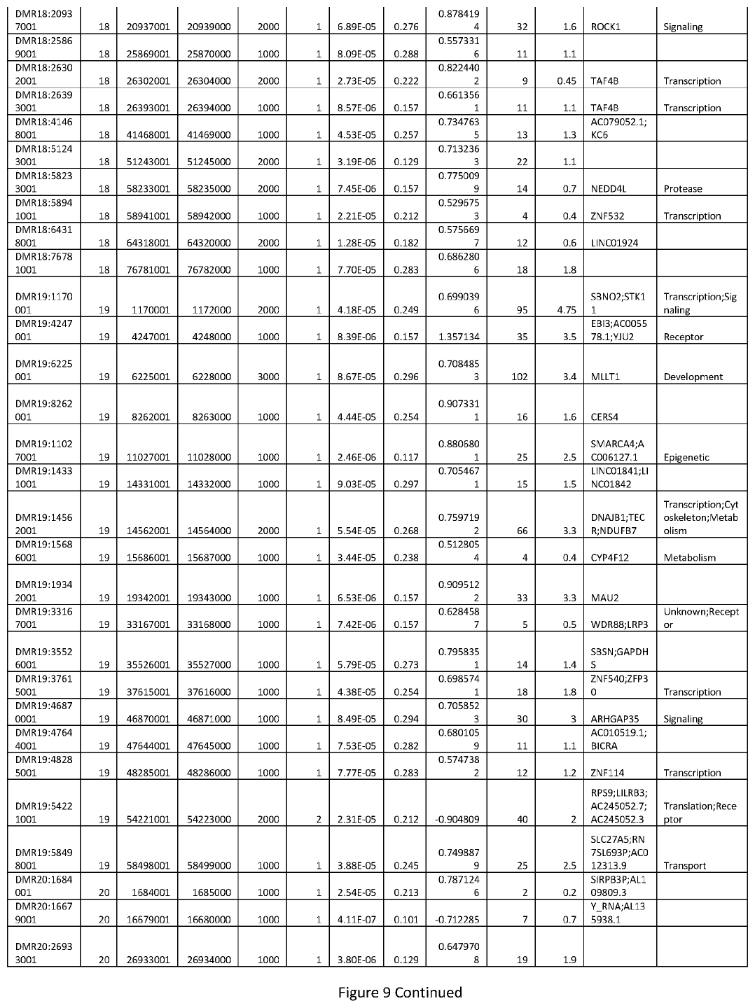

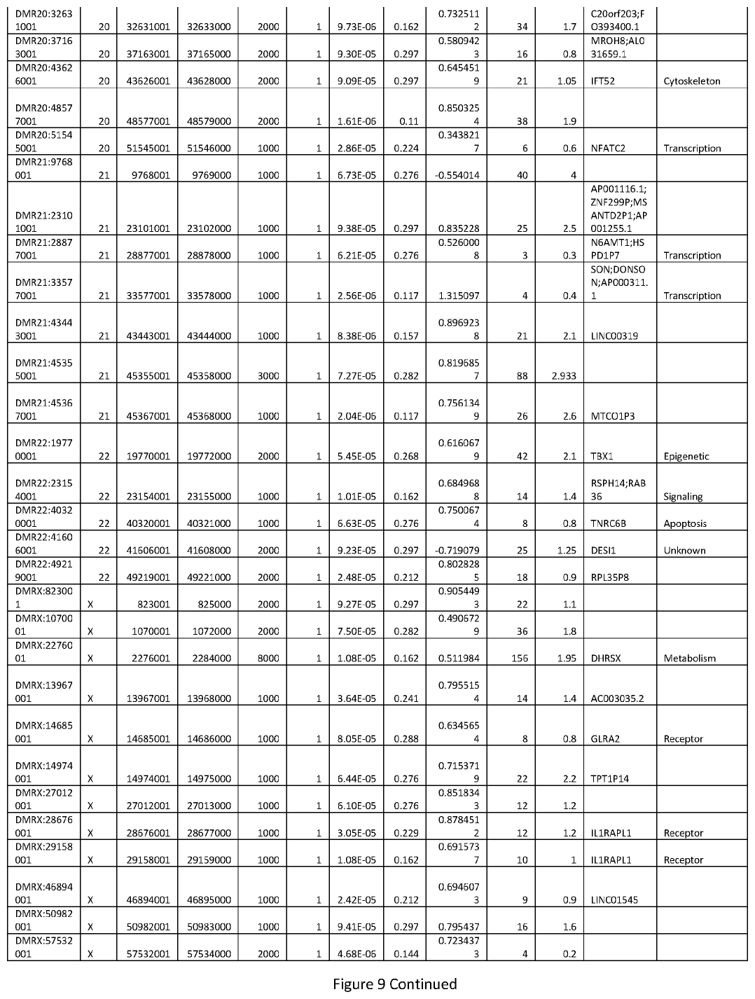

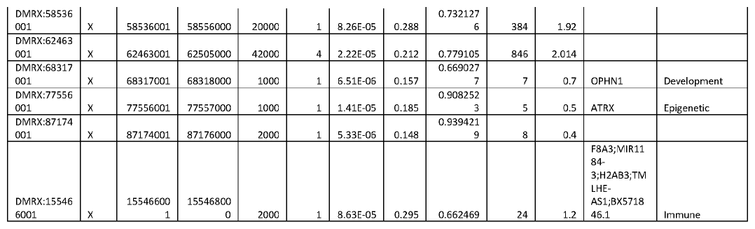

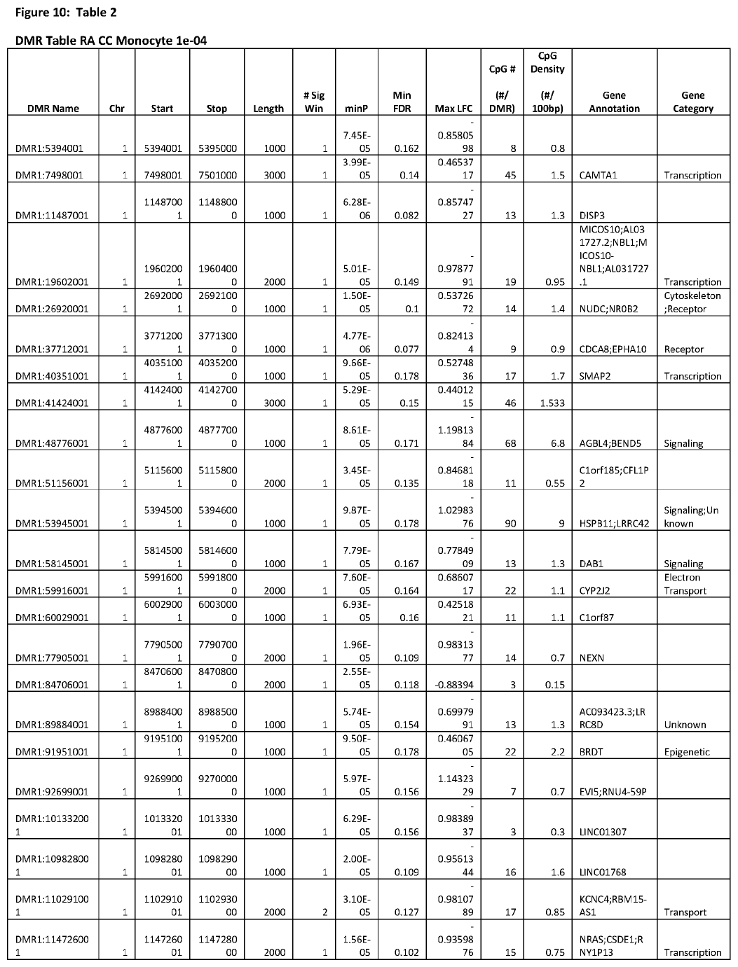

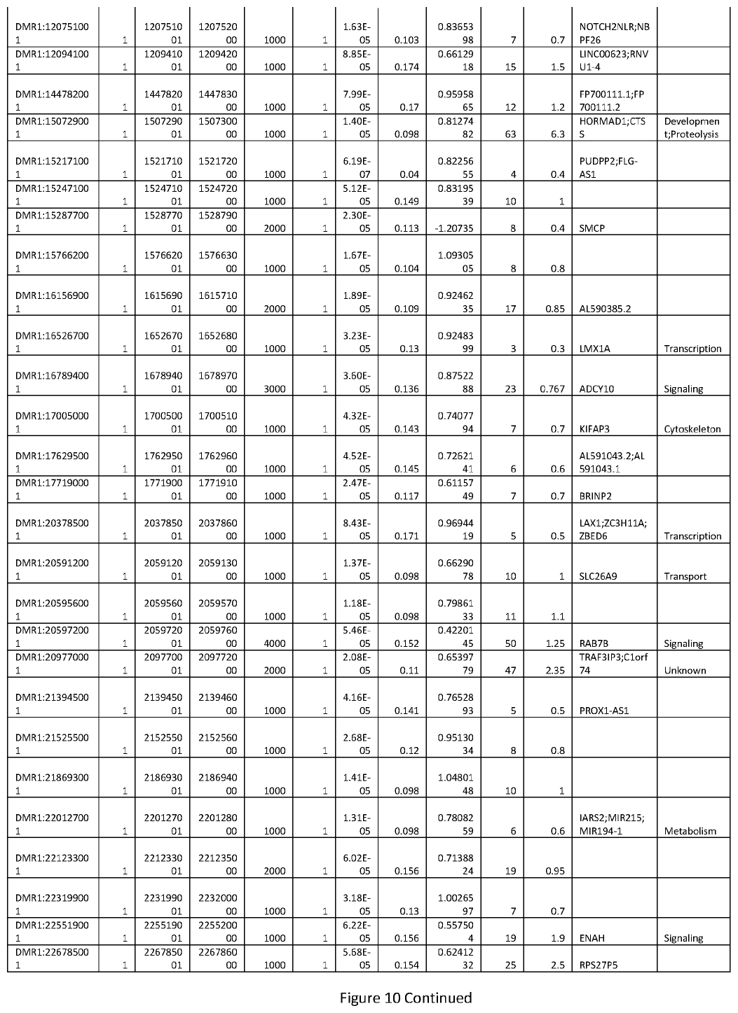

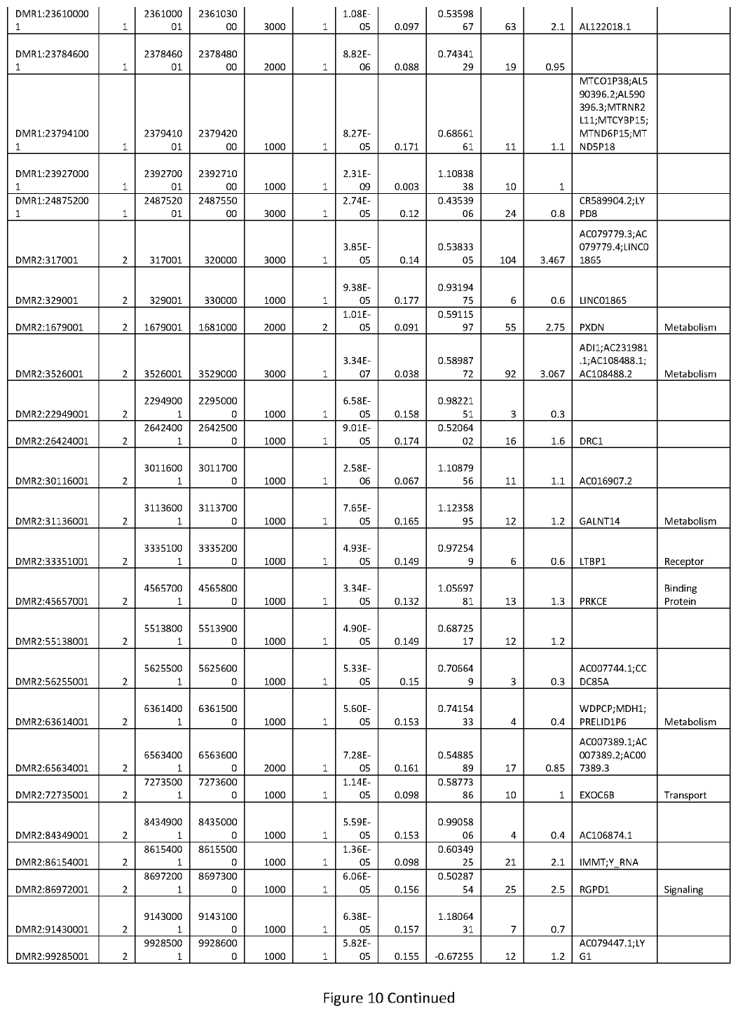

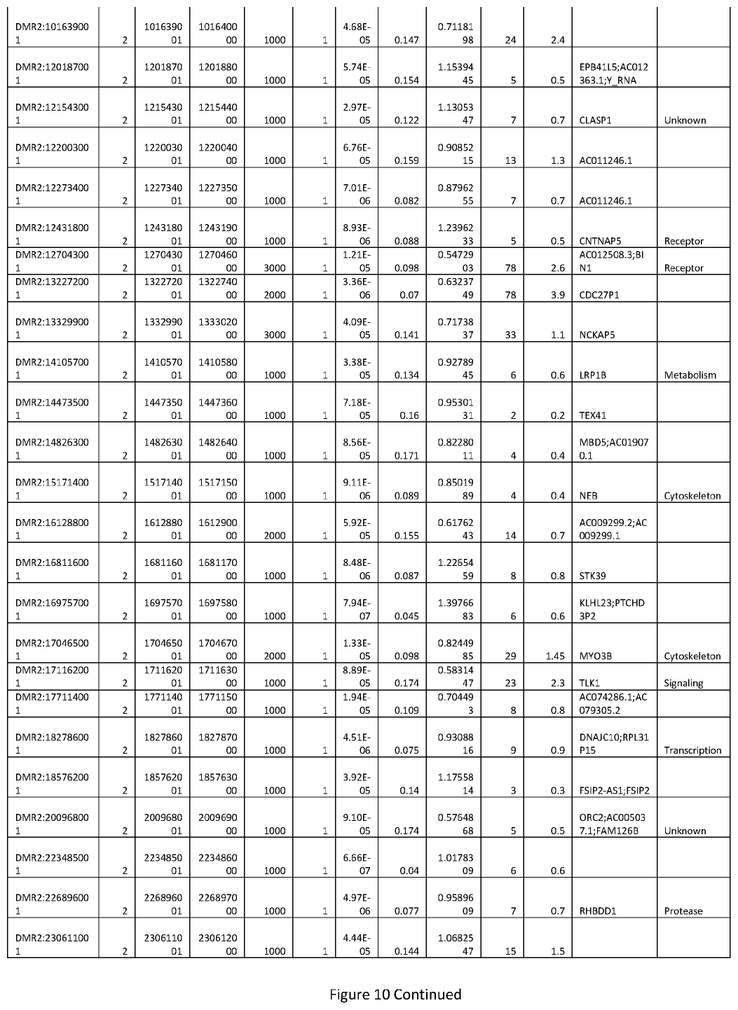

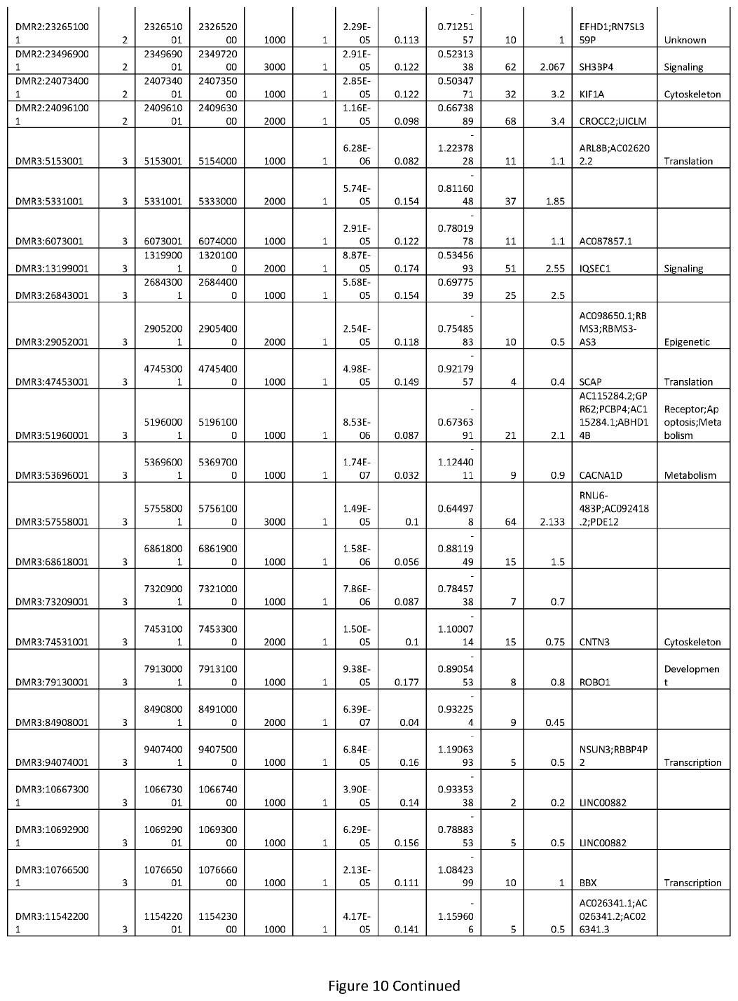

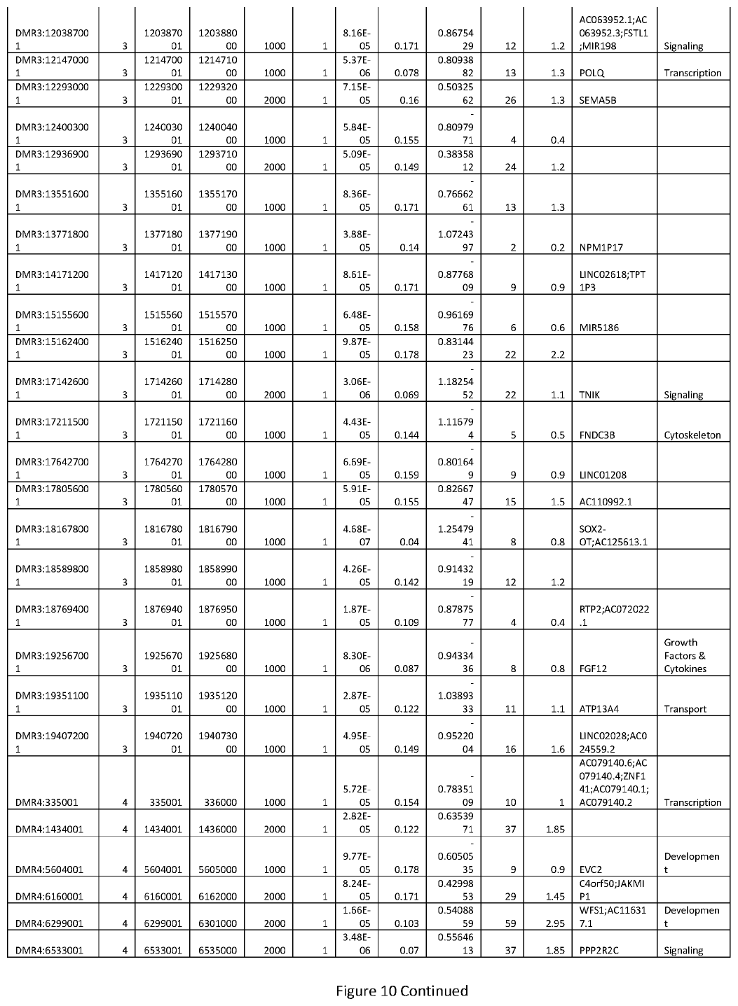

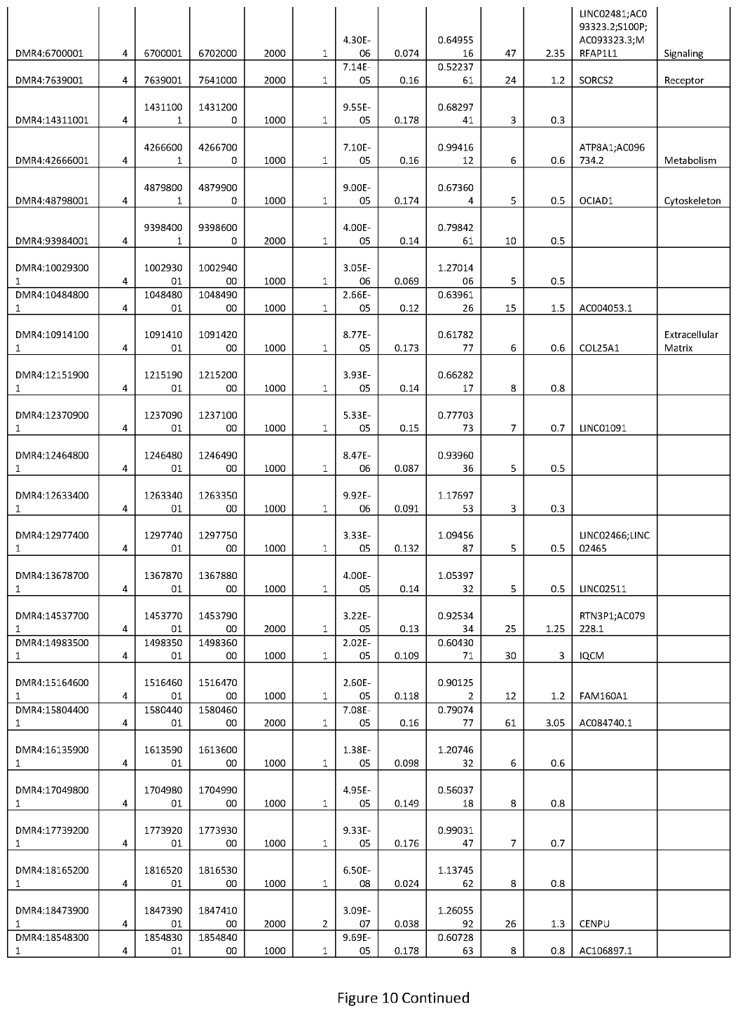

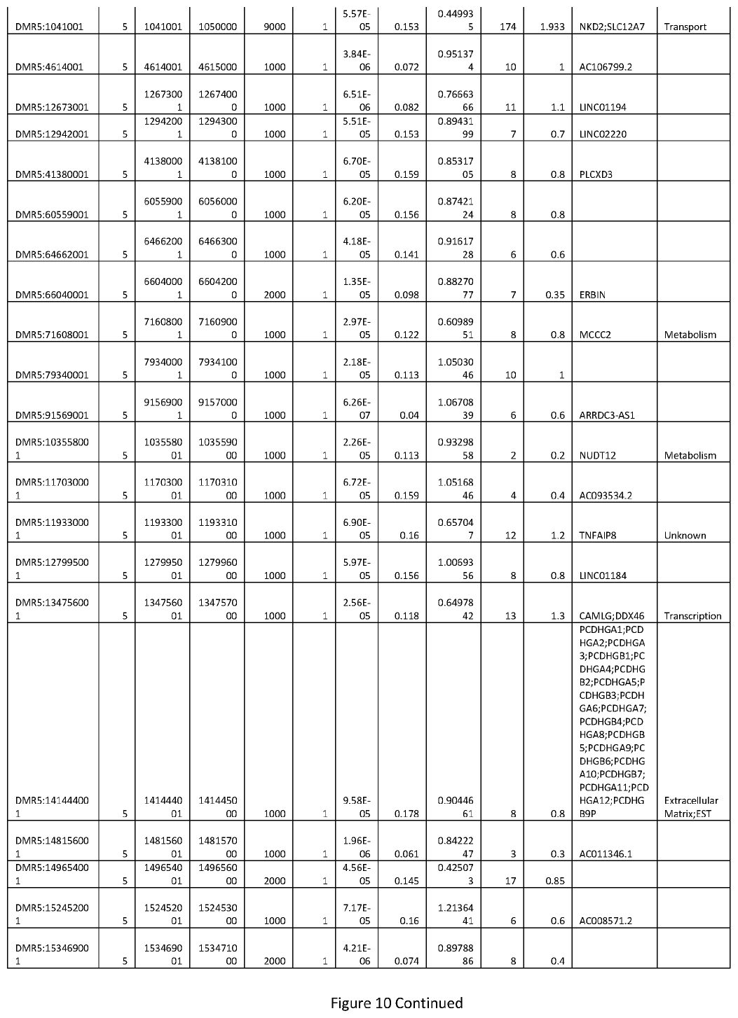

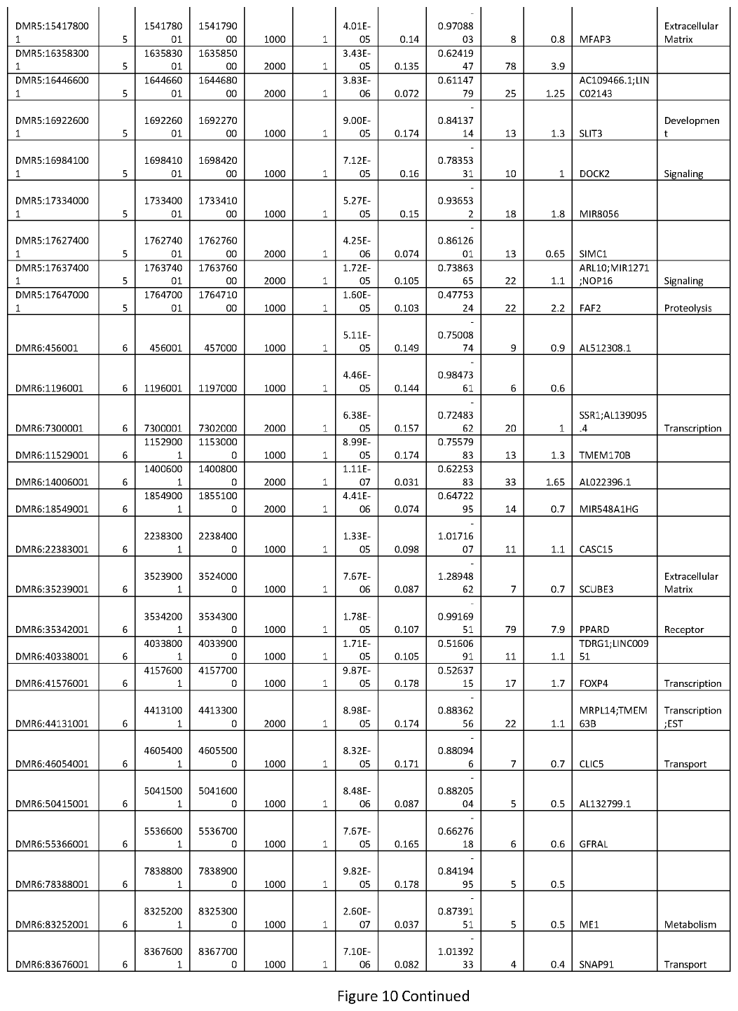

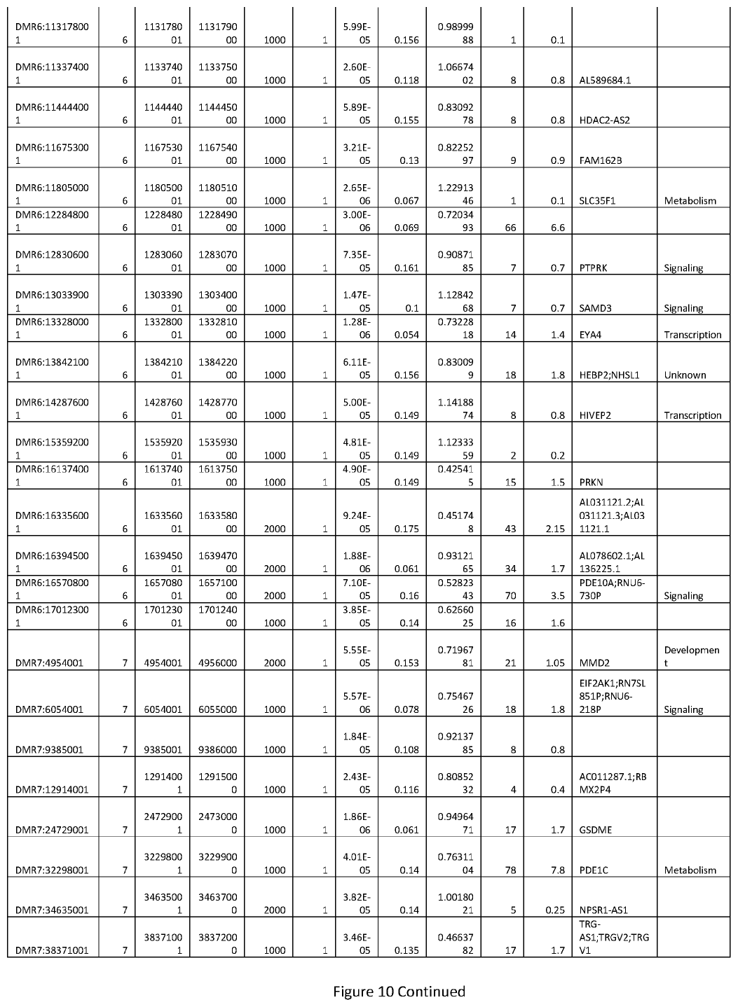

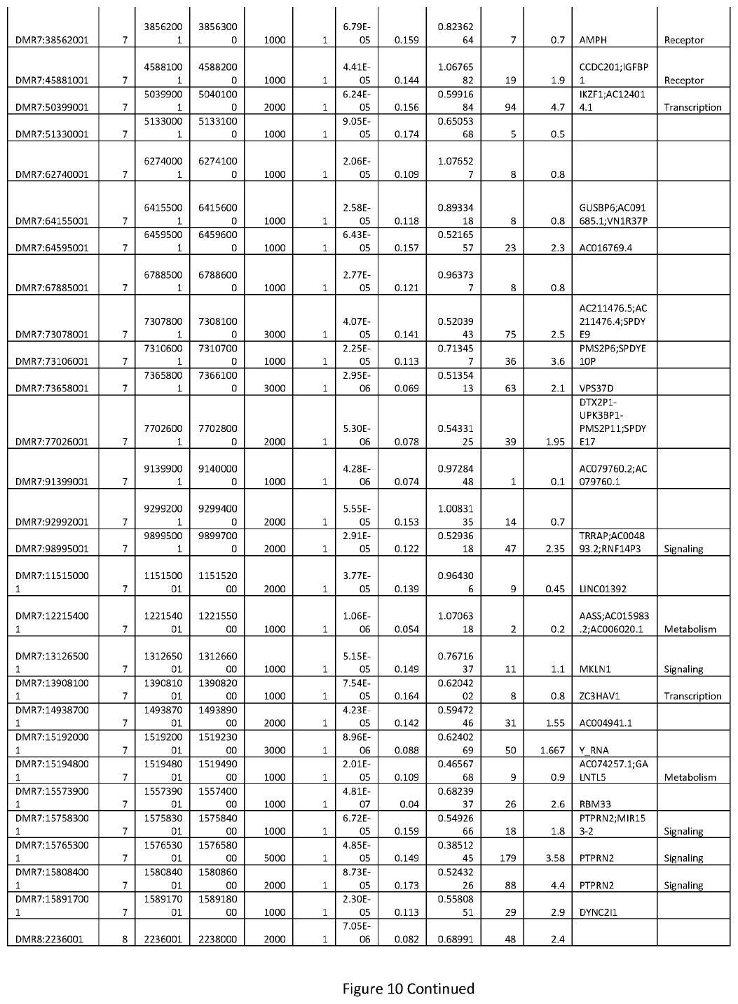

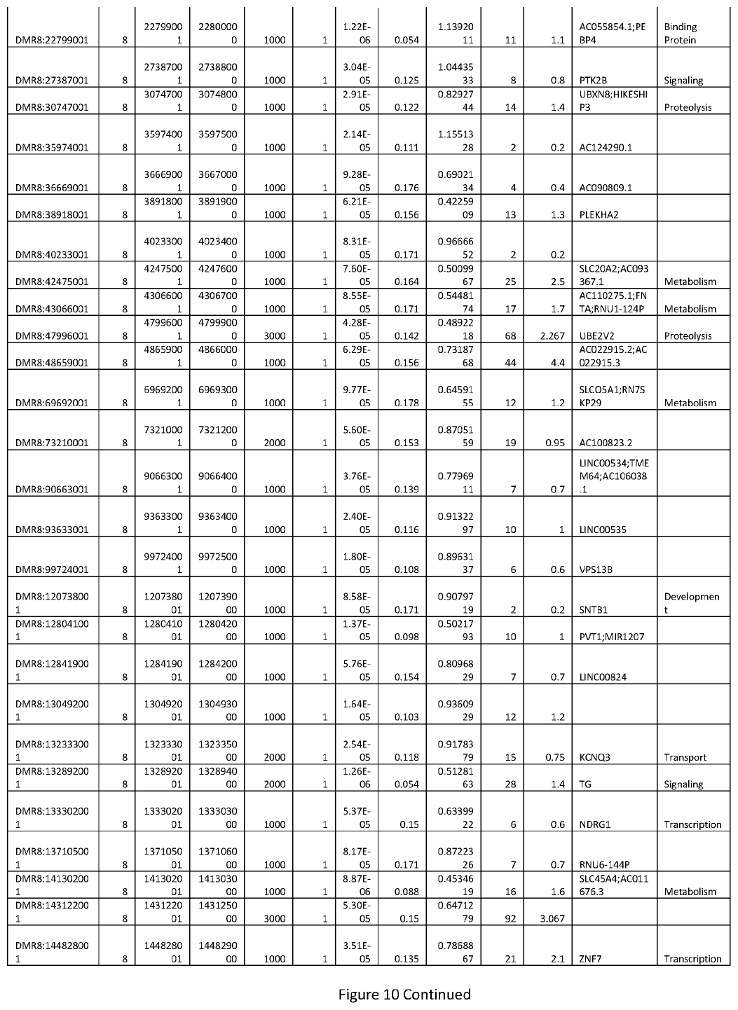

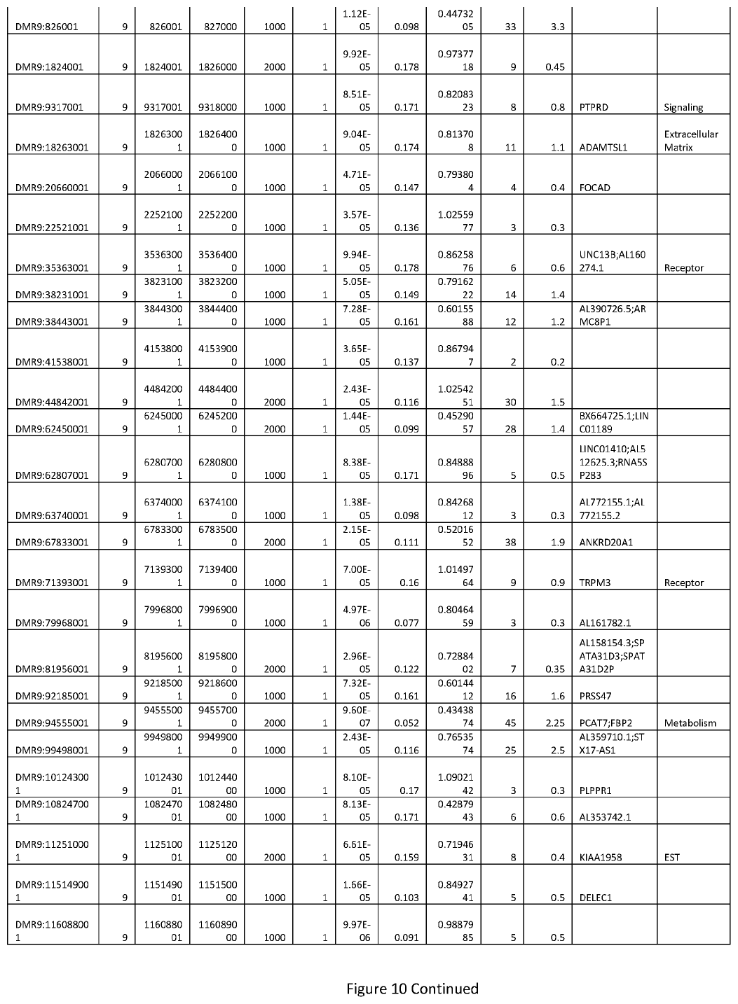

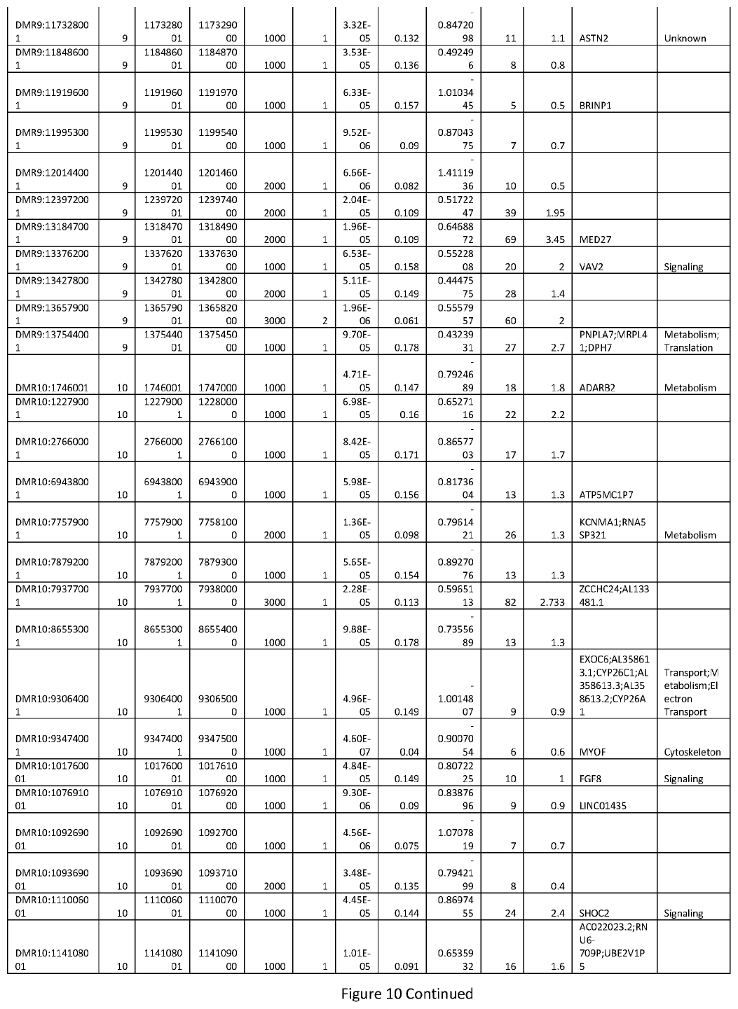

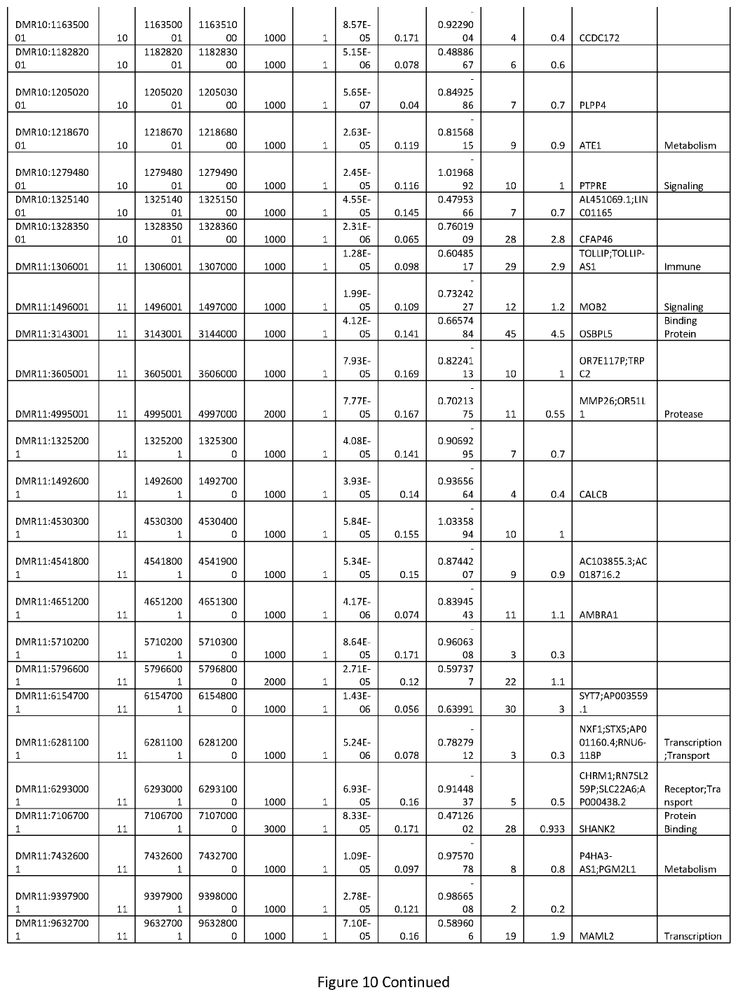

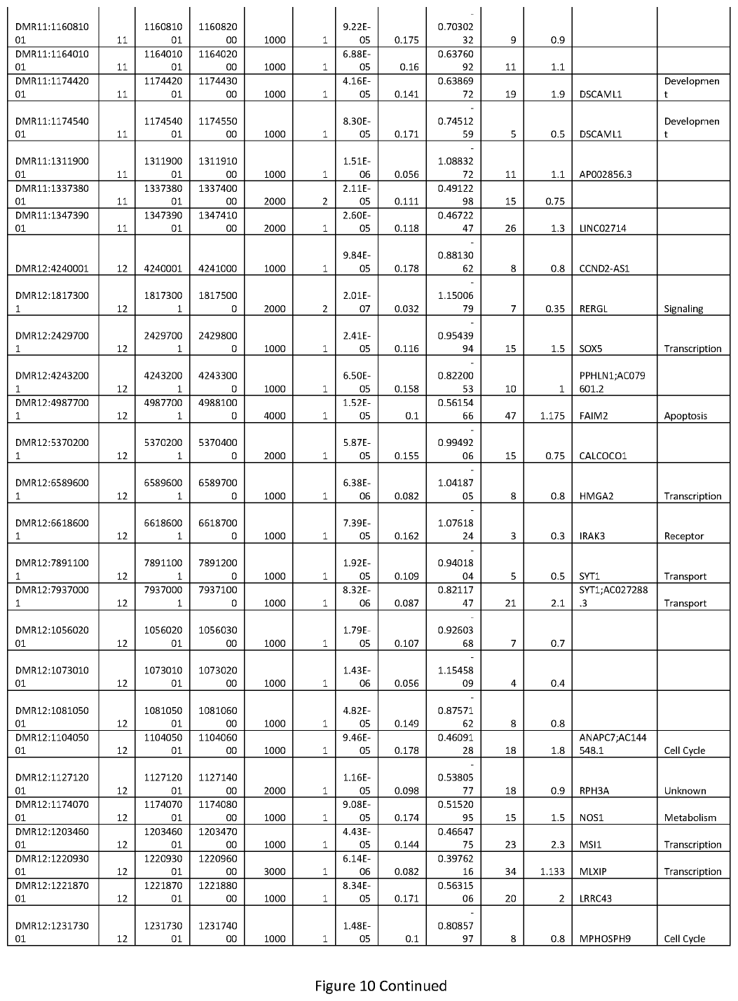

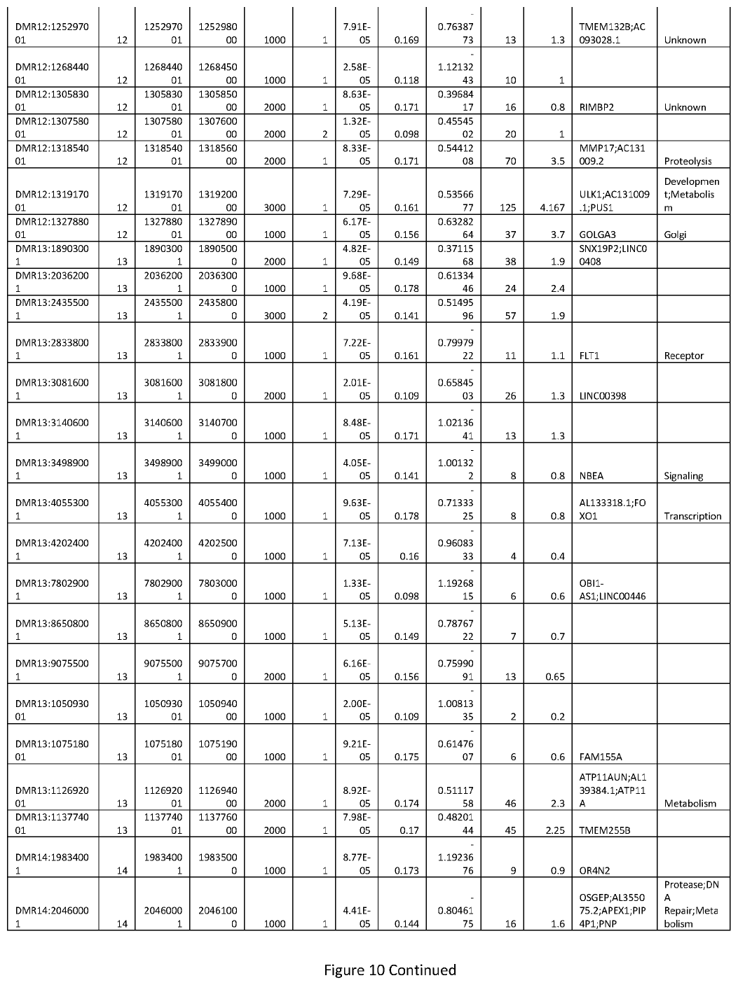

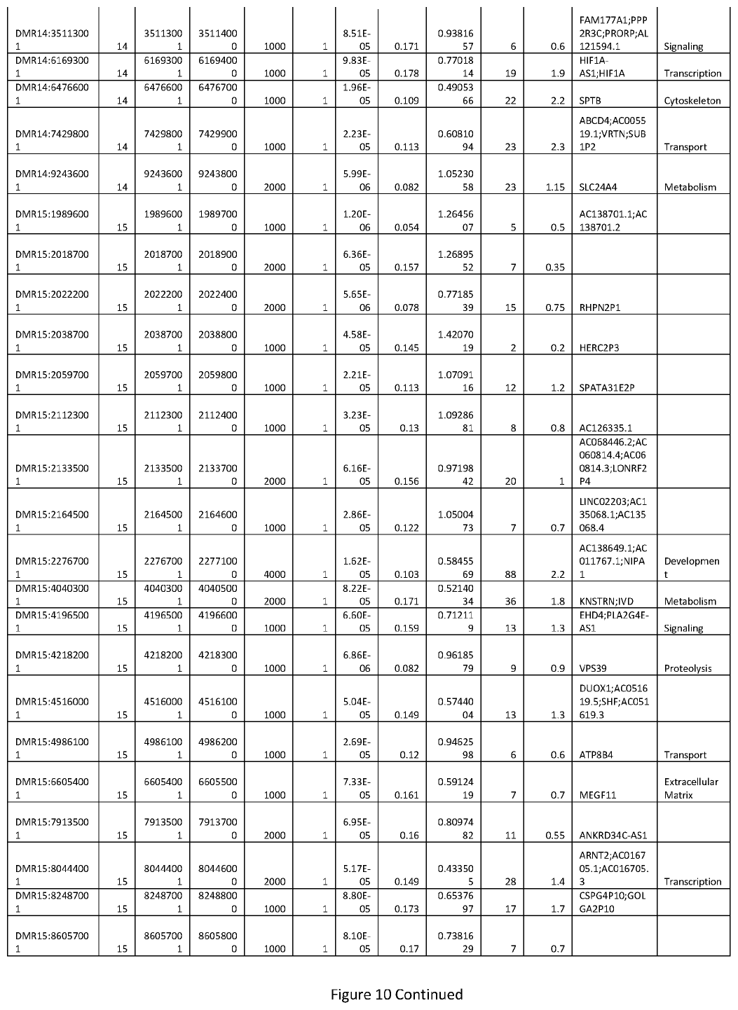

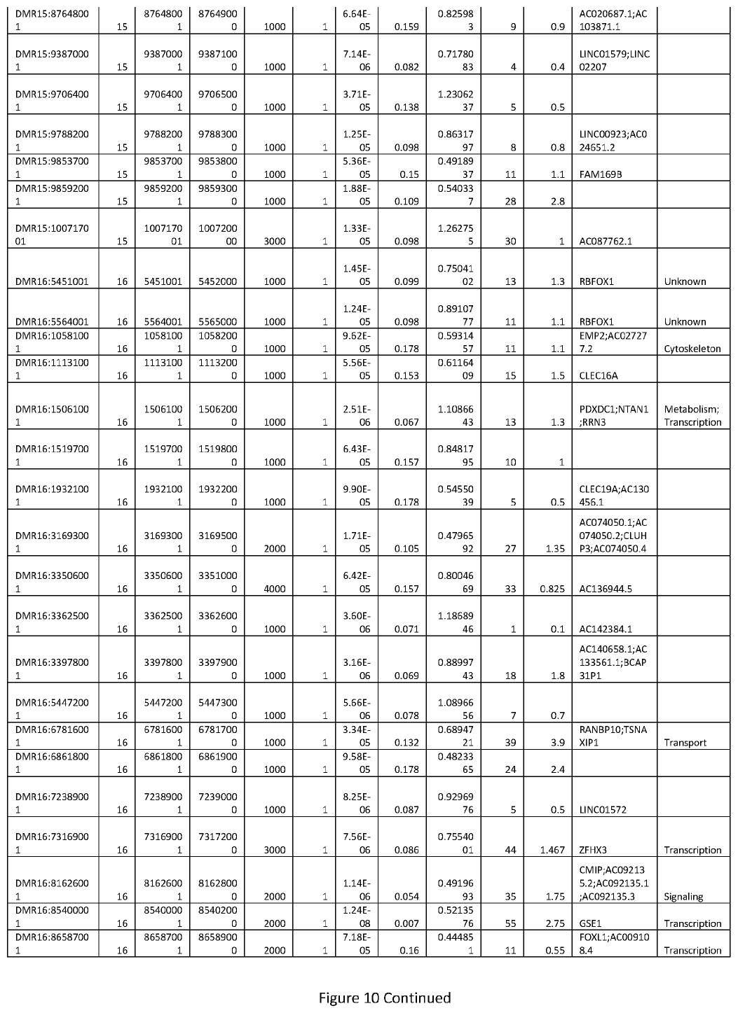

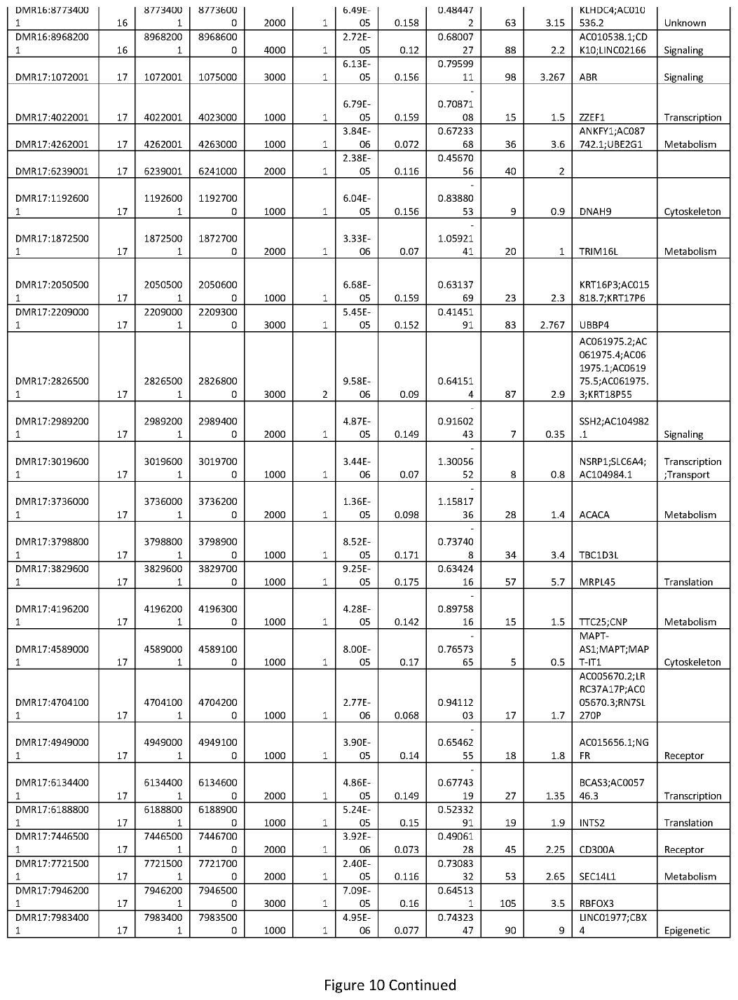

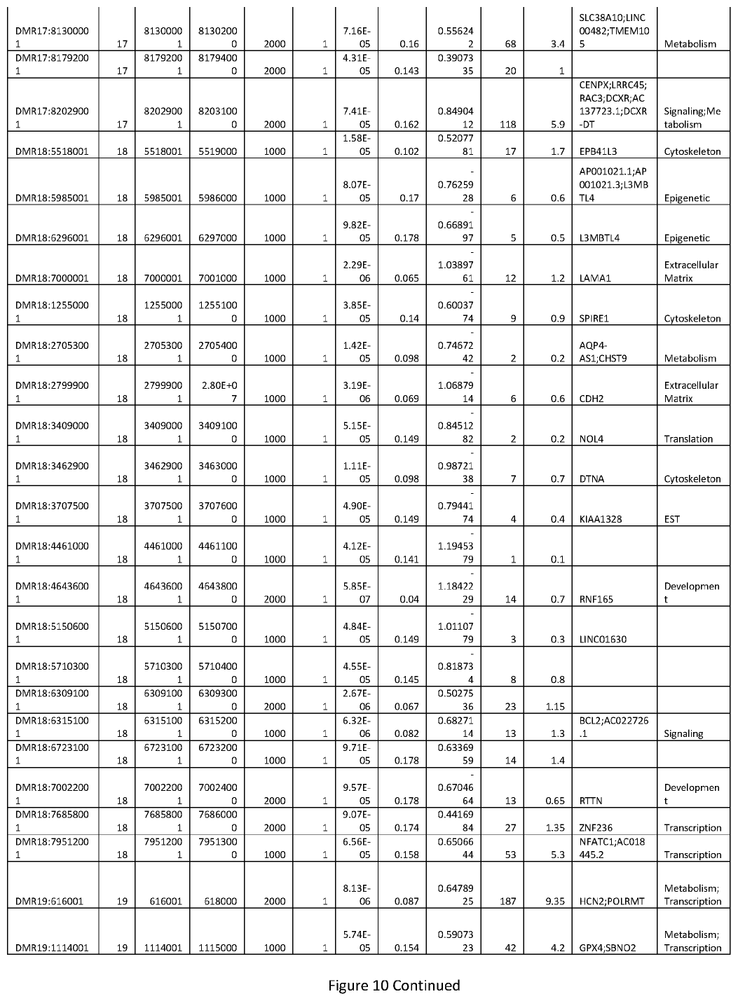

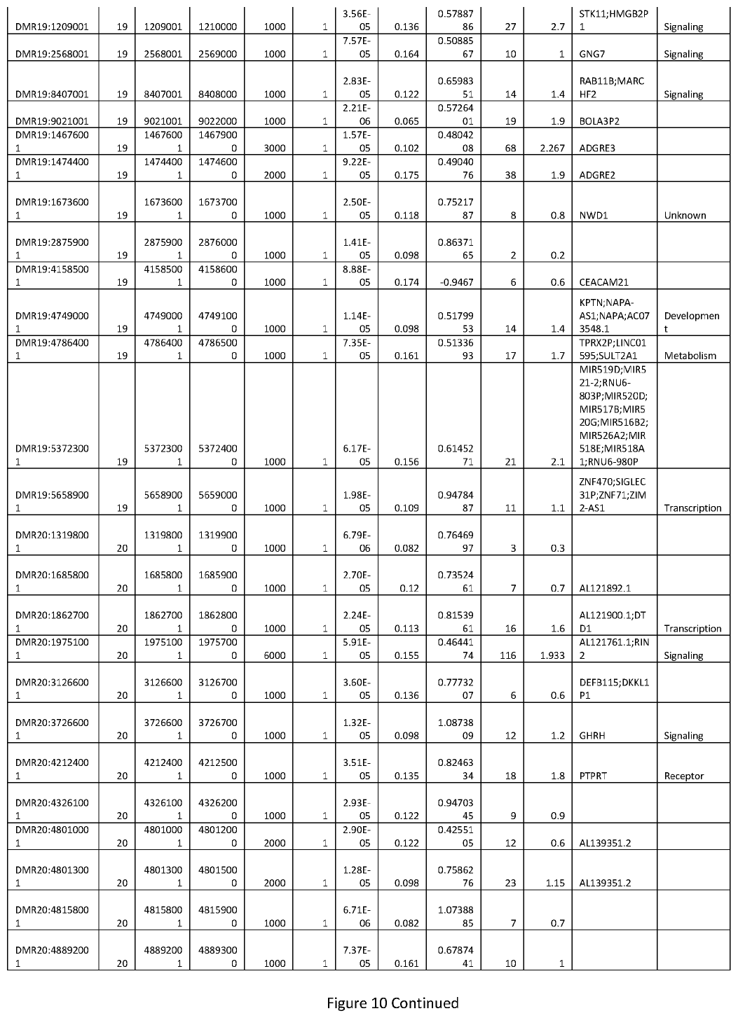

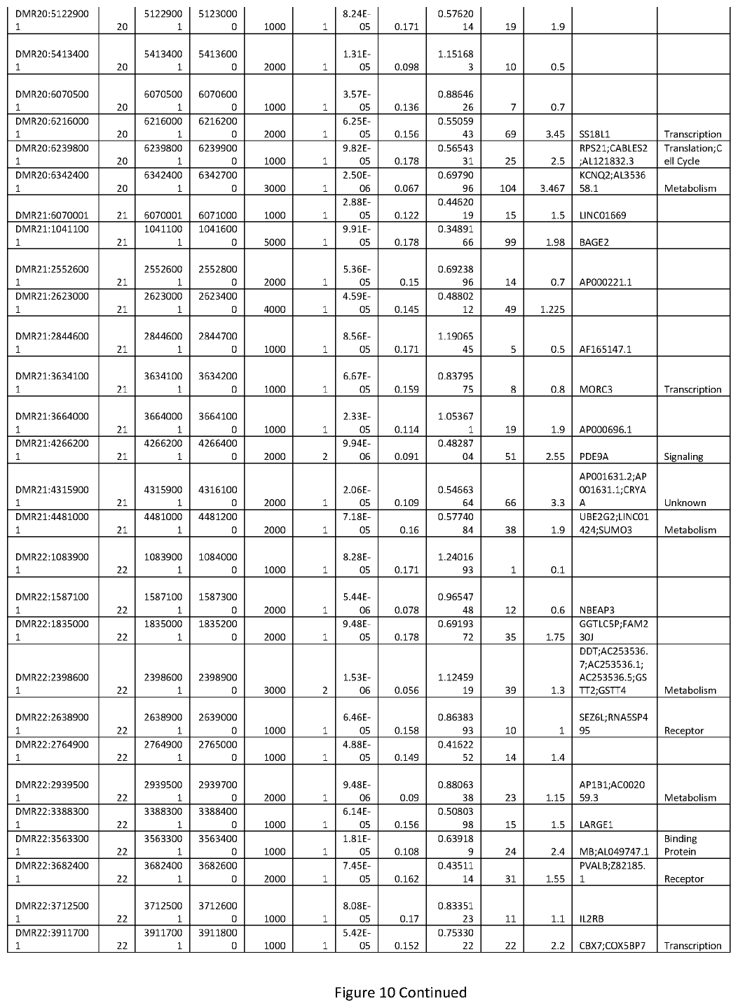

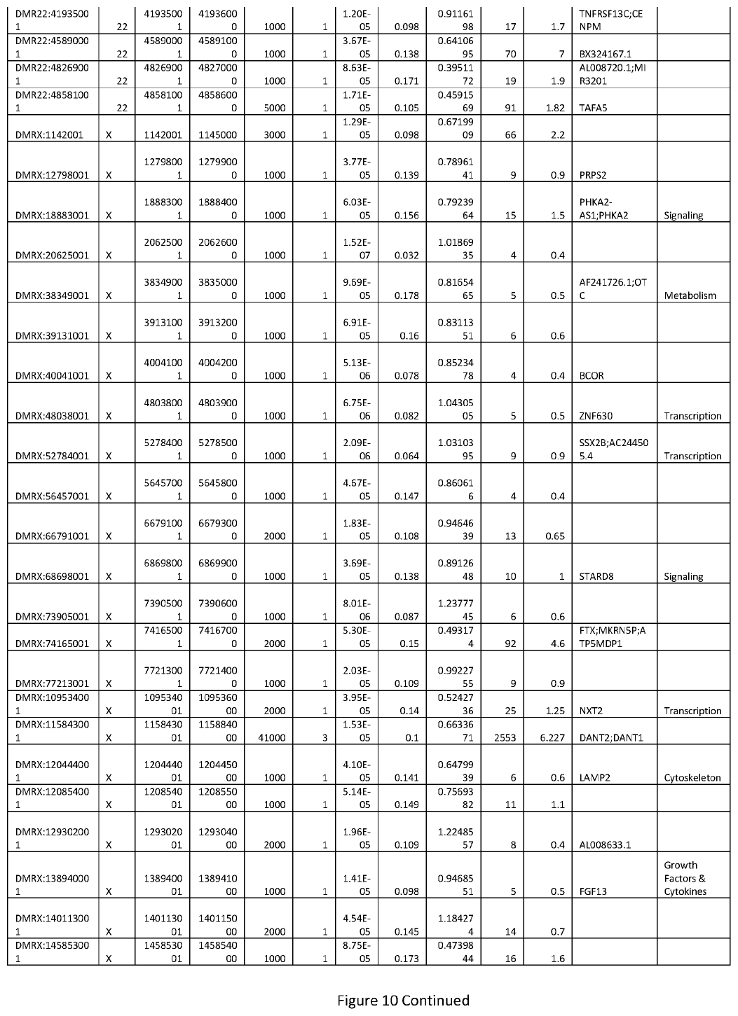

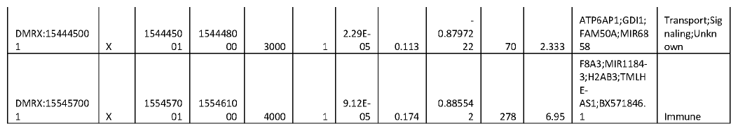

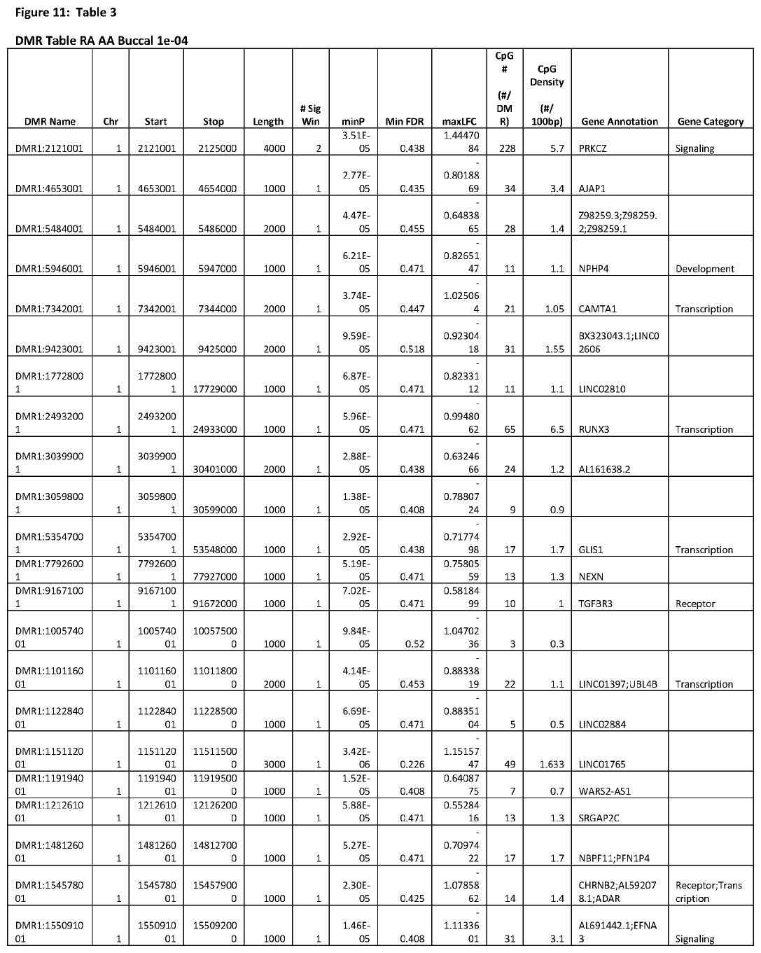

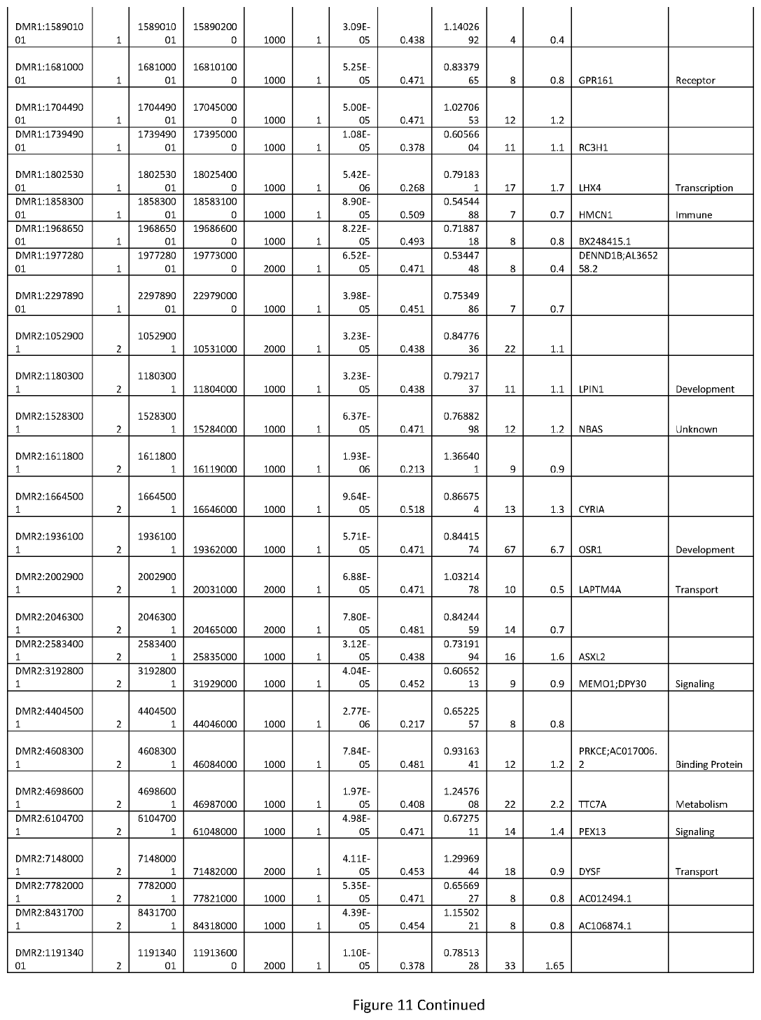

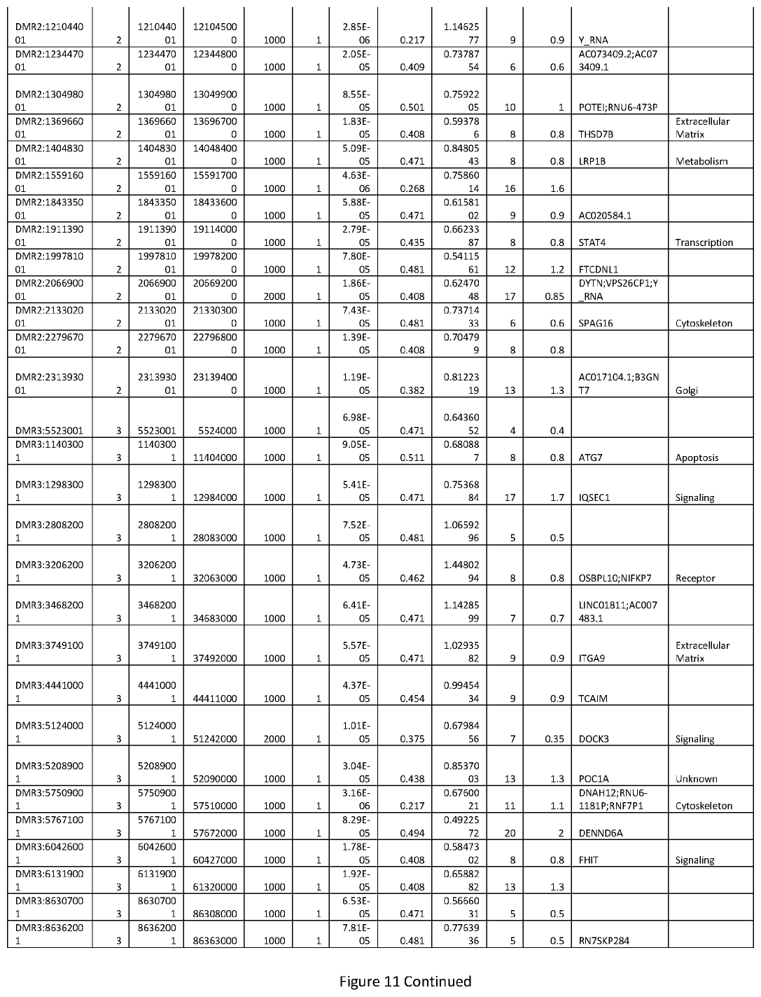

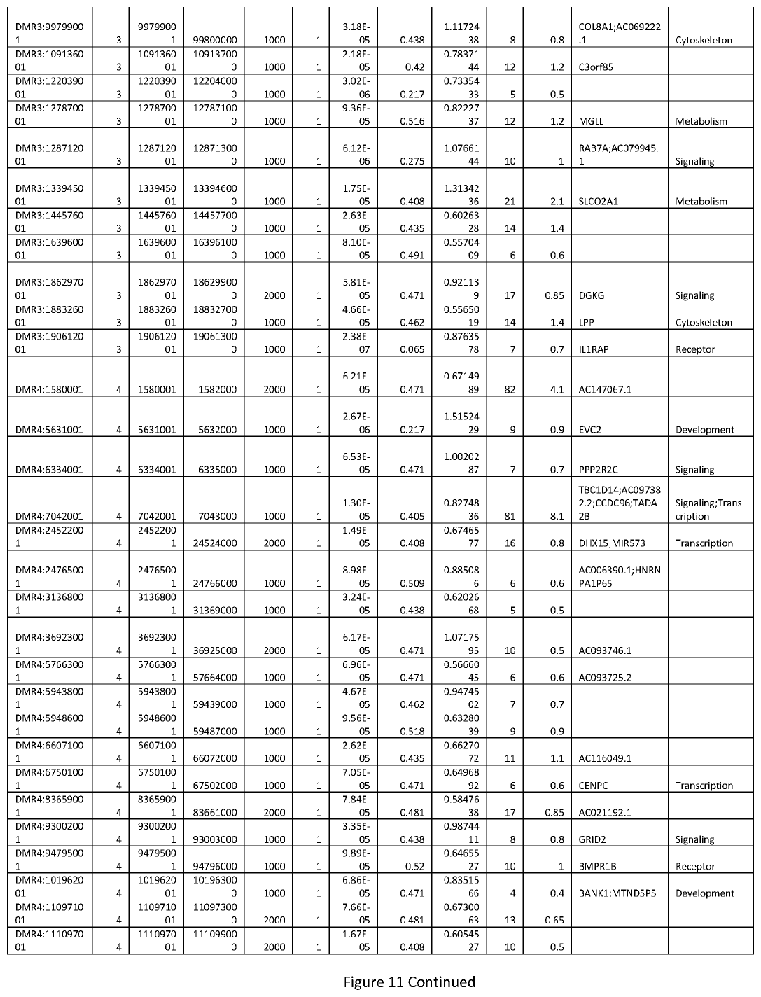

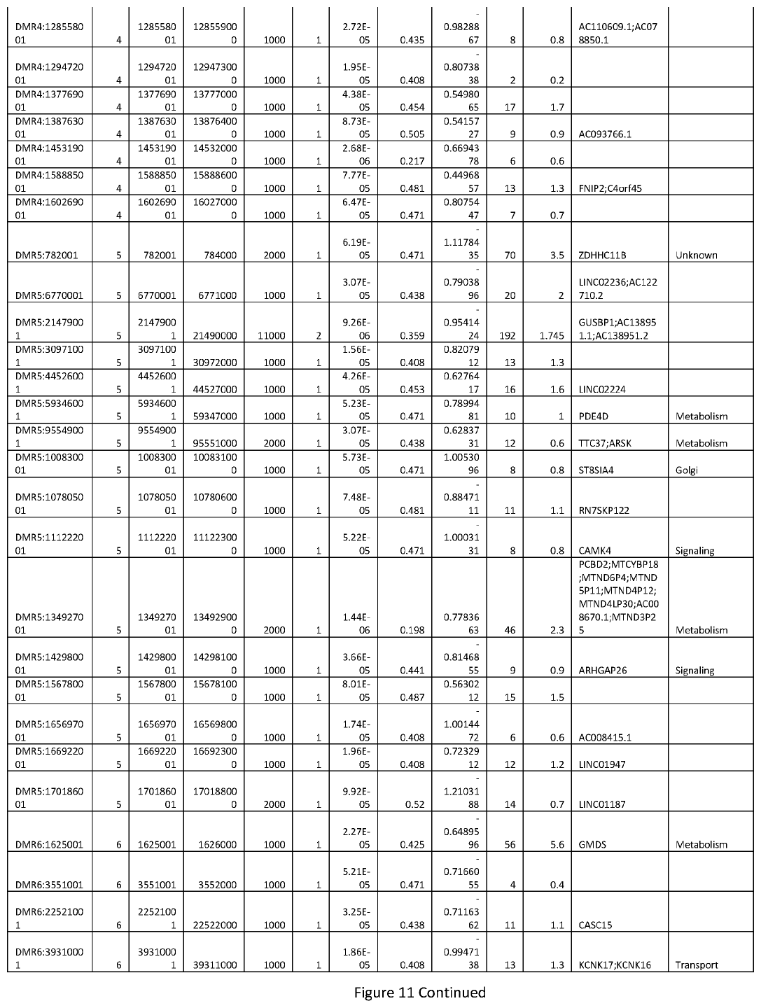

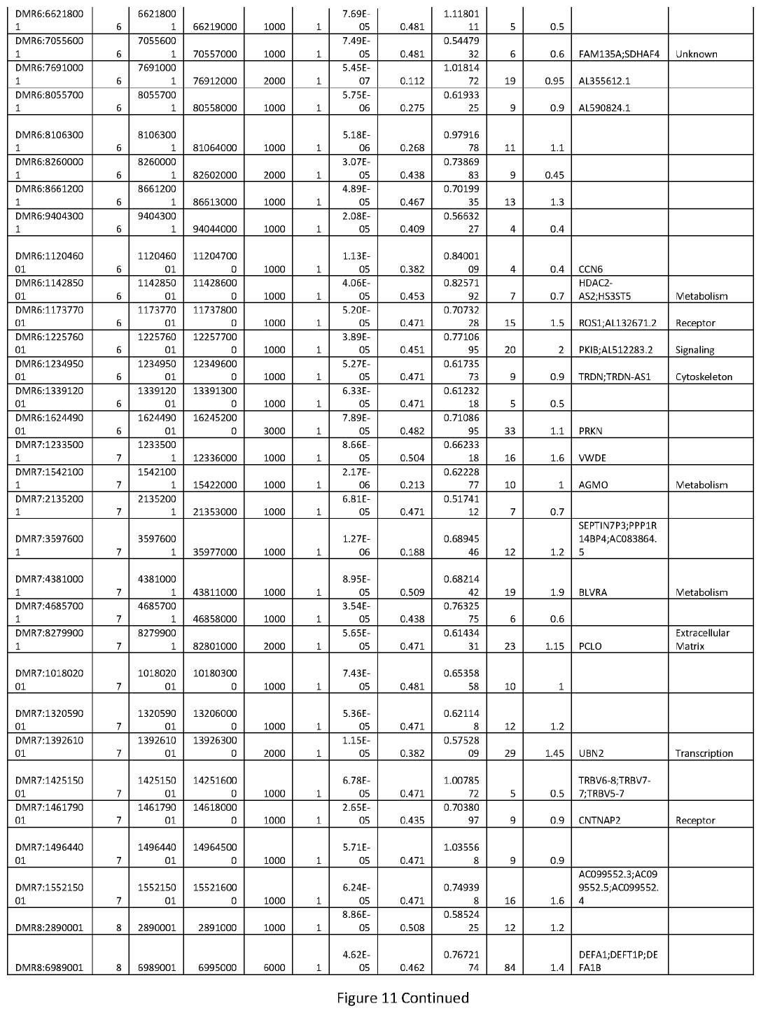

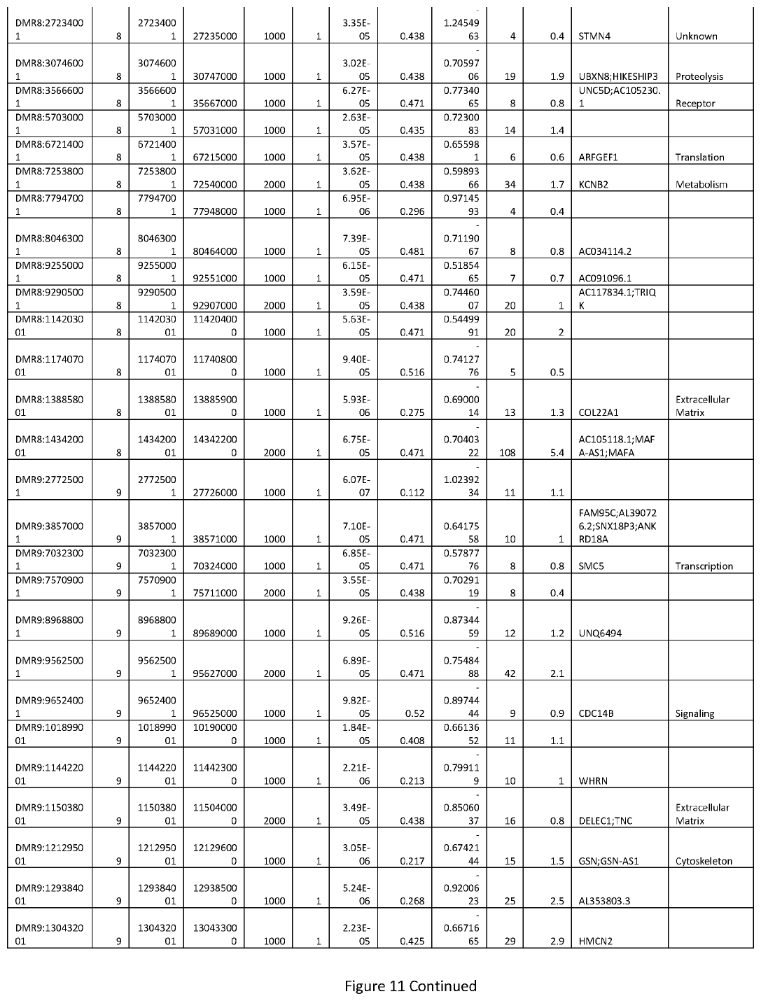

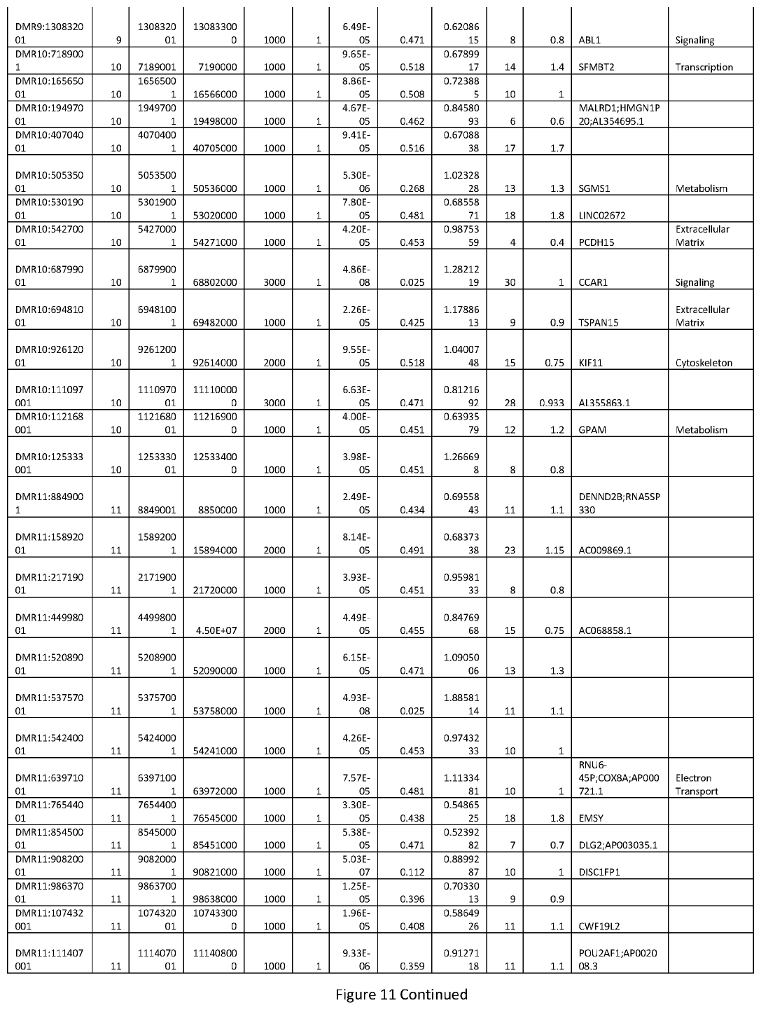

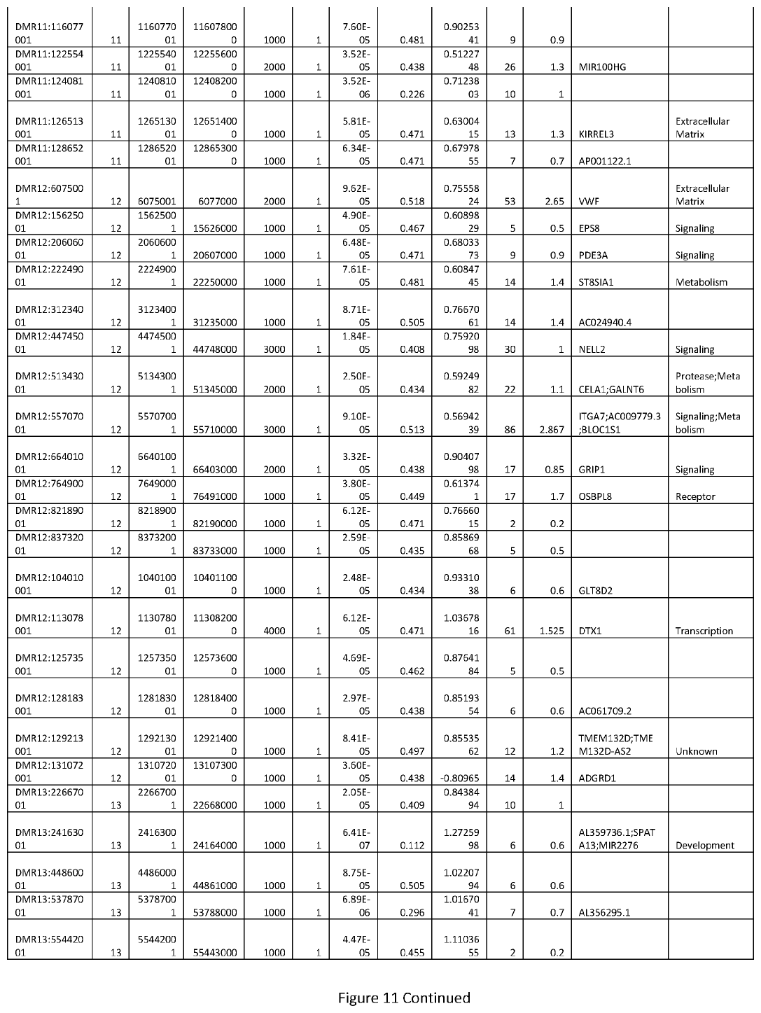

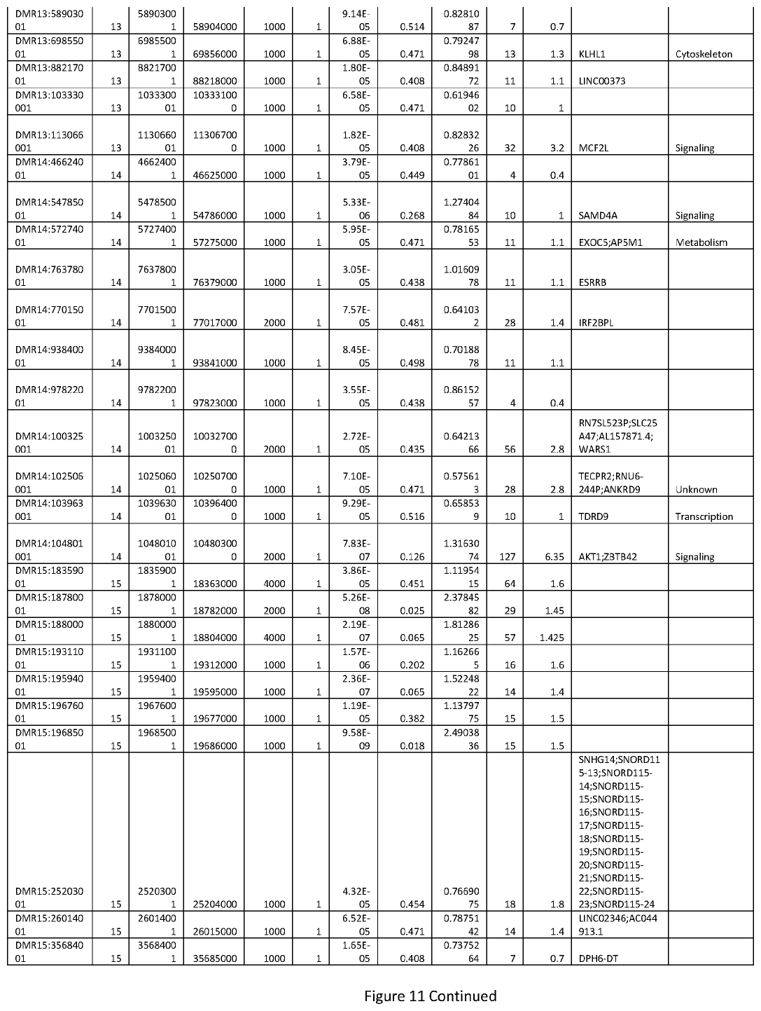

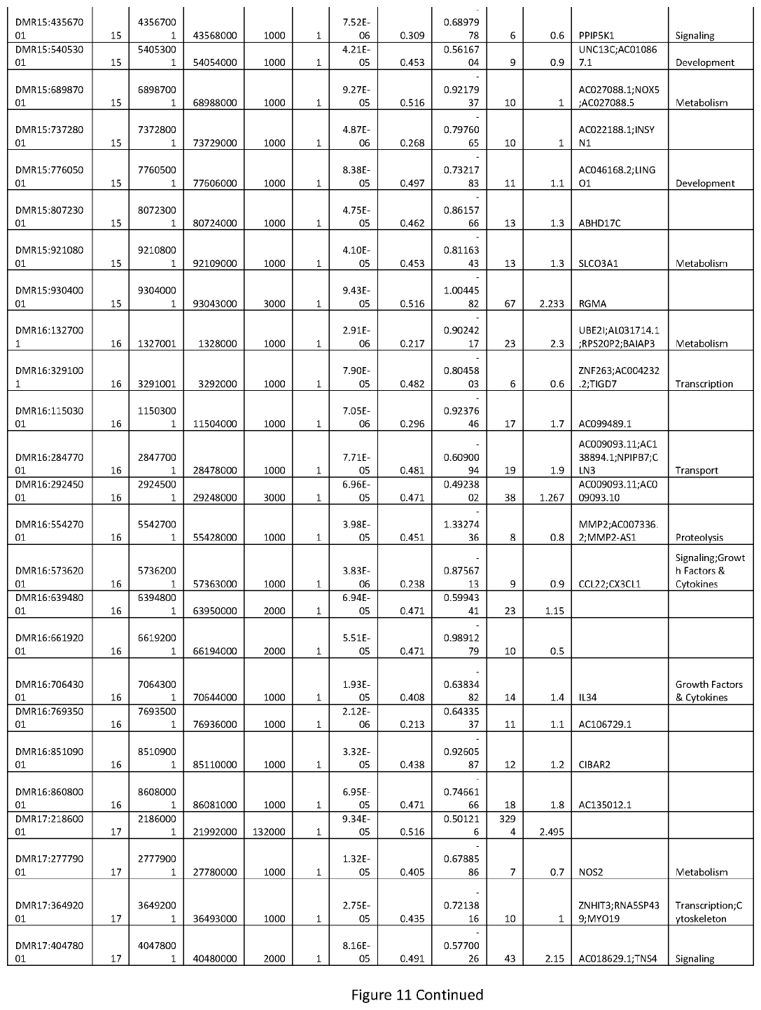

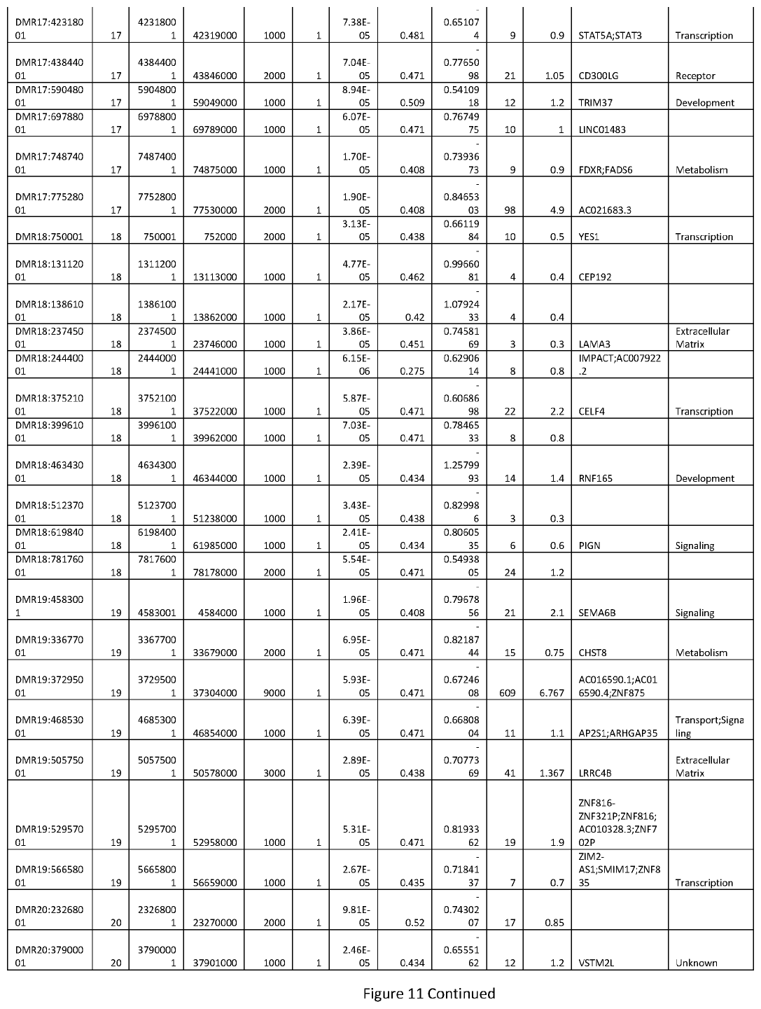

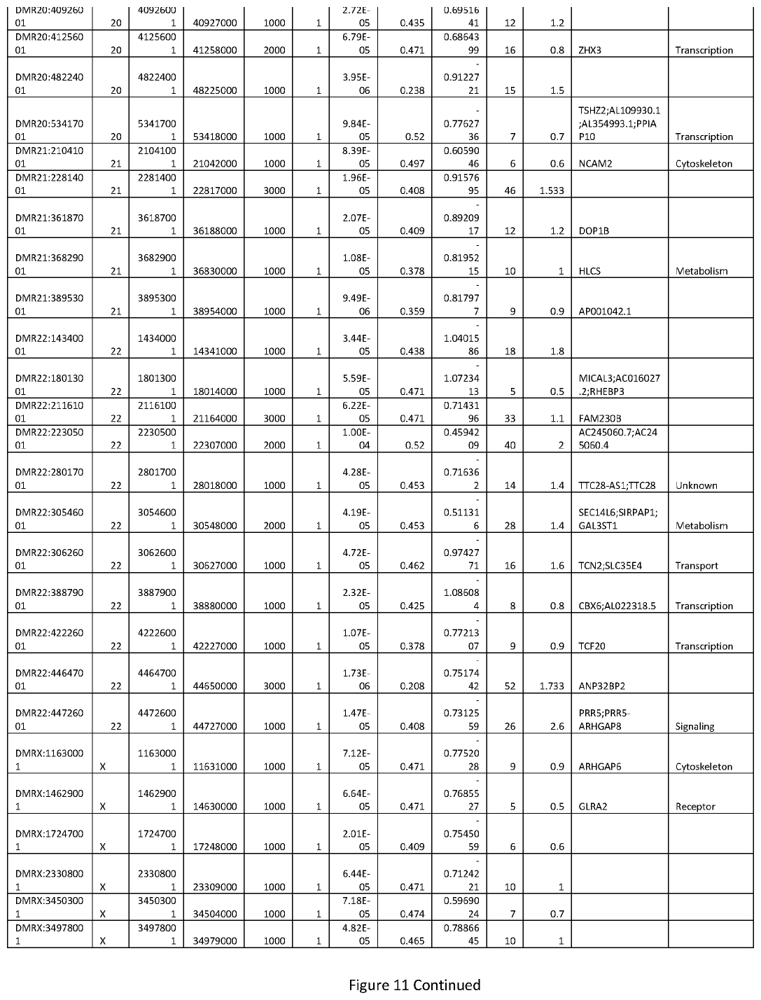

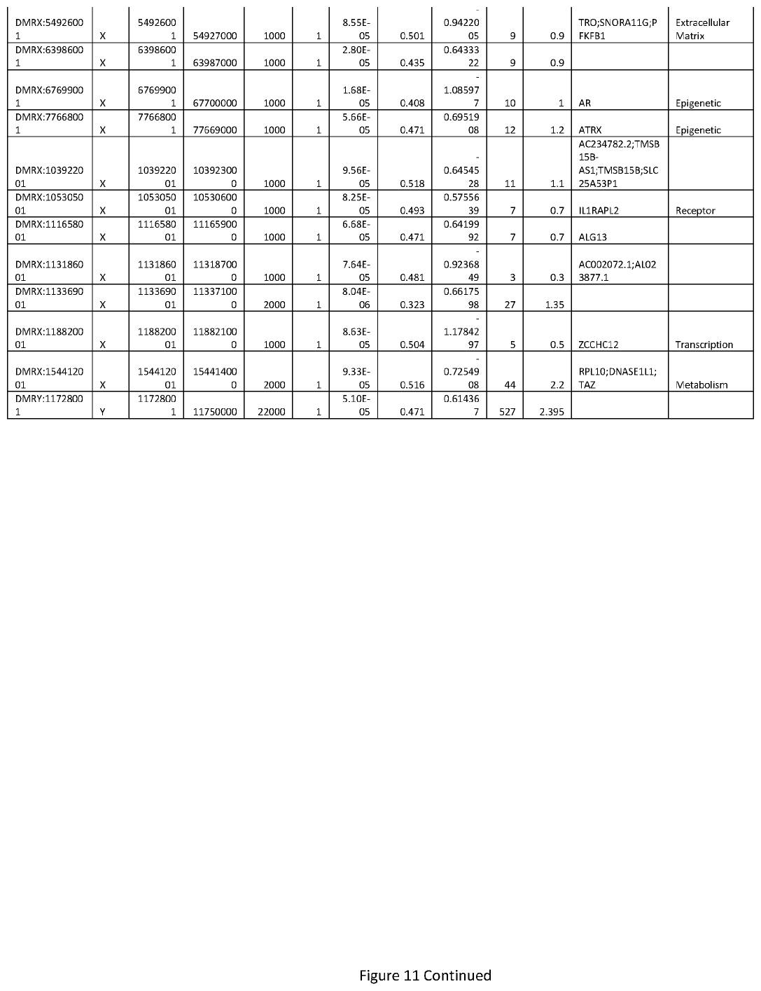

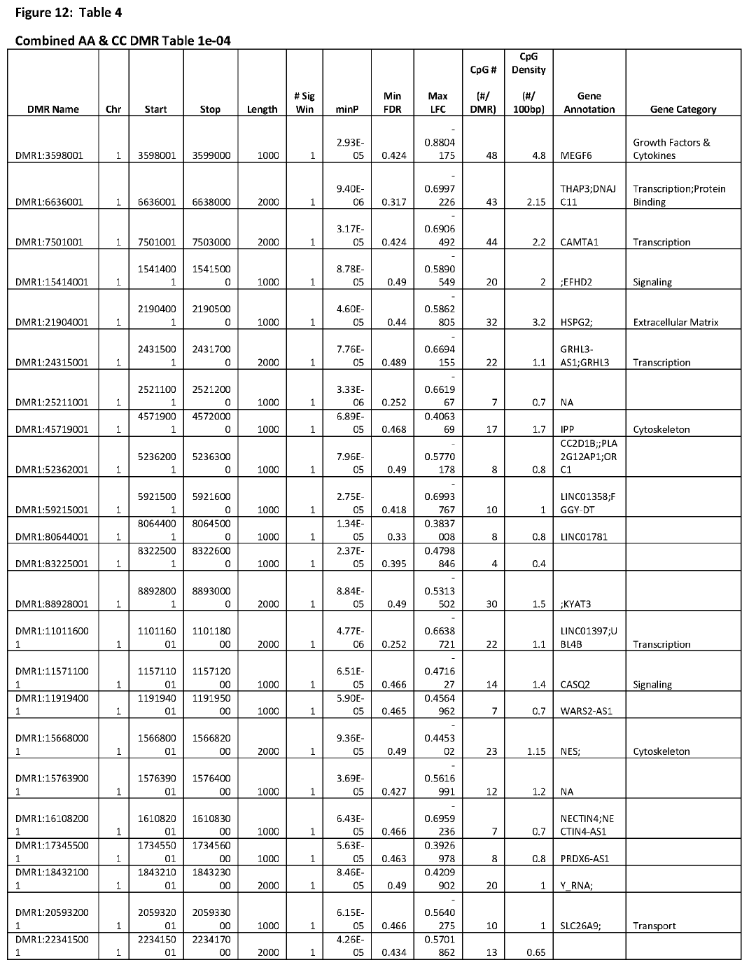

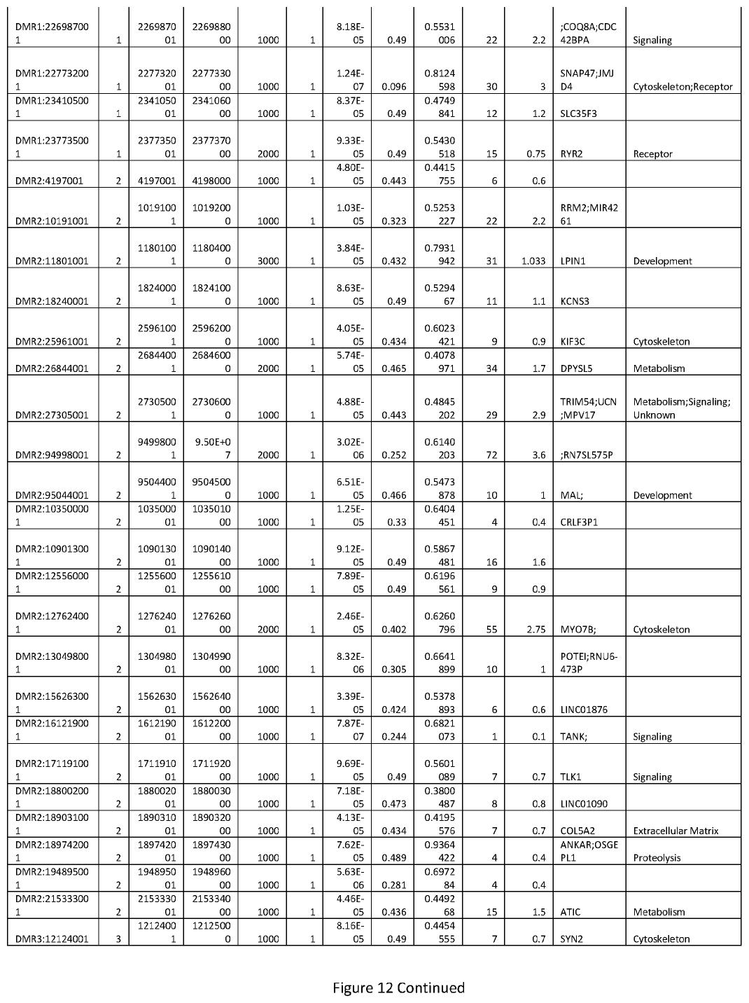

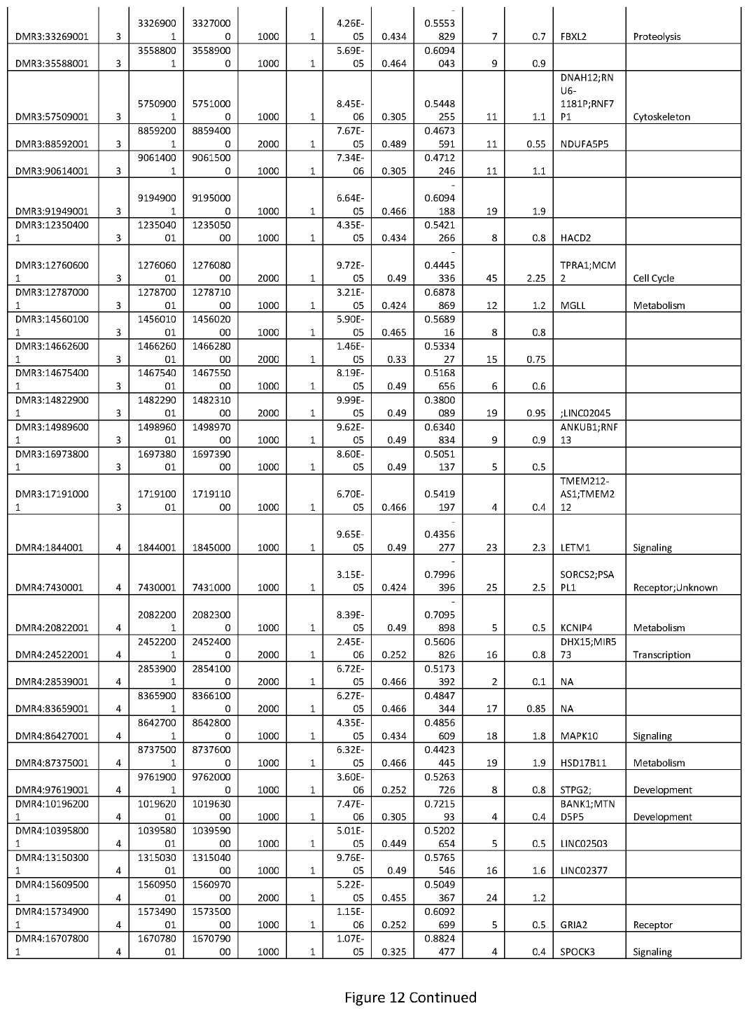

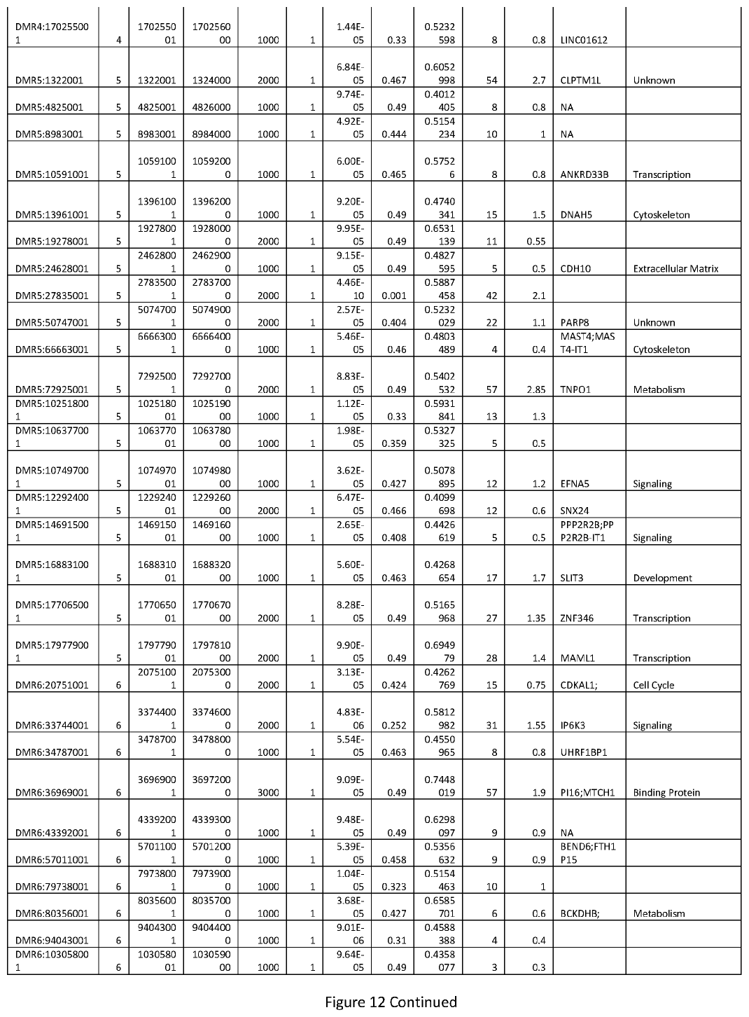

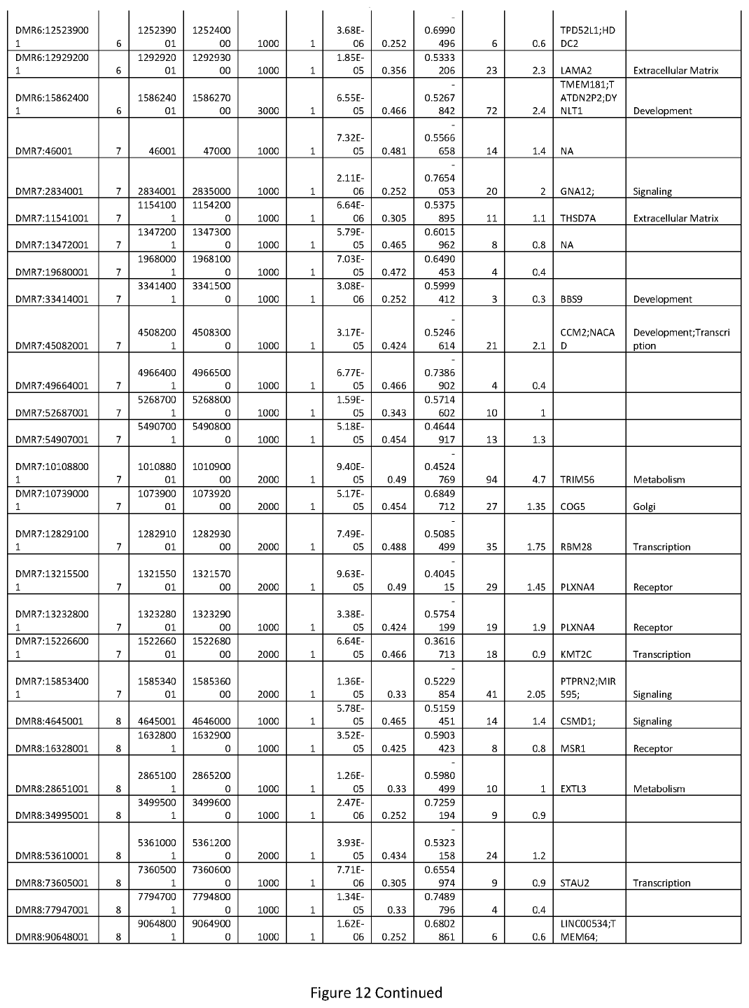

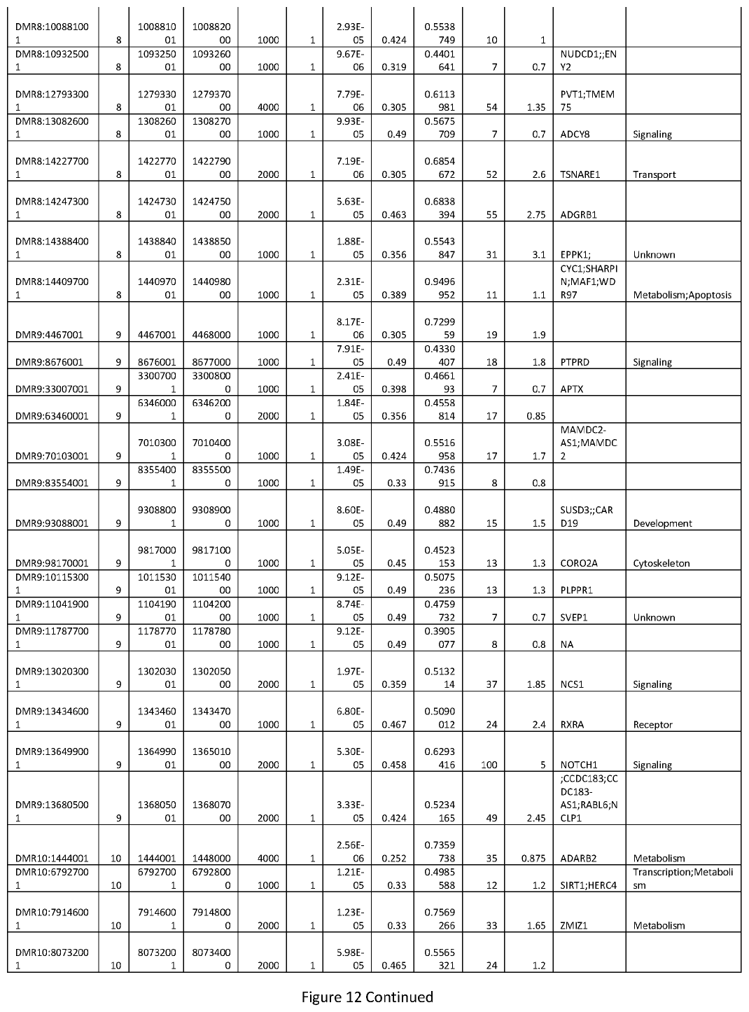

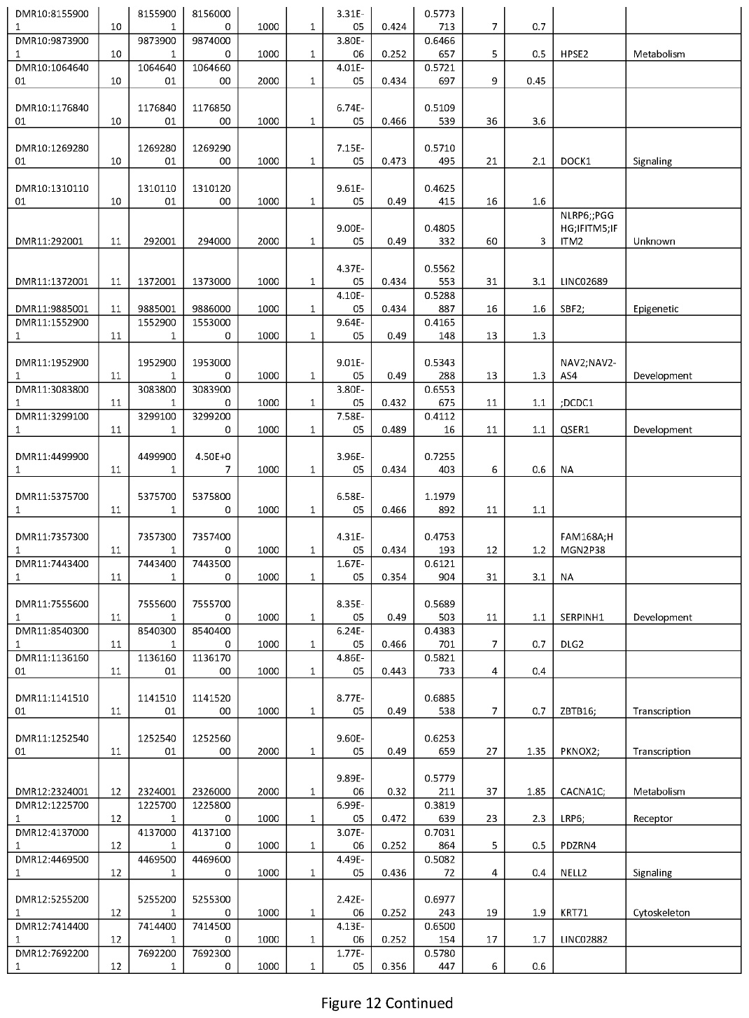

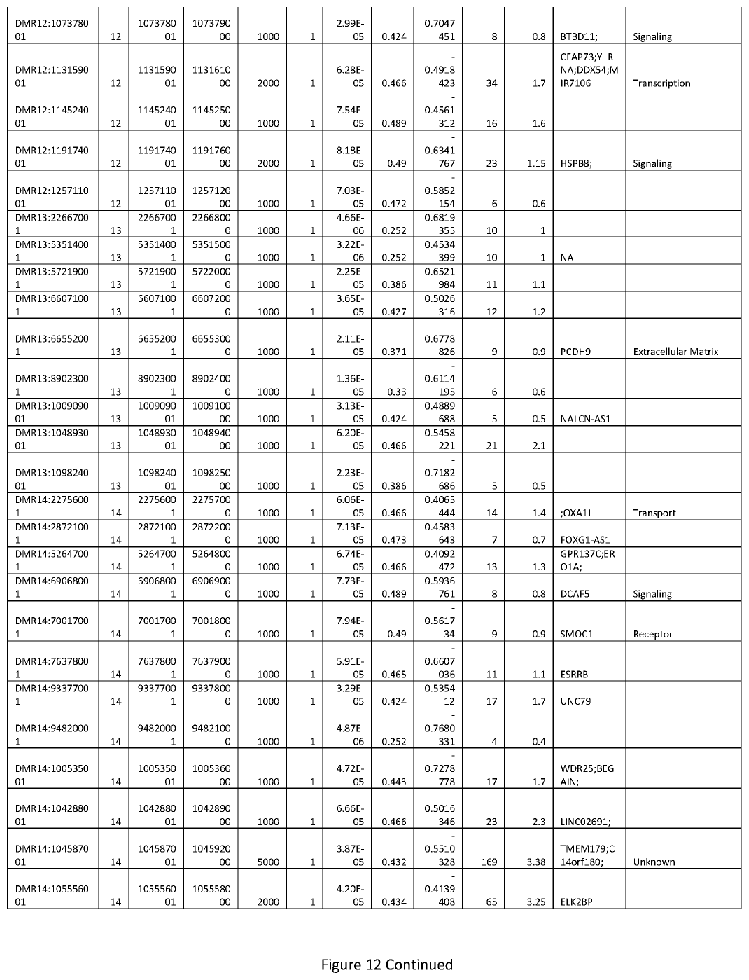

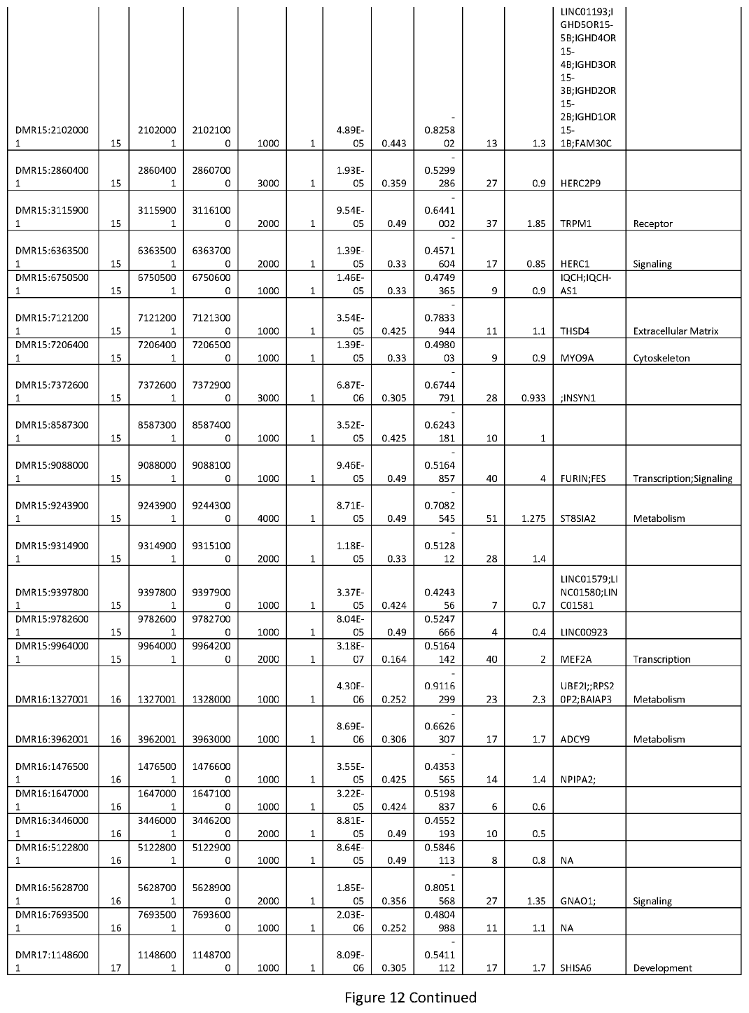

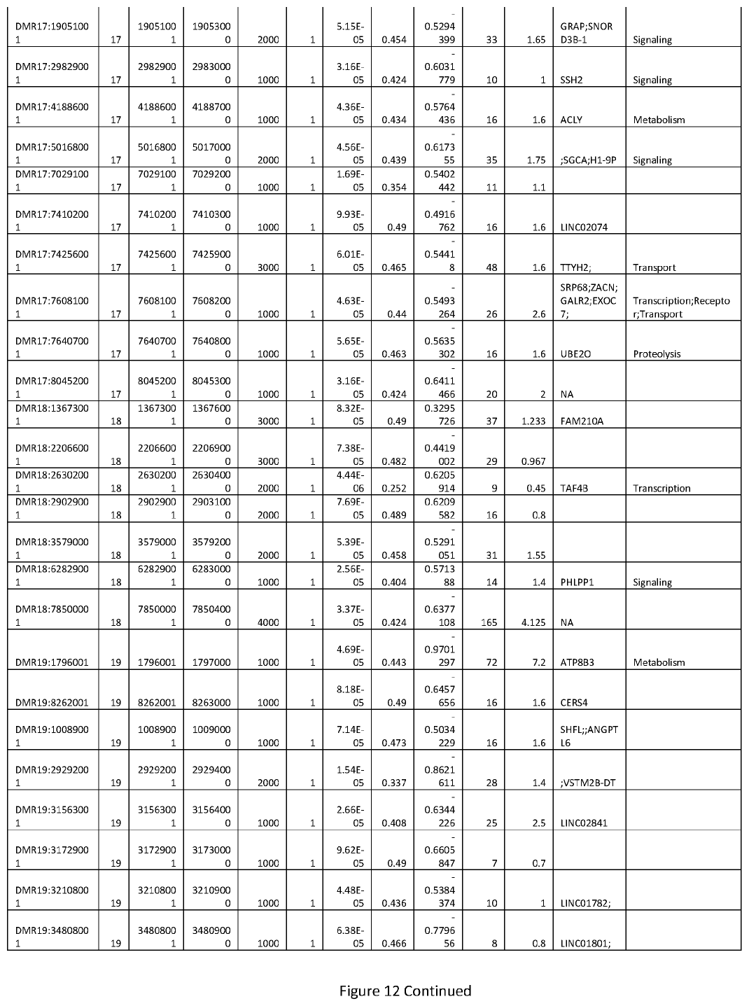

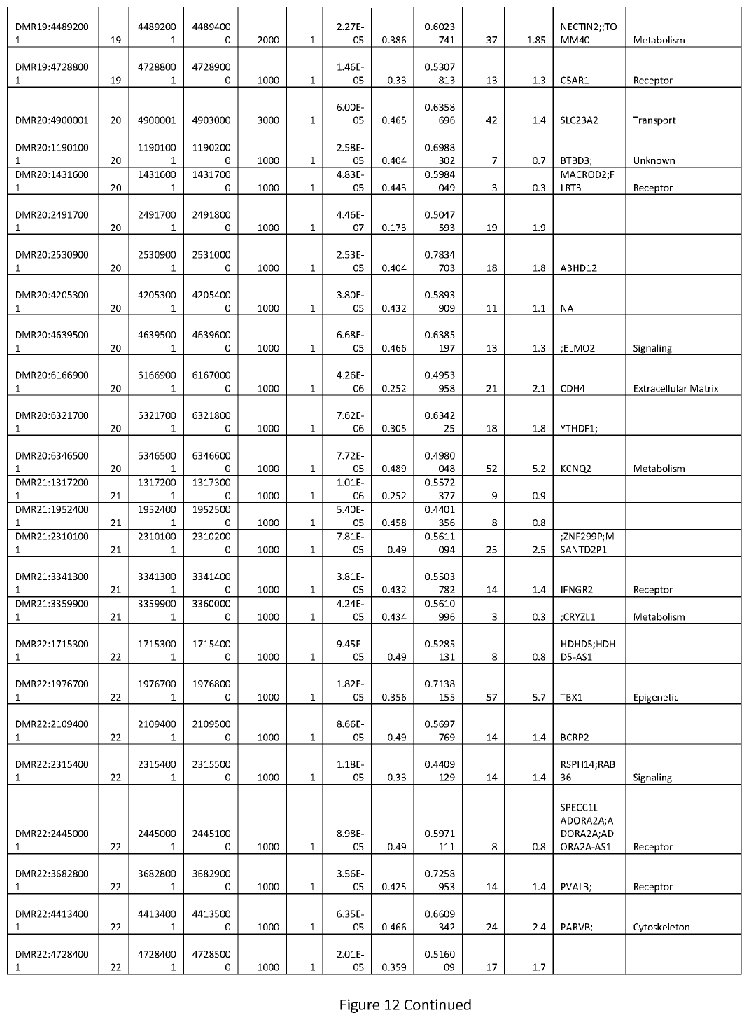

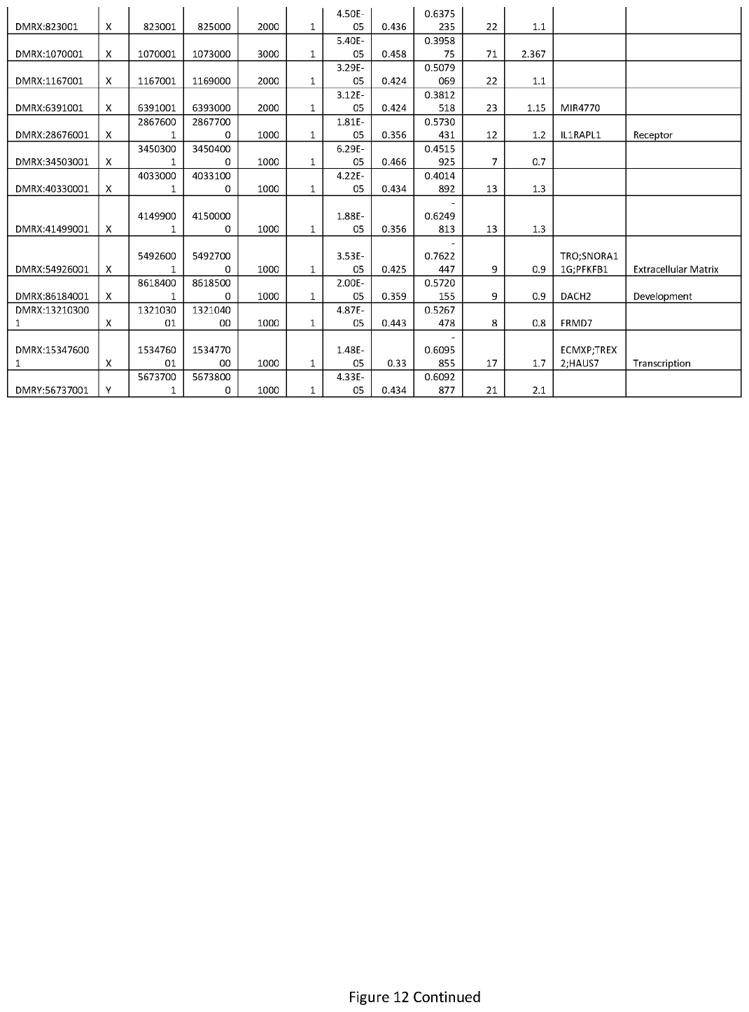

In an embodiment, the plurality of distinct DMRs are selected from the DMRs in Table 1, the DMRs in Table 2, the DMRs in Table 3, the DMRs in Table 4, and any combination thereof. In an embodiment, the plurality of distinct DMRs are selected from the DMRs in Table 1. FIG. 9 displays Table 1. In an embodiment, the plurality of distinct DMRs are selected from the DMRs in Table 2. FIG. 10 displays Table 2. In an embodiment, the plurality of distinct DMRs are selected from the DMRs in Table 3. FIG. 11 displays Table 3. In an embodiment, the plurality of distinct DMRs are selected from the DMRs in Table 4. FIG. 12 displays Table 4.

In some embodiments, a method can comprise: obtaining a sample of somatic cells from a human subject; isolating deoxyribonucleic acid (DNA) from the sample; detecting and determining a methylation level of a differential DNA methylation region (DMR) comprised in the isolated DNA; and comparing the methylation level of the DMR to a reference methylation level of a corresponding reference DMR. In some cases, a reference DMR can be from a reference (e.g., control) sample. In some cases, a reference sample can be from a healthy population (e.g., a population that does not have rheumatoid arthritis) or a diseased population (e.g., a population with rheumatoid arthritis). In some cases, the comparing comprises comparing employing a computer comprising a computer processor and computer readable memory comprising computer readable instructions. In some cases, the detecting and determining can comprise a methylated DNA immunoprecipitation (MeDIP), a sequencing, a bisulfite treatment, a bisulfite conversion, a deamination of an unmethylated cytosine base, employing an array, or any combination thereof. In some cases, the methylation level of one or more distinct DMRs (for example from the DMRs disclosed in Table 1, Table 2, Table 3, Table 4 or any combination thereof) is detected and compared. In some embodiments, a method can comprise determining a methylation level in at least one distinct DMR. In some embodiments, a distinct DMR can be selected from the DMRs in Table 1, Table 2, Table 3, Table 4 or any combination thereof. In some cases, a method can comprise determining a methylation level in at least 10 distinct DMRs. In some cases, a method can comprise determining a methylation level in more than or less than about: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 distinct DMRs. In some cases, a method can comprise determining a methylation level in about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 distinct DMRs. In some cases, a method can comprise determining a methylation level in about: 1 to 250 DMRs, 1 to about 10 DMRs, 1 to about 25 DMRs, 10 to about 25 DMRs, 15 to about 30 DMRs 10 to about 50 DMRs, 20 to about 60 DMRs, 25 to about 80 DMRs, 50 to about 100 DMRs, 100 to about 150 DMRs, or about 150 to about 250 DMRs. In some embodiments, the methylation level of a sample can be hypermethylated, hypomethylated, or unchanged as compared to a reference methylation level.

In an embodiment, the plurality of distinct DMRs comprises at least 10 distinct DMRs. In an embodiment, the plurality of distinct DMRs comprises 2 to about 10 distinct DMRs. In an embodiment, the plurality of distinct DMRs comprises 5 to about 15 distinct DMRs. In an embodiment, the plurality of distinct DMRs comprises 3 to about 8 distinct DMRs. In an embodiment, the plurality of distinct DMRs comprises 4 to about 12 distinct DMRs. In an embodiment, the plurality of distinct DMRs comprises about 20 to about 30 distinct DMRs. In an embodiment, the plurality of distinct DMRs comprises about 10 to about 1000 distinct DMRs. In an embodiment, the plurality of distinct DMRs comprises about 10 to about 15 distinct DMRs. In an embodiment, the plurality of distinct DMRs comprises about 10 to about 50 distinct DMRs. In an embodiment, the plurality of distinct DMRs comprises about 10 to about 100 distinct DMRs. In an embodiment, the plurality of distinct DMRs comprises about 50 to about 100 distinct DMRs. In an embodiment, the plurality of distinct DMRs comprises about 100 to about 150 distinct DMRs. In an embodiment, the plurality of distinct DMRs comprises about 150 to about 200 distinct DMRs. In an embodiment, the plurality of distinct DMRs comprises about 200 to about 250 distinct DMRs. In an embodiment, the plurality of distinct DMRs comprises about 250 to about 300 distinct DMRs. In an embodiment, the plurality of distinct DMRs comprises about 300 to about 350 distinct DMRs. In an embodiment, the plurality of distinct DMRs comprises about 350 to about 400 distinct DMRs. In an embodiment, the plurality of distinct DMRs comprises about 400 to about 450 distinct DMRs. In an embodiment, the plurality of distinct DMRs comprises about 450 to about 500 distinct DMRs. In an embodiment, the plurality of distinct DMRs comprises about 500 to about 550 distinct DMRs. In an embodiment, the plurality of distinct DMRs comprises about 550 to about 600 distinct DMRs. In an embodiment, the plurality of distinct DMRs comprises about 600 to about 650 distinct DMRs. In an embodiment, the plurality of distinct DMRs comprises about 650 to about 700 distinct DMRs. In an embodiment, the plurality of distinct DMRs comprises about 700 to about 750 distinct DMRs. In an embodiment, the plurality of distinct DMRs comprises about 750 to about 800 distinct DMRs. In an embodiment, the plurality of distinct DMRs comprises about 800 to about 850 distinct DMRs. In an embodiment, the plurality of distinct DMRs comprises about 850 to about 900 distinct DMRs. In an embodiment, the plurality of distinct DMRs comprises about 900 to about 950 distinct DMRs. In an embodiment, the plurality of distinct DMRs comprises about 950 to about 1000 distinct DMRs.

Also disclosed herein are computer control systems that are programmed to implement methods described herein.

In some embodiments, the methods herein comprise the steps of detecting, comparing, determining, or any combination thereof. In some cases, the detecting can comprise executing a computer program on a computer. In some cases, the comparing can comprise executing a computer program on a computer. In some cases, the determining can comprise executing a computer program on a computer. In some embodiments, the detecting, the comparing, the determining, or any combination thereof employs a computer processor. In some embodiments, the detecting, the comparing, the determining, or any combination thereof employs computer readable memory. In some embodiments, the detecting, the comparing, the determining, or any combination thereof employs computer readable instructions on a computer readable memory. In some cases, the determining employs a computer processor for receiving and analyzing data comprising the presence or absence of a methylation level. In some cases, a computer program can be the same computer program. In some cases, a computer program can be a different computer program. For example, the detecting and comparing steps can use the same or different computer programs. In some cases, a computer can comprise a electronic for displaying data, a computation, an output, an input, or any combination thereof.

In some embodiments, computer systems can be used for analyzing DNA from a somatic cell DNA sample or blood cell DNA sample obtained from a subject. In some cases, the computer system can comprise: a) a device for receiving sequenced data, wherein the sequenced data comprises sequencing of at least one DMR of Table 1, Table 2, Table 3, Table 4 or any combination thereof; and b) a device for comparing the sequenced data with a control methylation level to determine the methylation level of the sequenced data. In some cases a computer system can be used to determine if a subject has or is at risk for developing a rheumatoid arthritis. In some cases, sequence data comprises data obtained from the sequencing of bisulfite converted DNA. In some cases, the methylation level comprises a hypermethylation or a hypomethylation of one or more DMRs from Table 1, Table 2, Table 3, Table 4 or any combination thereof.

In some cases, a device can be used for receiving an array and the data associated with an array. In some cases, a device can be used to compare the data from the array with a control. In some cases, the data from the array can be a methylation level. In some cases, a device can be a computer system.

In an embodiment, the plurality of distinct DMRs demonstrate an increase in DNA methylation. In an embodiment, the plurality of distinct DMRs demonstrate a decrease in DNA methylation.

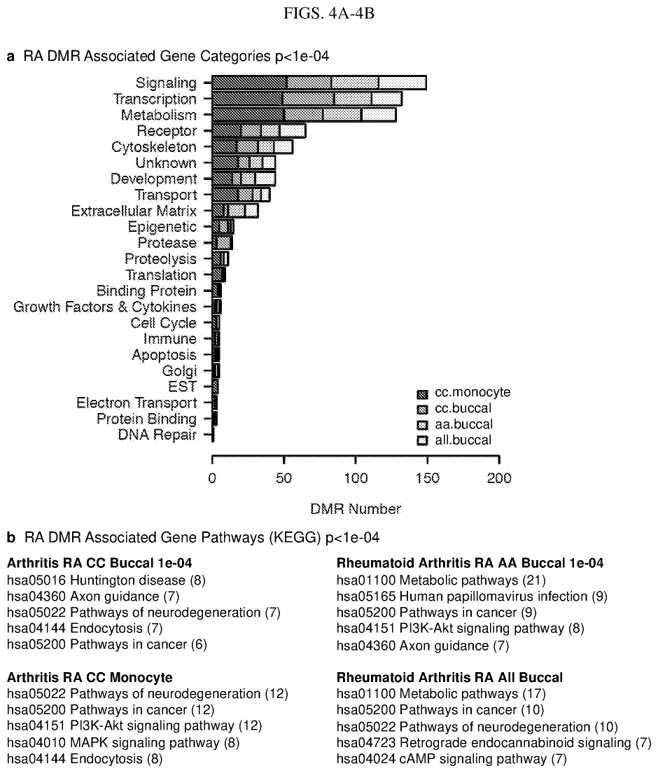

In an embodiment, the plurality of distinct DMRs are of a DMR associated gene category. Examples of DMR associated gene categories are presented in FIGS. 4A-4B.

In an embodiment, the DMR associated gene category is a signaling gene category. In an embodiment, the DMR associated gene category is a transcription gene category. In an embodiment, the DMR associated gene category is a metabolism gene category. In an embodiment, the DMR associated gene category is a receptor gene category. In an embodiment, the DMR associated gene category is a cytoskeleton gene category. In an embodiment, the DMR associated gene category is a development gene category. In an embodiment, the DMR associated gene category is a transport gene category. In an embodiment, the DMR associated gene category is an extracellular matrix gene category. In an embodiment, the DMR associated gene category is an epigenetic gene category. In an embodiment, the DMR associated gene category is a protease gene category. In an embodiment, the DMR associated gene category is a proteolysis gene category. In an embodiment, the DMR associated gene category is a translation gene category. In an embodiment, the DMR associated gene category is a binding protein gene category. In an embodiment, the DMR associated gene category is a growth factor/cytokine gene category. In an embodiment, the DMR associated gene category is a cell cycle gene category. In an embodiment, the DMR associated gene category is an immune gene category. In an embodiment, the DMR associated gene category is an apoptosis gene category. In an embodiment, the DMR associated gene category is a Golgi gene category. In an embodiment, the DMR associated gene category is an EST gene category. In an embodiment, the DMR associated gene category is an electron transport gene category. In an embodiment, the DMR associated gene category is a protein binding gene category. In an embodiment, the DMR associated gene category is a DNA repair gene category.

In an embodiment, the method comprises sequencing, wherein the sequencing comprises sequencing by synthesis, ion semiconductor sequencing, single molecule real time sequencing, nanopore sequencing, next-generation sequencing, or any combination thereof. Such sequencing procedures are well known in the art to the skilled person.

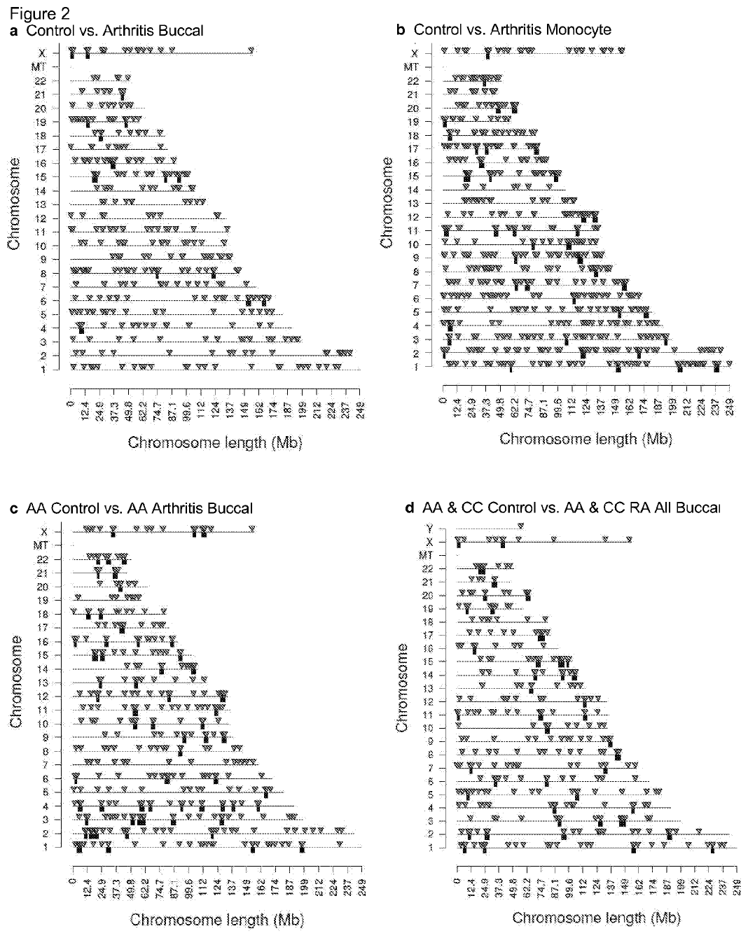

In an embodiment, the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes. Examples of chromosomal distribution of DMRs are presented in FIGS. 2A-2D.

In an embodiment, the DMRs that are determined and compared, individually, range from about 100 to about 17000 adjacent nucleotides or from about 10 to about 50,000 adjacent nucleotides. In some embodiments, the size of a DMR individually and independently can be about: 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 2000 nucleotides, 3000 nucleotides, 4000 nucleotides, 5000 nucleotides, 6000 nucleotides, 7000 nucleotides, 8000 nucleotides, 9000 nucleotides, 10000 nucleotides, 11000 nucleotides, 12000 nucleotides, 13000 nucleotides, 14000 nucleotides, 15000 nucleotides, 16000 nucleotides, 17000 nucleotides, 18000 nucleotides, 19000 nucleotides, 20000 nucleotides, 21000 nucleotides, 22000 nucleotides, 23000 nucleotides, 24000 nucleotides, 25000 nucleotides, 26000 nucleotides, 27000 nucleotides, 28000 nucleotides, 29000 nucleotides, 30000 nucleotides, 31000 nucleotides, 32000 nucleotides, 33000 nucleotides, 34000 nucleotides, 35000 nucleotides, 36000 nucleotides, 37000 nucleotides, 38000 nucleotides, 39000 nucleotides, 40000 nucleotides, 41000 nucleotides, 42000 nucleotides, 43000 nucleotides, 44000 nucleotides, 45000 nucleotides, 46000 nucleotides, 47000 nucleotides, 48000 nucleotides, 49000 nucleotides, 50000 nucleotides or greater than 50000 nucleotides. In some embodiments, the size of a DMR individually and independently can be about: 10 nucleotides to about 50,000 nucleotides, 10 nucleotides to about 1000 nucleotides, 100 nucleotides to about 1000 nucleotides, 500 nucleotides to about 10000 nucleotides, 1000 nucleotides to about 10000 nucleotides, 5000 nucleotides to about 20000 nucleotides, 10000 nucleotides to about 40000 nucleotides, or about 20000 nucleotides to about 50000 nucleotides.

In an embodiment, at least a plurality of the DMRs that are determined and compared comprise a CpG density of 1 to about 10 CpG per 100 nucleotides. In an embodiment, at least a plurality of the DMRs that are determined and compared comprise a CpG density of less than about 10 CpG per 100 nucleotides. In an embodiment, at least a plurality of the DMRs that are determined and compared comprise a CpG density of less than about 5 CpG per 100 nucleotides. In an embodiment, at least a plurality of the DMRs that are determined and compared comprise a CpG density of less than about 3 CpG per 100 nucleotides. In an embodiment, at least a plurality of the DMRs that are determined and compared comprise a CpG density of between 1 CpG and 3 CpG per 100 nucleotides. In some embodiments, a CpG density in a DMR can be between about: 1 CpG and 50 CpG per 100 nucleotides, 1 CpG and 40 CpG per 100 nucleotides, 1 CpG and 30 CpG per 100 nucleotides, 1 CpG and 20 CpG per 100 nucleotides, 2 CpG and 9 CpG per 100 nucleotides, 3 CpG and 8 CpG per 100 nucleotides, 4 CpG and 7 CpG per 100 nucleotides, 5 CpG and 15 CpG per 100 nucleotides, 10 CpG and 20 CpG per 100 nucleotides, or 15 CpG and 25 CpG per 100 nucleotides. In some embodiments, a CpG density in a DMR can be about: 1 CpG per 100 nucleotides, 2 CpG per 100 nucleotides, 3 CpG per 100 nucleotides, 4 CpG per 100 nucleotides, 5 CpG per 100 nucleotides, 6 CpG per 100 nucleotides, 7 CpG per 100 nucleotides, 8 CpG per 100 nucleotides, 9 CpG per 100 nucleotides, or 10 CpG per 100 nucleotides.

In an embodiment, at least about 50, 60, or 70 percent of the DMRs that are determined and compared are hypermethylated when compared, individually, to individual reference methylation levels of corresponding individual reference DMRs. In an embodiment, at least about 30, 40, or 50 percent of the DMRs that are determined and compared are hypomethylated when compared, individually, to individual reference methylation levels of corresponding individual reference DMRs.

In an embodiment, the human subject is a male subject. In an embodiment, the human subject is a female subject. In an embodiment, the human subject is a Caucasian subject. In an embodiment, the human subject is an African-American subject.

In an embodiment, the method further comprises determining a risk of the human subject having a disease or condition, wherein the determining is performed with a computer comprising a computer processor and computer readable memory comprising computer readable instructions.

In an embodiment, the disease or condition is rheumatoid arthritis. In an embodiment, the rheumatoid arthritis comprises a rheumatoid factor positive rheumatoid arthritis. In an embodiment, the rheumatoid arthritis comprises a rheumatoid factor negative rheumatoid arthritis. In an embodiment, the rheumatoid arthritis comprises juvenile rheumatoid arthritis. In an embodiment, the rheumatoid arthritis comprises juvenile idiopathic arthritis.

In an embodiment, the risk of the human subject further comprises determination of smoking status of the human subject, nutritional status of the human subject, alcohol consumption of the human subject, or any combination thereof.

In an embodiment, the method further comprises performing an additional analysis using a computer. In an embodiment, the additional analysis comprises a principle component analysis (PCA), a dendrogram analysis, a machine learning analysis, or any combination thereof.

In an embodiment, the additional analysis generates data points, and wherein the data points in the additional analysis are grouped into two spatially distinct categories comprising a first category and a second category. In an embodiment, the first category indicates the human subject is at increased risk of having a disease or condition and In an embodiment, the second category indicates the human subject is not at increased risk of having the disease or condition.

In an embodiment, the method further comprises treating the human subject. In an embodiment, the treating comprises administering a medication for treatment of rheumatoid arthritis. In an embodiment, the medication is an antibody. In an embodiment, the medication is an anti-inflammatory. In an embodiment, the anti-inflammatory is a steroid. In an embodiment, the steroid comprises a corticosteroid or a salt thereof. In an embodiment, the anti-inflammatory is a non-steroidal anti-inflammatory drug (NSAID). In an embodiment, the NSAID comprises ibuprofen, naproxen sodium, or a salt thereof.

In an embodiment, the medication comprises methotrexate or a salt thereof. In an embodiment, the medication comprises leflunomide or a salt thereof. In an embodiment, the medication comprises hydroxychloroquine or a salt thereof. In an embodiment, the medication comprises sulfasalazine or a salt thereof. In an embodiment, the medication comprises abatacept. In an embodiment, the medication comprises adalimumab. In an embodiment, the medication comprises anakinra. In an embodiment, the medication comprises certolizumab. In an embodiment, the medication comprises etanercept. In an embodiment, the medication comprises golimumab. In an embodiment, the medication comprises infliximab. In an embodiment, the medication comprises rituximab. In an embodiment, the medication comprises sarilumab. In an embodiment, the medication comprises tocilizumab.

In an embodiment, the treating comprises administering a medication for preventative treatment of rheumatoid arthritis. In an embodiment, the medication is an antibody. In an embodiment, the medication is an anti-inflammatory. In an embodiment, the anti-inflammatory is a steroid. In an embodiment, the steroid comprises a corticosteroid or a salt thereof. In an embodiment, the anti-inflammatory is a non-steroidal anti-inflammatory drug (NSAID). In an embodiment, the NSAID comprises ibuprofen, naproxen sodium, or a salt thereof.

In an embodiment, the medication comprises methotrexate or a salt thereof. In an embodiment, the medication comprises leflunomide or a salt thereof. In an embodiment, the medication comprises hydroxychloroquine or a salt thereof. In an embodiment, the medication comprises sulfasalazine or a salt thereof.

In an embodiment, the medication comprises abatacept. In an embodiment, the medication comprises adalimumab. In an embodiment, the medication comprises anakinra. In an embodiment, the medication comprises certolizumab. In an embodiment, the medication comprises etanercept. In an embodiment, the medication comprises golimumab. In an embodiment, the medication comprises infliximab. In an embodiment, the medication comprises rituximab. In an embodiment, the medication comprises sarilumab. In an embodiment, the medication comprises tocilizumab.

In some embodiments, methods described herein can comprise administering a therapy (e.g., treatment) to a subject in need thereof, for example a subject having a rheumatoid arthritis.

In some embodiments, the terms “administer,” “administering”, “administration,” and the like, as used herein, can refer to methods that can be used to deliver therapies described herein. In some cases, delivery can include injection, inhalation, catheterization, gastrostomy tube administration, intravenous administration, intraosseous administration, ocular administration, otic administration, topical administration, transdermal administration, oral administration, rectal administration, nasal administration, intravaginal administration, intracavernous administration, intracerebral administration, transurethral administration, buccal administration, sublingual administration, intrapenile drug delivery, subcutaneous administration, or a combination thereof. Delivery can include a parenchymal injection, an intra-thecal injection, an intra-ventricular injection, or an intra-cisternal injection. A therapy provided herein can be administered by any method. In some cases, a medical professional can administer the therapy described herein.

Administration of a therapy disclosed herein can be performed for a treatment duration of at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, or more consecutive or nonconsecutive days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days.

Administration or application of a therapy disclosed herein can be performed for a treatment duration of at least about 1 week, at least about 2 weeks, at least about 3 weeks, at least about 4 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 1 year, at least about 2 years, or for life. In some embodiments, administering can be performed for about: 1 day to about 8 days, 1 week to about 5 weeks, 1 month to about 12 months, or 1 year to about 3 years.

Administration can be performed repeatedly over a lifetime of a subject, such as once a month or once a year for the lifetime of a subject.

Administration or application of a therapy disclosed herein can be performed at least 1, 2, 3, 4, 5, 6, 7, 8, 9, 10, 11, 12, 13, 14, 15, 16, 17, 18, 19, 20, 21, 22, 23, or 24 times a in a 24 hour period. In some cases, administration or application of a therapy disclosed herein can be performed continuously throughout a 24 hour period. In some cases, administration or application of a therapy disclosed herein can be performed at least 1, 2, 3, 4, 5, 6, 7, 8, 9, 10, or more times a week. In some cases, administration or application of a therapy disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more times a month. In some cases, a therapy can be administered as a single dose or as divided doses. In some cases, a therapy described herein can be administered at a first time point and a second time point.

In some cases, a therapy herein can be administered at a dose of about 0.0001 grams to about 1000 grams. In some cases a therapy herein can be administered at a dose of about 1 mg to about 1 gram. In some cases, a therapy herein can be administered at a dose of about: 10 μg, 100 μg, 500 μg 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or 100 mg. In some cases, a therapy herein can be a pharmaceutical composition. In some cases, a therapy or a pharmaceutical composition can be in unit dose form.

In some cases, a therapy can be administered with an excipient, a carrier, a diluent or any combination thereof. In some cases, a carrier, a diluent, or both, can comprise water, saline, or any pharmaceutically acceptable carrier and/or diluent. In some cases, a diluent can comprise a pH buffer. In some cases, an excipient, carrier, or diluent can comprise water.

In an embodiment, the method further comprises transmitting data, a result, or both, via an electronic communication medium.

In an illustrative aspect, a second method is provided. The method comprises obtaining a sample of a somatic cell from a human subject; isolating deoxyribonucleic acid (DNA) from the sample, thereby obtaining isolated DNA; detecting and determining a methylation level of a differential DNA methylation region (DMR) comprised in the isolated DNA; and comparing the methylation level of the DMR to a reference methylation level of a corresponding reference DMR; wherein the comparing comprises employing a computer comprising a computer processor and computer readable memory comprising computer readable instructions contained thereon, wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and wherein a plurality of determined DMRs are sufficient to determine, from a process comprising the comparing and employing a computer, whether the human subject has or is at increased risk of having rheumatoid arthritis.

The previously described embodiments of the first method are applicable to the second method as described herein.

In an illustrative aspect, a first kit is provided. The kit comprises at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence selected from the DMRs in Table 1, the DMRs in Table 2, the DMRs in Table 3, the DMRs in Table 4, and any combination thereof; and a container.

In an embodiment, the distinct primers or pairs of primers each further comprise a unique barcode. In an embodiment, the distinct primers or pairs of primers are not bound to an array or a microarray. In an embodiment, the distinct primers or pairs of primers are bound to an array or a microarray. In an embodiment, the distinct primers or pairs of primers comprise DNA.

In an embodiment, the distinct DMR sequences are selected from the DMRs in Table 1. In an embodiment, the distinct DMR sequences are selected from the DMRs in Table 2. In an embodiment, the distinct DMR sequences are selected from the DMRs in Table 3. In an embodiment, the distinct DMR sequences are selected from the DMRs in Table 4.

In an embodiment, the distinct DMR sequences comprises about 50 to about 100 DMRs. In an embodiment, the distinct DMR sequences comprises about 100 to about 150 DMRs. In an embodiment, the distinct DMR sequences comprises about 150 to about 200 DMRs. In an embodiment, the distinct DMR sequences comprises about 200 to about 250 DMRs. In an embodiment, the distinct DMR sequences comprises about 250 to about 300 DMRs. In an embodiment, the distinct DMR sequences comprises about 300 to about 350 DMRs. In an embodiment, the distinct DMR sequences comprises about 350 to about 400 DMRs. In an embodiment, the distinct DMR sequences comprises about 400 to about 450 DMRs. In an embodiment, the distinct DMR sequences comprises about 450 to about 500 DMRs. In an embodiment, the distinct DMR sequences comprises about 500 to about 550 DMRs. In an embodiment, the distinct DMR sequences comprises about 550 to about 600 DMRs. In an embodiment, the distinct DMR sequences comprises about 600 to about 650 DMRs. In an embodiment, the distinct DMR sequences comprises about 650 to about 700 DMRs. In an embodiment, the distinct DMR sequences comprises about 700 to about 750 DMRs. In an embodiment, the distinct DMR sequences comprises about 750 to about 800 DMRs. In an embodiment, the distinct DMR sequences comprises about 800 to about 850 DMRs. In an embodiment, the distinct DMR sequences comprises about 850 to about 900 DMRs. In an embodiment, the distinct DMR sequences comprises about 900 to about 950 DMRs. In an embodiment, the distinct DMR sequences comprises about 950 to about 1000 DMRs.

In an embodiment, the distinct DMR sequences are of a DMR associated gene category. In an embodiment, the DMR associated gene category is a signaling gene category. In an embodiment, the DMR associated gene category is a transcription gene category. In an embodiment, the DMR associated gene category is a metabolism gene category. In an embodiment, the DMR associated gene category is a receptor gene category. In an embodiment, the DMR associated gene category is a cytoskeleton gene category. In an embodiment, the DMR associated gene category is a development gene category. In an embodiment, the DMR associated gene category is a transport gene category. In an embodiment, the DMR associated gene category is an extracellular matrix gene category. In an embodiment, the DMR associated gene category is an epigenetic gene category. In an embodiment, the DMR associated gene category is a protease gene category. In an embodiment, the DMR associated gene category is a proteolysis gene category. In an embodiment, the DMR associated gene category is a translation gene category. In an embodiment, the DMR associated gene category is a binding protein gene category. In an embodiment, the DMR associated gene category is a growth factor/cytokine gene category. In an embodiment, the DMR associated gene category is a cell cycle gene category. In an embodiment, the DMR associated gene category is an immune gene category. In an embodiment, the DMR associated gene category is an apoptosis gene category. In an embodiment, the DMR associated gene category is a Golgi gene category. In an embodiment, the DMR associated gene category is an EST gene category. In an embodiment, the DMR associated gene category is an electron transport gene category. In an embodiment, the DMR associated gene category is a protein binding gene category. In an embodiment, the DMR associated gene category is a DNA repair gene category.

In an illustrative aspect, a second kit is provided. The kit comprises at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct probes, each distinct probe complementary to a distinct DMR sequence selected from the DMRs in Table 1, the DMRs in Table 2, the DMRs in Table 3, the DMRs in Table 4, and any combination thereof; and a container.

The previously described embodiments of the first kit are applicable to the second kit as described herein. In an embodiment, the distinct probes further comprise at least one of fluorophore, a chromophore, a barcode, or any combination thereof. In an embodiment, each probe comprises a unique fluorophore, a chromophore, barcode, or any combination thereof.

In an illustrative aspect, a third method is provided. The method comprises: obtaining a sample of a somatic cell from a human subject; isolating deoxyribonucleic acid (DNA) from the sample, thereby obtaining isolated DNA; fragmenting the DNA; isolating fragmented methylated DNA; detecting and determining a methylation level of a differential DNA methylation region (DMR) comprised in the isolated fragmented methylated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises i) amplifying the isolated fragmented methylated DNA, ii) sequencing the isolated fragmented methylated DNA, an amplicon thereof, or both, or iii) employing an array, or iv) any combination thereof, wherein a plurality of distinct DMRs are detected and compared, wherein the plurality comprises at least 10 distinct DMRs.

The previously described embodiments of the first method and the second method are applicable to the third method as described herein. In an embodiment, the fragmenting is performed via sonication. In an embodiment, the isolating the fragmented methylated DNA comprises methylated DNA immunoprecipitation (MeDIP).

In an illustrative aspect, a fourth method is provided. The method comprises: obtaining a sample of a somatic cell from a human subject; isolating deoxyribonucleic acid (DNA) from the sample, thereby obtaining isolated DNA; fragmenting the DNA; isolating fragmented methylated DNA; detecting and determining a methylation level of a differential DNA methylation region (DMR) comprised in the isolated fragmented methylated DNA; and comparing the methylation level of the DMR to a reference methylation level of a corresponding reference DMR; wherein the comparing comprises comparing employing a computer comprising a computer processor and computer readable memory comprising computer readable instructions contained thereon, wherein the detecting and determining comprises i) amplifying the isolated fragmented methylated DNA, ii) sequencing the isolated fragmented methylated DNA, an amplicon thereof, or both, or iii) employing an array, or iv) any combination thereof; and wherein a plurality of determined DMRs are sufficient to determine, from a process comprising the comparing and employing a computer, whether the human subject has or is at increased risk of having rheumatoid arthritis.

The previously described embodiments of the first method, the second method, and the third method are applicable to the fourth method as described herein. In an embodiment, the fragmenting is performed via sonication. In an embodiment, the isolating the fragmented methylated DNA comprises methylated DNA immunoprecipitation (MeDIP).

The following numbered embodiments are contemplated and are non-limiting:

1. A method comprising:
obtaining a sample of a somatic cell from a human subject;
isolating deoxyribonucleic acid (DNA) from the sample, thereby obtaining isolated DNA; detecting and determining a methylation level of a differential DNA methylation region (DMR) comprised in the isolated DNA; and
comparing the methylation level of the DMR to a reference methylation level of a corresponding reference DMR;
wherein the comparing comprises comparing employing a computer comprising a computer processor and computer readable memory comprising computer readable instructions contained thereon,
wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
wherein a plurality of distinct DMRs are detected and compared, wherein the plurality comprises at least 10 distinct DMRs.

2. The method of clause 1, any other suitable clause, or any combination of suitable clauses, wherein the somatic cell comprises a buccal cell.

3. The method of clause 1, any other suitable clause, or any combination of suitable clauses, wherein the somatic cell consists essentially of a buccal cell.

4. The method of clause 1, any other suitable clause, or any combination of suitable clauses, wherein the somatic cell consists of a buccal cell.

5. The method of clause 1, any other suitable clause, or any combination of suitable clauses, wherein the somatic cell comprises a blood monocyte cell.

6. The method of clause 5, any other suitable clause, or any combination of suitable clauses, wherein the blood monocyte cell is a purified blood monocyte cell.

7. The method of clause 1, any other suitable clause, or any combination of suitable clauses, wherein the somatic cell consists essentially of a blood monocyte cell.

8. The method of clause 7, any other suitable clause, or any combination of suitable clauses, wherein the blood monocyte cell is a purified blood monocyte cell.

9. The method of clause 1, any other suitable clause, or any combination of suitable clauses, wherein the somatic cell consists of a blood monocyte cell.

10. The method of clause 9, any other suitable clause, or any combination of suitable clauses, wherein the blood monocyte cell is a purified blood monocyte cell.

11. The method of clause 1, any other suitable clause, or any combination of suitable clauses, wherein the somatic cell comprises a muscle cell, a blood cell, a skin cell, a nerve cell, or any combination thereof.

12. The method of clause 1, any other suitable clause, or any combination of suitable clauses, wherein the somatic cell comprises an immune cell.

13. The method of clause 1, any other suitable clause, or any combination of suitable clauses, wherein the somatic cell comprises a muscle cell.

14. The method of clause 1, any other suitable clause, or any combination of suitable clauses, wherein the somatic cell comprises a blood cell.

15. The method of clause 1, any other suitable clause, or any combination of suitable clauses, wherein the somatic cell comprises a skin cell.

16. The method of clause 1, any other suitable clause, or any combination of suitable clauses, wherein the somatic cell comprises a nerve cell.

17. The method of clause 1, any other suitable clause, or any combination of suitable clauses, wherein the plurality of distinct DMRs are selected from the DMRs in Table 1, the DMRs in Table 2, the DMRs in Table 3, the DMRs in Table 4, and any combination thereof.

18. The method of clause 1, any other suitable clause, or any combination of suitable clauses, wherein the plurality of distinct DMRs are selected from the DMRs in Table 1.

19. The method of clause 1, any other suitable clause, or any combination of suitable clauses, wherein the plurality of distinct DMRs are selected from the DMRs in Table 2.

20. The method of clause 1, any other suitable clause, or any combination of suitable clauses, wherein the plurality of distinct DMRs are selected from the DMRs in Table 3.

21. The method of clause 1, any other suitable clause, or any combination of suitable clauses, wherein the plurality of distinct DMRs are selected from the DMRs in Table 4.

22. The method of clause 1, any other suitable clause, or any combination of suitable clauses, wherein the plurality of distinct DMRs comprises at least 10 distinct DMRs.

23. The method of clause 1, any other suitable clause, or any combination of suitable clauses, wherein the plurality of distinct DMRs comprises 2 to about 10 distinct DMRs.

24. The method of clause 1, any other suitable clause, or any combination of suitable clauses, wherein the plurality of distinct DMRs comprises 5 to about 15 distinct DMRs.

25. The method of clause 1, any other suitable clause, or any combination of suitable clauses, wherein the plurality of distinct DMRs comprises 3 to about 8 distinct DMRs.

26. The method of clause 1, any other suitable clause, or any combination of suitable clauses, wherein the plurality of distinct DMRs comprises 4 to about 12 distinct DMRs.

27. The method of clause 1, any other suitable clause, or any combination of suitable clauses, wherein the plurality of distinct DMRs comprises about 20 to about 30 distinct DMRs.

28. The method of clause 1, any other suitable clause, or any combination of suitable clauses, wherein the plurality of distinct DMRs comprises about 10 to about 1000 distinct DMRs.

29. The method of clause 1, any other suitable clause, or any combination of suitable clauses, wherein the plurality of distinct DMRs comprises about 10 to about 15 distinct DMRs.

30. The method of clause 1, any other suitable clause, or any combination of suitable clauses, wherein the plurality of distinct DMRs comprises about 10 to about 50 distinct DMRs.

31. The method of clause 1, any other suitable clause, or any combination of suitable clauses, wherein the plurality of distinct DMRs comprises about 10 to about 100 distinct DMRs.

32. The method of clause 1, any other suitable clause, or any combination of suitable clauses, wherein the plurality of distinct DMRs comprises about 50 to about 100 distinct DMRs.

33. The method of clause 1, any other suitable clause, or any combination of suitable clauses, wherein the plurality of distinct DMRs comprises about 100 to about 150 distinct DMRs.

34. The method of clause 1, any other suitable clause, or any combination of suitable clauses, wherein the plurality of distinct DMRs comprises about 150 to about 200 distinct DMRs.

35. The method of clause 1, any other suitable clause, or any combination of suitable clauses, wherein the plurality of distinct DMRs comprises about 200 to about 250 distinct DMRs.

36. The method of clause 1, any other suitable clause, or any combination of suitable clauses, wherein the plurality of distinct DMRs comprises about 250 to about 300 distinct DMRs.

37. The method of clause 1, any other suitable clause, or any combination of suitable clauses, wherein the plurality of distinct DMRs comprises about 300 to about 350 distinct DMRs.

38. The method of clause 1, any other suitable clause, or any combination of suitable clauses, wherein the plurality of distinct DMRs comprises about 350 to about 400 distinct DMRs.

39. The method of clause 1, any other suitable clause, or any combination of suitable clauses, wherein the plurality of distinct DMRs comprises about 400 to about 450 distinct DMRs.

40. The method of clause 1, any other suitable clause, or any combination of suitable clauses, wherein the plurality of distinct DMRs comprises about 450 to about 500 distinct DMRs.

41. The method of clause 1, any other suitable clause, or any combination of suitable clauses, wherein the plurality of distinct DMRs comprises about 500 to about 550 distinct DMRs.

42. The method of clause 1, any other suitable clause, or any combination of suitable clauses, wherein the plurality of distinct DMRs comprises about 550 to about 600 distinct DMRs.

43. The method of clause 1, any other suitable clause, or any combination of suitable clauses, wherein the plurality of distinct DMRs comprises about 600 to about 650 distinct DMRs.

44. The method of clause 1, any other suitable clause, or any combination of suitable clauses, wherein the plurality of distinct DMRs comprises about 650 to about 700 distinct DMRs.

45. The method of clause 1, any other suitable clause, or any combination of suitable clauses, wherein the plurality of distinct DMRs comprises about 700 to about 750 distinct DMRs.

46. The method of clause 1, any other suitable clause, or any combination of suitable clauses, wherein the plurality of distinct DMRs comprises about 750 to about 800 distinct DMRs.

47. The method of clause 1, any other suitable clause, or any combination of suitable clauses, wherein the plurality of distinct DMRs comprises about 800 to about 850 distinct DMRs.

48. The method of clause 1, any other suitable clause, or any combination of suitable clauses, wherein the plurality of distinct DMRs comprises about 850 to about 900 distinct DMRs.

49. The method of clause 1, any other suitable clause, or any combination of suitable clauses, wherein the plurality of distinct DMRs comprises about 900 to about 950 distinct DMRs.

50. The method of clause 1, any other suitable clause, or any combination of suitable clauses, wherein the plurality of distinct DMRs comprises about 950 to about 1000 distinct DMRs.

51. The method of clause 1, any other suitable clause, or any combination of suitable clauses, wherein the plurality of distinct DMRs demonstrate an increase in DNA methylation.

52. The method of clause 1, any other suitable clause, or any combination of suitable clauses, wherein the plurality of distinct DMRs demonstrate a decrease in DNA methylation.

53. The method of clause 1, any other suitable clause, or any combination of suitable clauses, wherein the plurality of distinct DMRs are of a DMR associated gene category.

54. The method of clause 53, any other suitable clause, or any combination of suitable clauses, wherein the DMR associated gene category is a signaling gene category.

55. The method of clause 53, any other suitable clause, or any combination of suitable clauses, wherein the DMR associated gene category is a transcription gene category.

56. The method of clause 53, any other suitable clause, or any combination of suitable clauses, wherein the DMR associated gene category is a metabolism gene category.

57. The method of clause 53, any other suitable clause, or any combination of suitable clauses, wherein the DMR associated gene category is a receptor gene category.

58. The method of clause 53, any other suitable clause, or any combination of suitable clauses, wherein the DMR associated gene category is a cytoskeleton gene category.

59. The method of clause 53, any other suitable clause, or any combination of suitable clauses, wherein the DMR associated gene category is a development gene category.

60. The method of clause 53, any other suitable clause, or any combination of suitable clauses, wherein the DMR associated gene category is a transport gene category.

61. The method of clause 53, any other suitable clause, or any combination of suitable clauses, wherein the DMR associated gene category is an extracellular matrix gene category.

62. The method of clause 53, any other suitable clause, or any combination of suitable clauses, wherein the DMR associated gene category is an epigenetic gene category.

63. The method of clause 53, any other suitable clause, or any combination of suitable clauses, wherein the DMR associated gene category is a protease gene category.

64. The method of clause 53, any other suitable clause, or any combination of suitable clauses, wherein the DMR associated gene category is a proteolysis gene category.

65. The method of clause 53, any other suitable clause, or any combination of suitable clauses, wherein the DMR associated gene category is a translation gene category.

66. The method of clause 53, any other suitable clause, or any combination of suitable clauses, wherein the DMR associated gene category is a binding protein gene category.

67. The method of clause 53, any other suitable clause, or any combination of suitable clauses, wherein the DMR associated gene category is a growth factor/cytokine gene category.

68. The method of clause 53, any other suitable clause, or any combination of suitable clauses, wherein the DMR associated gene category is a cell cycle gene category.

69. The method of clause 53, any other suitable clause, or any combination of suitable clauses, wherein the DMR associated gene category is an immune gene category.

70. The method of clause 53, any other suitable clause, or any combination of suitable clauses, wherein the DMR associated gene category is an apoptosis gene category.

71. The method of clause 53, any other suitable clause, or any combination of suitable clauses, wherein the DMR associated gene category is a Golgi gene category.

72. The method of clause 53, any other suitable clause, or any combination of suitable clauses, wherein the DMR associated gene category is an EST gene category.

73. The method of clause 53, any other suitable clause, or any combination of suitable clauses, wherein the DMR associated gene category is an electron transport gene category.

74. The method of clause 53, any other suitable clause, or any combination of suitable clauses, wherein the DMR associated gene category is a protein binding gene category.

75. The method of clause 53, any other suitable clause, or any combination of suitable clauses, wherein the DMR associated gene category is a DNA repair gene category.

76. The method of clause 1, any other suitable clause, or any combination of suitable clauses, comprising sequencing, wherein the sequencing comprises sequencing by synthesis, ion semiconductor sequencing, single molecule real time sequencing, nanopore sequencing, next-generation sequencing, or any combination thereof.

77. The method of clause 1, any other suitable clause, or any combination of suitable clauses, wherein the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.

78. The method of clause 1, any other suitable clause, or any combination of suitable clauses, wherein the DMRs that are determined and compared, individually, range from about 100 to about 17000 adjacent nucleotides.

79. The method of clause 1, any other suitable clause, or any combination of suitable clauses, wherein at least a plurality of the DMRs that are determined and compared comprise a CpG density of 1 to about 10 CpG per 100 nucleotides.

80. The method of clause 79, any other suitable clause, or any combination of suitable clauses, wherein at least a plurality of the DMRs that are determined and compared comprise a CpG density of less than about 10 CpG per 100 nucleotides.

81. The method of clause 79, any other suitable clause, or any combination of suitable clauses, wherein at least a plurality of the DMRs that are determined and compared comprise a CpG density of less than about 5 CpG per 100 nucleotides.

82. The method of clause 79, any other suitable clause, or any combination of suitable clauses, wherein at least a plurality of the DMRs that are determined and compared comprise a CpG density of less than about 3 CpG per 100 nucleotides.

83. The method of clause 79, any other suitable clause, or any combination of suitable clauses, wherein at least a plurality of the DMRs that are determined and compared comprise a CpG density of between 1 CpG and 3 CpG per 100 nucleotides.

84. The method of clause 1, any other suitable clause, or any combination of suitable clauses, wherein at least about 50, 60, or 70 percent of the DMRs that are determined and compared are hypermethylated when compared, individually, to individual reference methylation levels of corresponding individual reference DMRs.

85. The method of clause 1, any other suitable clause, or any combination of suitable clauses, wherein at least about 30, 40, or 50 percent of the DMRs that are determined and compared are hypomethylated when compared, individually, to individual reference methylation levels of corresponding individual reference DMRs.

86. The method of clause 1, any other suitable clause, or any combination of suitable clauses, wherein the human subject is a male subject.

87. The method of clause 1, any other suitable clause, or any combination of suitable clauses, wherein the human subject is a female subject.

88. The method of clause 1, any other suitable clause, or any combination of suitable clauses, wherein the human subject is a Caucasian subject.

89. The method of clause 1, any other suitable clause, or any combination of suitable clauses, wherein the human subject is an African-American subject.

90. The method of clause 1, any other suitable clause, or any combination of suitable clauses, wherein the method further comprises determining a risk of the human subject having a disease or condition, wherein the determining is performed with a computer comprising a computer processor and computer readable memory comprising computer readable instructions.

91. The method of clause 90, any other suitable clause, or any combination of suitable clauses, wherein the disease or condition is rheumatoid arthritis.

92. The method of clause 91, any other suitable clause, or any combination of suitable clauses, wherein the rheumatoid arthritis comprises a rheumatoid factor positive rheumatoid arthritis.

93. The method of clause 91, any other suitable clause, or any combination of suitable clauses, wherein the rheumatoid arthritis comprises a rheumatoid factor negative rheumatoid arthritis.

94. The method of clause 91, any other suitable clause, or any combination of suitable clauses, wherein the rheumatoid arthritis comprises juvenile rheumatoid arthritis.

95. The method of clause 91, any other suitable clause, or any combination of suitable clauses, wherein the rheumatoid arthritis comprises juvenile idiopathic arthritis.

96. The method of clause 90, any other suitable clause, or any combination of suitable clauses, wherein the risk of the human subject further comprises determination of smoking status of the human subject, nutritional status of the human subject, alcohol consumption of the human subject, or any combination thereof.

97. The method of clause 1, any other suitable clause, or any combination of suitable clauses, further comprising performing an additional analysis using a computer.

98. The method of clause 97, any other suitable clause, or any combination of suitable clauses, wherein the additional analysis comprises a principle component analysis (PCA), a dendrogram analysis, a machine learning analysis, or any combination thereof.

99. The method of clause 97, any other suitable clause, or any combination of suitable clauses, wherein the additional analysis generates data points, and wherein the data points in the additional analysis are grouped into two spatially distinct categories comprising a first category and a second category.

100. The method of clause 99, any other suitable clause, or any combination of suitable clauses, wherein the first category indicates the human subject is at increased risk of having a disease or condition and wherein the second category indicates the human subject is not at increased risk of having the disease or condition.

101. The method of clause 1, any other suitable clause, or any combination of suitable clauses, further comprising treating the human subject.

102. The method of clause 101, any other suitable clause, or any combination of suitable clauses, wherein the treating comprises administering a medication for treatment of rheumatoid arthritis.

103. The method of clause 102, any other suitable clause, or any combination of suitable clauses, wherein the medication is an antibody.

104. The method of clause 102, any other suitable clause, or any combination of suitable clauses, wherein the medication is an anti-inflammatory.

105. The method of clause 104, any other suitable clause, or any combination of suitable clauses, wherein the anti-inflammatory is a steroid.

106. The method of clause 105, any other suitable clause, or any combination of suitable clauses, wherein the steroid comprises a corticosteroid or a salt thereof.

107. The method of clause 104, any other suitable clause, or any combination of suitable clauses, wherein the anti-inflammatory is a non-steroidal anti-inflammatory drug (NSAID).

108. The method of clause 107, any other suitable clause, or any combination of suitable clauses, wherein the NSAID comprises ibuprofen, naproxen sodium, or a salt thereof.

109. The method of clause 102, any other suitable clause, or any combination of suitable clauses, wherein the medication comprises methotrexate or a salt thereof.

110. The method of clause 102, any other suitable clause, or any combination of suitable clauses, wherein the medication comprises leflunomide or a salt thereof.

111. The method of clause 102, any other suitable clause, or any combination of suitable clauses, wherein the medication comprises hydroxychloroquine or a salt thereof.

112. The method of clause 102, any other suitable clause, or any combination of suitable clauses, wherein the medication comprises sulfasalazine or a salt thereof.

113. The method of clause 102, any other suitable clause, or any combination of suitable clauses, wherein the medication comprises abatacept.

114. The method of clause 102, any other suitable clause, or any combination of suitable clauses, wherein the medication comprises adalimumab.

115. The method of clause 102, any other suitable clause, or any combination of suitable clauses, wherein the medication comprises anakinra.

116. The method of clause 102, any other suitable clause, or any combination of suitable clauses, wherein the medication comprises certolizumab.

117. The method of clause 102, any other suitable clause, or any combination of suitable clauses, wherein the medication comprises etanercept.

118. The method of clause 102, any other suitable clause, or any combination of suitable clauses, wherein the medication comprises golimumab.

119. The method of clause 102, any other suitable clause, or any combination of suitable clauses, wherein the medication comprises infliximab.

120. The method of clause 102, any other suitable clause, or any combination of suitable clauses, wherein the medication comprises rituximab.

121. The method of clause 102, any other suitable clause, or any combination of suitable clauses, wherein the medication comprises sarilumab.

122. The method of clause 102, any other suitable clause, or any combination of suitable clauses, wherein the medication comprises tocilizumab.

123. The method of clause 101, any other suitable clause, or any combination of suitable clauses, wherein the treating comprises administering a medication for preventative treatment of rheumatoid arthritis.

124. The method of clause 123, any other suitable clause, or any combination of suitable clauses, wherein the medication is an antibody.

125. The method of clause 123, any other suitable clause, or any combination of suitable clauses, wherein the medication is an anti-inflammatory.

126. The method of clause 125, any other suitable clause, or any combination of suitable clauses, wherein the anti-inflammatory is a steroid.

127. The method of clause 126, any other suitable clause, or any combination of suitable clauses, wherein the steroid comprises a corticosteroid or a salt thereof.

128. The method of clause 125, any other suitable clause, or any combination of suitable clauses, wherein the anti-inflammatory is a non-steroidal anti-inflammatory drug (NSAID).

129. The method of clause 128, any other suitable clause, or any combination of suitable clauses, wherein the NSAID comprises ibuprofen, naproxen sodium, or a salt thereof.

130. The method of clause 123, any other suitable clause, or any combination of suitable clauses, wherein the medication comprises methotrexate or a salt thereof.

131. The method of clause 123, any other suitable clause, or any combination of suitable clauses, wherein the medication comprises leflunomide or a salt thereof.

132. The method of clause 123, any other suitable clause, or any combination of suitable clauses, wherein the medication comprises hydroxychloroquine or a salt thereof.

133. The method of clause 123, any other suitable clause, or any combination of suitable clauses, wherein the medication comprises sulfasalazine or a salt thereof.

134. The method of clause 123, any other suitable clause, or any combination of suitable clauses, wherein the medication comprises abatacept.

135. The method of clause 123, any other suitable clause, or any combination of suitable clauses, wherein the medication comprises adalimumab.

136. The method of clause 123, any other suitable clause, or any combination of suitable clauses, wherein the medication comprises anakinra.

137. The method of clause 123, any other suitable clause, or any combination of suitable clauses, wherein the medication comprises certolizumab.

138. The method of clause 123, any other suitable clause, or any combination of suitable clauses, wherein the medication comprises etanercept.

139. The method of clause 123, any other suitable clause, or any combination of suitable clauses, wherein the medication comprises golimumab.

140. The method of clause 123, any other suitable clause, or any combination of suitable clauses, wherein the medication comprises infliximab.

141. The method of clause 123, any other suitable clause, or any combination of suitable clauses, wherein the medication comprises rituximab.

142. The method of clause 123, any other suitable clause, or any combination of suitable clauses, wherein the medication comprises sarilumab.

143. The method of clause 123, any other suitable clause, or any combination of suitable clauses, wherein the medication comprises tocilizumab.

144. The method of clause 1, any other suitable clause, or any combination of suitable clauses, further comprising transmitting data, a result, or both, via an electronic communication medium.

145. A method comprising:
obtaining a sample of a somatic cell from a human subject;
isolating deoxyribonucleic acid (DNA) from the sample, thereby obtaining isolated DNA;
detecting and determining a methylation level of a differential DNA methylation region (DMR) comprised in the isolated DNA; and
comparing the methylation level of the DMR to a reference methylation level of a corresponding reference DMR;
wherein the comparing comprises employing a computer comprising a computer processor and computer readable memory comprising computer readable instructions contained thereon,
wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
wherein a plurality of determined DMRs are sufficient to determine, from a process comprising the comparing and employing a computer, whether the human subject has or is at increased risk of having rheumatoid arthritis.

146. The method of clause 145, any other suitable clause, or any combination of suitable clauses, wherein the somatic cell comprises a buccal cell.

147. The method of clause 145, any other suitable clause, or any combination of suitable clauses, wherein the somatic cell consists essentially of a buccal cell.

148. The method of clause 145, any other suitable clause, or any combination of suitable clauses, wherein the somatic cell consists of a buccal cell.

149. The method of clause 145, any other suitable clause, or any combination of suitable clauses, wherein the somatic cell comprises a blood monocyte cell.

150. The method of clause 149, any other suitable clause, or any combination of suitable clauses, wherein the blood monocyte cell is a purified blood monocyte cell.

151. The method of clause 145, any other suitable clause, or any combination of suitable clauses, wherein the somatic cell consists essentially of a blood monocyte cell.

152. The method of clause 151, any other suitable clause, or any combination of suitable clauses, wherein the blood monocyte cell is a purified blood monocyte cell.

153. The method of clause 145, any other suitable clause, or any combination of suitable clauses, wherein the somatic cell consists of a blood monocyte cell.

154. The method of clause 153, any other suitable clause, or any combination of suitable clauses, wherein the blood monocyte cell is a purified blood monocyte cell.

155. The method of clause 145, any other suitable clause, or any combination of suitable clauses, wherein the somatic cell comprises a muscle cell, a blood cell, a skin cell, a nerve cell, or any combination thereof.

156. The method of clause 145, any other suitable clause, or any combination of suitable clauses, wherein the somatic cell comprises an immune cell.

157. The method of clause 145, any other suitable clause, or any combination of suitable clauses, wherein the somatic cell comprises a muscle cell.

158. The method of clause 145, any other suitable clause, or any combination of suitable clauses, wherein the somatic cell comprises a blood cell.

159. The method of clause 145, any other suitable clause, or any combination of suitable clauses, wherein the somatic cell comprises a skin cell.

160. The method of clause 145, any other suitable clause, or any combination of suitable clauses, wherein the somatic cell comprises a nerve cell.

161. The method of clause 145, any other suitable clause, or any combination of suitable clauses, wherein the plurality of determined DMRs are selected from the DMRs in Table 1, the DMRs in Table 2, the DMRs in Table 3, the DMRs in Table 4, and any combination thereof.

162. The method of clause 145, any other suitable clause, or any combination of suitable clauses, wherein the plurality of determined DMRs are selected from the DMRs in Table 1.

163. The method of clause 145, any other suitable clause, or any combination of suitable clauses, wherein the plurality of determined DMRs are selected from the DMRs in Table 2.

164. The method of clause 145, any other suitable clause, or any combination of suitable clauses, wherein the plurality of determined DMRs are selected from the DMRs in Table 3.

165. The method of clause 145, any other suitable clause, or any combination of suitable clauses, wherein the plurality of determined DMRs are selected from the DMRs in Table 4.

166. The method of clause 145, any other suitable clause, or any combination of suitable clauses, wherein the plurality of distinct DMRs comprises at least 10 distinct DMRs.

167. The method of clause 145, any other suitable clause, or any combination of suitable clauses, wherein the plurality of distinct DMRs comprises 2 to about 10 distinct DMRs.

168. The method of clause 145, any other suitable clause, or any combination of suitable clauses, wherein the plurality of distinct DMRs comprises 5 to about 15 distinct DMRs.

169. The method of clause 145, any other suitable clause, or any combination of suitable clauses, wherein the plurality of distinct DMRs comprises 3 to about 8 distinct DMRs.

170. The method of clause 145, any other suitable clause, or any combination of suitable clauses, wherein the plurality of distinct DMRs comprises 4 to about 12 distinct DMRs.

171. The method of clause 145, any other suitable clause, or any combination of suitable clauses, wherein the plurality of distinct DMRs comprises about 20 to about 30 distinct DMRs.

172. The method of clause 145, any other suitable clause, or any combination of suitable clauses, wherein the plurality of distinct DMRs comprises about 10 to about 1000 distinct DMRs.

173. The method of clause 145, any other suitable clause, or any combination of suitable clauses, wherein the plurality of distinct DMRs comprises about 10 to about 15 distinct DMRs.

174. The method of clause 145, any other suitable clause, or any combination of suitable clauses, wherein the plurality of distinct DMRs comprises about 10 to about 50 distinct DMRs.

175. The method of clause 145, any other suitable clause, or any combination of suitable clauses, wherein the plurality of distinct DMRs comprises about 10 to about 100 distinct DMRs.

176. The method of clause 145, any other suitable clause, or any combination of suitable clauses, wherein the plurality of determined DMRs comprises about 50 to about 100 DMRs.

177. The method of clause 145, any other suitable clause, or any combination of suitable clauses, wherein the plurality of determined DMRs comprises about 100 to about 150 DMRs.

178. The method of clause 145, any other suitable clause, or any combination of suitable clauses, wherein the plurality of determined DMRs comprises about 150 to about 200 DMRs.

179. The method of clause 145, any other suitable clause, or any combination of suitable clauses, wherein the plurality of determined DMRs comprises about 200 to about 250 DMRs.

180. The method of clause 145, any other suitable clause, or any combination of suitable clauses, wherein the plurality of determined DMRs comprises about 250 to about 300 DMRs.

181. The method of clause 145, any other suitable clause, or any combination of suitable clauses, wherein the plurality of determined DMRs comprises about 300 to about 350 DMRs.

182. The method of clause 145, any other suitable clause, or any combination of suitable clauses, wherein the plurality of determined DMRs comprises about 350 to about 400 DMRs.

183. The method of clause 145, any other suitable clause, or any combination of suitable clauses, wherein the plurality of determined DMRs comprises about 400 to about 450 DMRs.

184. The method of clause 145, any other suitable clause, or any combination of suitable clauses, wherein the plurality of determined DMRs comprises about 450 to about 500 DMRs.

185. The method of clause 145, any other suitable clause, or any combination of suitable clauses, wherein the plurality of determined DMRs comprises about 500 to about 550 DMRs.

186. The method of clause 145, any other suitable clause, or any combination of suitable clauses, wherein the plurality of determined DMRs comprises about 550 to about 600 DMRs.

187. The method of clause 145, any other suitable clause, or any combination of suitable clauses, wherein the plurality of determined DMRs comprises about 600 to about 650 DMRs.

188. The method of clause 145, any other suitable clause, or any combination of suitable clauses, wherein the plurality of determined DMRs comprises about 650 to about 700 DMRs.

189. The method of clause 145, any other suitable clause, or any combination of suitable clauses, wherein the plurality of determined DMRs comprises about 700 to about 750 DMRs.

190. The method of clause 145, any other suitable clause, or any combination of suitable clauses, wherein the plurality of determined DMRs comprises about 750 to about 800 DMRs.

191. The method of clause 145, any other suitable clause, or any combination of suitable clauses, wherein the plurality of determined DMRs comprises about 800 to about 850 DMRs.

192. The method of clause 145, any other suitable clause, or any combination of suitable clauses, wherein the plurality of determined DMRs comprises about 850 to about 900 DMRs.

193. The method of clause 145, any other suitable clause, or any combination of suitable clauses, wherein the plurality of determined DMRs comprises about 900 to about 950 DMRs.

194. The method of clause 145, any other suitable clause, or any combination of suitable clauses, wherein the plurality of determined DMRs comprises about 950 to about 1000 DMRs.

195. The method of clause 145, any other suitable clause, or any combination of suitable clauses, wherein the plurality of distinct DMRs demonstrate an increase in DNA methylation.

196. The method of clause 145, any other suitable clause, or any combination of suitable clauses, wherein the plurality of distinct DMRs demonstrate a decrease in DNA methylation.

197. The method of clause 145, any other suitable clause, or any combination of suitable clauses, wherein the plurality of distinct DMRs are of a DMR associated gene category.

198. The method of clause 197, any other suitable clause, or any combination of suitable clauses, wherein the DMR associated gene category is a signaling gene category.

199. The method of clause 197, any other suitable clause, or any combination of suitable clauses, wherein the DMR associated gene category is a transcription gene category.

200. The method of clause 197, any other suitable clause, or any combination of suitable clauses, wherein the DMR associated gene category is a metabolism gene category.

201. The method of clause 197, any other suitable clause, or any combination of suitable clauses, wherein the DMR associated gene category is a receptor gene category.

202. The method of clause 197, any other suitable clause, or any combination of suitable clauses, wherein the DMR associated gene category is a cytoskeleton gene category.

203. The method of clause 197, any other suitable clause, or any combination of suitable clauses, wherein the DMR associated gene category is a development gene category.

204. The method of clause 197, any other suitable clause, or any combination of suitable clauses, wherein the DMR associated gene category is a transport gene category.

205. The method of clause 197, any other suitable clause, or any combination of suitable clauses, wherein the DMR associated gene category is an extracellular matrix gene category.

206. The method of clause 197, any other suitable clause, or any combination of suitable clauses, wherein the DMR associated gene category is an epigenetic gene category.

207. The method of clause 197, any other suitable clause, or any combination of suitable clauses, wherein the DMR associated gene category is a protease gene category.

208. The method of clause 197, any other suitable clause, or any combination of suitable clauses, wherein the DMR associated gene category is a proteolysis gene category.

209. The method of clause 197, any other suitable clause, or any combination of suitable clauses, wherein the DMR associated gene category is a translation gene category.

210. The method of clause 197, any other suitable clause, or any combination of suitable clauses, wherein the DMR associated gene category is a binding protein gene category.

211. The method of clause 197, any other suitable clause, or any combination of suitable clauses, wherein the DMR associated gene category is a growth factor/cytokine gene category.

212. The method of clause 197, any other suitable clause, or any combination of suitable clauses, wherein the DMR associated gene category is a cell cycle gene category.

213. The method of clause 197, any other suitable clause, or any combination of suitable clauses, wherein the DMR associated gene category is an immune gene category.

214. The method of clause 197, any other suitable clause, or any combination of suitable clauses, wherein the DMR associated gene category is an apoptosis gene category.

215. The method of clause 197, any other suitable clause, or any combination of suitable clauses, wherein the DMR associated gene category is a Golgi gene category.

216. The method of clause 197, any other suitable clause, or any combination of suitable clauses, wherein the DMR associated gene category is an EST gene category.

217. The method of clause 197, any other suitable clause, or any combination of suitable clauses, wherein the DMR associated gene category is an electron transport gene category.

218. The method of clause 197, any other suitable clause, or any combination of suitable clauses, wherein the DMR associated gene category is a protein binding gene category.

219. The method of clause 145, any other suitable clause, or any combination of suitable clauses, wherein the DMR associated gene category is a DNA repair gene category.

220. The method of clause 145, any other suitable clause, or any combination of suitable clauses, wherein the human subject is a male subject.

221. The method of clause 145, any other suitable clause, or any combination of suitable clauses, wherein the human subject is a female subject.

222. The method of clause 145, any other suitable clause, or any combination of suitable clauses, wherein the human subject is a Caucasian subject.

223. The method of clause 145, any other suitable clause, or any combination of suitable clauses, wherein the human subject is an African-American subject.

224. The method of clause 145, any other suitable clause, or any combination of suitable clauses, whether the determination of whether the human subject has or is at increased risk of having rheumatoid arthritis further comprises determination of smoking status of the human subject, nutritional status of the human subject, alcohol consumption of the human subject, or any combination thereof.

225. The method of clause 145, any other suitable clause, or any combination of suitable clauses, wherein the rheumatoid arthritis comprises a rheumatoid factor positive rheumatoid arthritis.

226. The method of clause 145, any other suitable clause, or any combination of suitable clauses, wherein the rheumatoid arthritis comprises a rheumatoid factor negative rheumatoid arthritis.

227. The method of clause 145, any other suitable clause, or any combination of suitable clauses, wherein the rheumatoid arthritis comprises juvenile rheumatoid arthritis.

228. The method of clause 145, any other suitable clause, or any combination of suitable clauses, wherein the rheumatoid arthritis comprises juvenile idiopathic arthritis.

229. The method of clause 145, any other suitable clause, or any combination of suitable clauses, further comprising treating the human subject.

230. The method of clause 229, any other suitable clause, or any combination of suitable clauses, wherein the treating comprises administering a medication for treatment of rheumatoid arthritis.

231. The method of clause 230, any other suitable clause, or any combination of suitable clauses, wherein the medication is an antibody.

232. The method of clause 230, any other suitable clause, or any combination of suitable clauses, wherein the medication is an anti-inflammatory.

233. The method of clause 232, any other suitable clause, or any combination of suitable clauses, wherein the anti-inflammatory is a steroid.

234. The method of clause 233, any other suitable clause, or any combination of suitable clauses, wherein the steroid comprises a corticosteroid or a salt thereof.

235. The method of clause 232, any other suitable clause, or any combination of suitable clauses, wherein the anti-inflammatory is a non-steroidal anti-inflammatory drug (NSAID).

236. The method of clause 235, any other suitable clause, or any combination of suitable clauses, wherein the NSAID comprises ibuprofen, naproxen sodium, or a salt thereof.

237. The method of clause 230, any other suitable clause, or any combination of suitable clauses, wherein the medication comprises methotrexate or a salt thereof.

238. The method of clause 230, any other suitable clause, or any combination of suitable clauses, wherein the medication comprises leflunomide or a salt thereof.

239. The method of clause 230, any other suitable clause, or any combination of suitable clauses, wherein the medication comprises hydroxychloroquine or a salt thereof.

240. The method of clause 230, any other suitable clause, or any combination of suitable clauses, wherein the medication comprises sulfasalazine or a salt thereof.

241. The method of clause 230, any other suitable clause, or any combination of suitable clauses, wherein the medication comprises abatacept.

242. The method of clause 230, any other suitable clause, or any combination of suitable clauses, wherein the medication comprises adalimumab.

243. The method of clause 230, any other suitable clause, or any combination of suitable clauses, wherein the medication comprises anakinra.

244. The method of clause 230, any other suitable clause, or any combination of suitable clauses, wherein the medication comprises certolizumab.

245. The method of clause 230, any other suitable clause, or any combination of suitable clauses, wherein the medication comprises etanercept.

246. The method of clause 230, any other suitable clause, or any combination of suitable clauses, wherein the medication comprises golimumab.

247. The method of clause 230, any other suitable clause, or any combination of suitable clauses, wherein the medication comprises infliximab.

248. The method of clause 230, any other suitable clause, or any combination of suitable clauses, wherein the medication comprises rituximab.

249. The method of clause 230, any other suitable clause, or any combination of suitable clauses, wherein the medication comprises sarilumab.

250. The method of clause 230, any other suitable clause, or any combination of suitable clauses, wherein the medication comprises tocilizumab.

251. The method of clause 145, any other suitable clause, or any combination of suitable clauses, further comprising transmitting data, a result, or both, via an electronic communication medium.

252. A kit comprising at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct primers or pairs of primers, each distinct primer or pairs of primers comprising a distinct sequence complementary to a distinct DMR sequence selected from the DMRs in Table 1, the DMRs in Table 2, the DMRs in Table 3, the DMRs in Table 4, and any combination thereof; and a container.

253. The kit of clause 252, any other suitable clause, or any combination of suitable clauses, wherein the distinct primers or pairs of primers each further comprise a unique barcode.

254. The kit of clause 252, any other suitable clause, or any combination of suitable clauses, wherein the distinct primers or pairs of primers are not bound to an array or a microarray.

255. The kit of clause 252, any other suitable clause, or any combination of suitable clauses, wherein the distinct primers or pairs of primers are bound to an array or a microarray.

256. The kit of clause 252, any other suitable clause, or any combination of suitable clauses, wherein the distinct primers or pairs of primers comprise DNA.

257. The kit of clause 252, any other suitable clause, or any combination of suitable clauses, wherein the distinct DMR sequences are selected from the DMRs in Table 1.

258. The kit of clause 252, any other suitable clause, or any combination of suitable clauses, wherein the distinct DMR sequences are selected from the DMRs in Table 2.

259. The kit of clause 252, any other suitable clause, or any combination of suitable clauses, wherein the distinct DMR sequences are selected from the DMRs in Table 3.

260. The kit of clause 252, any other suitable clause, or any combination of suitable clauses, wherein the distinct DMR sequences are selected from the DMRs in Table 4.

261. The kit of clause 252, any other suitable clause, or any combination of suitable clauses, wherein the distinct DMR sequences comprises about 50 to about 100 DMRs.

262. The kit of clause 252, any other suitable clause, or any combination of suitable clauses, wherein the distinct DMR sequences comprises about 100 to about 150 DMRs.

263. The kit of clause 252, any other suitable clause, or any combination of suitable clauses, wherein the distinct DMR sequences comprises about 150 to about 200 DMRs.

264. The kit of clause 252, any other suitable clause, or any combination of suitable clauses, wherein the distinct DMR sequences comprises about 200 to about 250 DMRs.

265. The kit of clause 252, any other suitable clause, or any combination of suitable clauses, wherein the distinct DMR sequences comprises about 250 to about 300 DMRs.

266. The kit of clause 252, any other suitable clause, or any combination of suitable clauses, wherein the distinct DMR sequences comprises about 300 to about 350 DMRs.

267. The kit of clause 252, any other suitable clause, or any combination of suitable clauses, wherein the distinct DMR sequences comprises about 350 to about 400 DMRs.

268. The kit of clause 252, any other suitable clause, or any combination of suitable clauses, wherein the distinct DMR sequences comprises about 400 to about 450 DMRs.

269. The kit of clause 252, any other suitable clause, or any combination of suitable clauses, wherein the distinct DMR sequences comprises about 450 to about 500 DMRs.

270. The kit of clause 252, any other suitable clause, or any combination of suitable clauses, wherein the distinct DMR sequences comprises about 500 to about 550 DMRs.

271. The kit of clause 252, any other suitable clause, or any combination of suitable clauses, wherein the distinct DMR sequences comprises about 550 to about 600 DMRs.

272. The kit of clause 252, any other suitable clause, or any combination of suitable clauses, wherein the distinct DMR sequences comprises about 600 to about 650 DMRs.

273. The kit of clause 252, any other suitable clause, or any combination of suitable clauses, wherein the distinct DMR sequences comprises about 650 to about 700 DMRs.

274. The kit of clause 252, any other suitable clause, or any combination of suitable clauses, wherein the distinct DMR sequences comprises about 700 to about 750 DMRs.

275. The kit of clause 252, any other suitable clause, or any combination of suitable clauses, wherein the distinct DMR sequences comprises about 750 to about 800 DMRs.

276. The kit of clause 252, any other suitable clause, or any combination of suitable clauses, wherein the distinct DMR sequences comprises about 800 to about 850 DMRs.

277. The kit of clause 252, any other suitable clause, or any combination of suitable clauses, wherein the distinct DMR sequences comprises about 850 to about 900 DMRs.

278. The kit of clause 252, any other suitable clause, or any combination of suitable clauses, wherein the distinct DMR sequences comprises about 900 to about 950 DMRs.

279. The kit of clause 252, any other suitable clause, or any combination of suitable clauses, wherein the distinct DMR sequences comprises about 950 to about 1000 DMRs.

280. The kit of clause 252, any other suitable clause, or any combination of suitable clauses, wherein the distinct DMR sequences are of a DMR associated gene category.

281. The kit of clause 280, any other suitable clause, or any combination of suitable clauses, wherein the DMR associated gene category is a signaling gene category.

282. The kit of clause 280, any other suitable clause, or any combination of suitable clauses, wherein the DMR associated gene category is a transcription gene category.

283. The kit of clause 280, any other suitable clause, or any combination of suitable clauses, wherein the DMR associated gene category is a metabolism gene category.

284. The kit of clause 280, any other suitable clause, or any combination of suitable clauses, wherein the DMR associated gene category is a receptor gene category.

285. The kit of clause 280, any other suitable clause, or any combination of suitable clauses, wherein the DMR associated gene category is a cytoskeleton gene category.

286. The kit of clause 280, any other suitable clause, or any combination of suitable clauses, wherein the DMR associated gene category is a development gene category.

287. The kit of clause 280, any other suitable clause, or any combination of suitable clauses, wherein the DMR associated gene category is a transport gene category.

288. The kit of clause 280, any other suitable clause, or any combination of suitable clauses, wherein the DMR associated gene category is an extracellular matrix gene category.

289. The kit of clause 280, any other suitable clause, or any combination of suitable clauses, wherein the DMR associated gene category is an epigenetic gene category.

290. The kit of clause 280, any other suitable clause, or any combination of suitable clauses, wherein the DMR associated gene category is a protease gene category.

291. The kit of clause 280, any other suitable clause, or any combination of suitable clauses, wherein the DMR associated gene category is a proteolysis gene category.

292. The kit of clause 280, any other suitable clause, or any combination of suitable clauses, wherein the DMR associated gene category is a translation gene category.

293. The kit of clause 280, any other suitable clause, or any combination of suitable clauses, wherein the DMR associated gene category is a binding protein gene category.

294. The kit of clause 280, any other suitable clause, or any combination of suitable clauses, wherein the DMR associated gene category is a growth factor/cytokine gene category.

295. The kit of clause 280, any other suitable clause, or any combination of suitable clauses, wherein the DMR associated gene category is a cell cycle gene category.

296. The kit of clause 280, any other suitable clause, or any combination of suitable clauses, wherein the DMR associated gene category is an immune gene category.

297. The kit of clause 280, any other suitable clause, or any combination of suitable clauses, wherein the DMR associated gene category is an apoptosis gene category.

298. The kit of clause 280, any other suitable clause, or any combination of suitable clauses, wherein the DMR associated gene category is a Golgi gene category.

299. The kit of clause 280, any other suitable clause, or any combination of suitable clauses, wherein the DMR associated gene category is an EST gene category.

300. The kit of clause 280, any other suitable clause, or any combination of suitable clauses, wherein the DMR associated gene category is an electron transport gene category.

301. The kit of clause 280, any other suitable clause, or any combination of suitable clauses, wherein the DMR associated gene category is a protein binding gene category.

302. The kit of clause 252, any other suitable clause, or any combination of suitable clauses, wherein the DMR associated gene category is a DNA repair gene category.

303. A kit comprising at least about 1, 2, 3, 4, 5, 6, 7, 8, 9 10, 11, 12, 13 14, 14, 16, 17, 18, 19, 20, 30, 40, 50, 60, 70, 80, 90, 100, 200, 300, 400, 500, 600, 700, 800, 900, or 1000 distinct probes, each distinct probe complementary to a distinct DMR sequence selected from the DMRs in Table 1, the DMRs in Table 2, the DMRs in Table 3, the DMRs in Table 4, and any combination thereof; and a container.

304. The kit of clause 303, any other suitable clause, or any combination of suitable clauses, wherein the distinct probes further comprises at least one of fluorophore, a chromophore, a barcode, or any combination thereof.

305. The kit of clause 304, any other suitable clause, or any combination of suitable clauses, wherein each probe comprises a unique fluorophore, a chromophore, barcode, or any combination thereof.

306. The kit of clause 303, any other suitable clause, or any combination of suitable clauses, wherein the distinct primers or pairs of primers are not bound to an array or a microarray.

307. The kit of clause 303, any other suitable clause, or any combination of suitable clauses, wherein the distinct primers or pairs of primers are bound to an array or a microarray.

308. The kit of clause 303, any other suitable clause, or any combination of suitable clauses, wherein the distinct primers or pairs of primers comprise DNA.

309. The kit of clause 303, any other suitable clause, or any combination of suitable clauses, wherein the distinct DMR sequences are selected from the DMRs in Table 1.

310. The kit of clause 303, any other suitable clause, or any combination of suitable clauses, wherein the distinct DMR sequences are selected from the DMRs in Table 2.

311. The kit of clause 303, any other suitable clause, or any combination of suitable clauses, wherein the distinct DMR sequences are selected from the DMRs in Table 3.

312. The kit of clause 303, any other suitable clause, or any combination of suitable clauses, wherein the distinct DMR sequences are selected from the DMRs in Table 4.

313. The kit of clause 303, any other suitable clause, or any combination of suitable clauses, wherein the distinct DMR sequences comprises about 50 to about 100 DMRs.

314. The kit of clause 303, any other suitable clause, or any combination of suitable clauses, wherein the distinct DMR sequences comprises about 100 to about 150 DMRs.

315. The kit of clause 303, any other suitable clause, or any combination of suitable clauses, wherein the distinct DMR sequences comprises about 150 to about 200 DMRs.

316. The kit of clause 303, any other suitable clause, or any combination of suitable clauses, wherein the distinct DMR sequences comprises about 200 to about 250 DMRs.

317. The kit of clause 303, any other suitable clause, or any combination of suitable clauses, wherein the distinct DMR sequences comprises about 250 to about 300 DMRs.

318. The kit of clause 303, any other suitable clause, or any combination of suitable clauses, wherein the distinct DMR sequences comprises about 300 to about 350 DMRs.

319. The kit of clause 303, any other suitable clause, or any combination of suitable clauses, wherein the distinct DMR sequences comprises about 350 to about 400 DMRs.

320. The kit of clause 303, any other suitable clause, or any combination of suitable clauses, wherein the distinct DMR sequences comprises about 400 to about 450 DMRs.

321. The kit of clause 303, any other suitable clause, or any combination of suitable clauses, wherein the distinct DMR sequences comprises about 450 to about 500 DMRs.

322. The kit of clause 303, any other suitable clause, or any combination of suitable clauses, wherein the distinct DMR sequences comprises about 500 to about 550 DMRs.

323. The kit of clause 303, any other suitable clause, or any combination of suitable clauses, wherein the distinct DMR sequences comprises about 550 to about 600 DMRs.

324. The kit of clause 303, any other suitable clause, or any combination of suitable clauses, wherein the distinct DMR sequences comprises about 600 to about 650 DMRs.

325. The kit of clause 303, any other suitable clause, or any combination of suitable clauses, wherein the distinct DMR sequences comprises about 650 to about 700 DMRs.

326. The kit of clause 303, any other suitable clause, or any combination of suitable clauses, wherein the distinct DMR sequences comprises about 700 to about 750 DMRs.

327. The kit of clause 303, any other suitable clause, or any combination of suitable clauses, wherein the distinct DMR sequences comprises about 750 to about 800 DMRs.

328. The kit of clause 303, any other suitable clause, or any combination of suitable clauses, wherein the distinct DMR sequences comprises about 800 to about 850 DMRs.

329. The kit of clause 303, any other suitable clause, or any combination of suitable clauses, wherein the distinct DMR sequences comprises about 850 to about 900 DMRs.

330. The kit of clause 303, any other suitable clause, or any combination of suitable clauses, wherein the distinct DMR sequences comprises about 900 to about 950 DMRs.

331. The kit of clause 303, any other suitable clause, or any combination of suitable clauses, wherein the distinct DMR sequences comprises about 950 to about 1000 DMRs.

332. The kit of clause 303, any other suitable clause, or any combination of suitable clauses, wherein the distinct DMR sequences are of a DMR associated gene category.

333. The kit of clause 332, any other suitable clause, or any combination of suitable clauses, wherein the DMR associated gene category is a signaling gene category.

334. The kit of clause 332, any other suitable clause, or any combination of suitable clauses, wherein the DMR associated gene category is a transcription gene category.

335. The kit of clause 332, any other suitable clause, or any combination of suitable clauses, wherein the DMR associated gene category is a metabolism gene category.

336. The kit of clause 332, any other suitable clause, or any combination of suitable clauses, wherein the DMR associated gene category is a receptor gene category.

337. The kit of clause 332, any other suitable clause, or any combination of suitable clauses, wherein the DMR associated gene category is a cytoskeleton gene category.

338. The kit of clause 332, any other suitable clause, or any combination of suitable clauses, wherein the DMR associated gene category is a development gene category.

339. The kit of clause 332, any other suitable clause, or any combination of suitable clauses, wherein the DMR associated gene category is a transport gene category.

340. The kit of clause 332, any other suitable clause, or any combination of suitable clauses, wherein the DMR associated gene category is an extracellular matrix gene category.

341. The kit of clause 332, any other suitable clause, or any combination of suitable clauses, wherein the DMR associated gene category is an epigenetic gene category.

342. The kit of clause 332, any other suitable clause, or any combination of suitable clauses, wherein the DMR associated gene category is a protease gene category.

343. The kit of clause 332, any other suitable clause, or any combination of suitable clauses, wherein the DMR associated gene category is a proteolysis gene category.

344. The kit of clause 332, any other suitable clause, or any combination of suitable clauses, wherein the DMR associated gene category is a translation gene category.

345. The kit of clause 332, any other suitable clause, or any combination of suitable clauses, wherein the DMR associated gene category is a binding protein gene category.

346. The kit of clause 332, any other suitable clause, or any combination of suitable clauses, wherein the DMR associated gene category is a growth factor/cytokine gene category.

347. The kit of clause 332, any other suitable clause, or any combination of suitable clauses, wherein the DMR associated gene category is a cell cycle gene category.

348. The kit of clause 332, any other suitable clause, or any combination of suitable clauses, wherein the DMR associated gene category is an immune gene category.

349. The kit of clause 332, any other suitable clause, or any combination of suitable clauses, wherein the DMR associated gene category is an apoptosis gene category.

350. The kit of clause 332, any other suitable clause, or any combination of suitable clauses, wherein the DMR associated gene category is a Golgi gene category.

351. The kit of clause 332, any other suitable clause, or any combination of suitable clauses, wherein the DMR associated gene category is an EST gene category.

352. The kit of clause 332, any other suitable clause, or any combination of suitable clauses, wherein the DMR associated gene category is an electron transport gene category.

353. The kit of clause 332, any other suitable clause, or any combination of suitable clauses, wherein the DMR associated gene category is a protein binding gene category.

354. The kit of clause 332, any other suitable clause, or any combination of suitable clauses, wherein the DMR associated gene category is a DNA repair gene category.

355. A method comprising:
obtaining a sample of a somatic cell from a human subject;
isolating deoxyribonucleic acid (DNA) from the sample, thereby obtaining isolated DNA;
fragmenting the DNA;
isolating fragmented methylated DNA;
detecting and determining a methylation level of a differential DNA methylation region (DMR) comprised in the isolated fragmented methylated DNA; and
comparing the methylation level of the DMR to a reference methylation level of a corresponding reference DMR;
wherein the comparing comprises comparing employing a computer comprising a computer processor and computer readable memory comprising computer readable instructions contained thereon,
wherein the detecting and determining comprises i) amplifying the isolated fragmented methylated DNA, ii) sequencing the isolated fragmented methylated DNA, an amplicon thereof, or both, or iii) employing an array, or iv) any combination thereof,
wherein a plurality of distinct DMRs are detected and compared, wherein the plurality comprises at least 10 distinct DMRs.

356. The method of clause 355, any other suitable clause, or any combination of suitable clauses, wherein the fragmenting is performed via sonication.

357. The method of clause 355, any other suitable clause, or any combination of suitable clauses, wherein the isolating the fragmented methylated DNA comprises methylated DNA immunoprecipitation (MeDIP).

358. The method of clause 355, any other suitable clause, or any combination of suitable clauses, wherein the somatic cell comprises a buccal cell.

359. The method of clause 355, any other suitable clause, or any combination of suitable clauses, wherein the somatic cell consists essentially of a buccal cell.

360. The method of clause 355, any other suitable clause, or any combination of suitable clauses, wherein the somatic cell consists of a buccal cell.

361. The method of clause 355, any other suitable clause, or any combination of suitable clauses, wherein the somatic cell comprises a blood monocyte cell.

362. The method of clause 361, any other suitable clause, or any combination of suitable clauses, wherein the blood monocyte cell is a purified blood monocyte cell.

363. The method of clause 355, any other suitable clause, or any combination of suitable clauses, wherein the somatic cell consists essentially of a blood monocyte cell.

364. The method of clause 363, any other suitable clause, or any combination of suitable clauses, wherein the blood monocyte cell is a purified blood monocyte cell.

365. The method of clause 355, any other suitable clause, or any combination of suitable clauses, wherein the somatic cell consists of a blood monocyte cell.

366. The method of clause 365, any other suitable clause, or any combination of suitable clauses, wherein the blood monocyte cell is a purified blood monocyte cell.

367. The method of clause 355, any other suitable clause, or any combination of suitable clauses, wherein the somatic cell comprises a muscle cell, a blood cell, a skin cell, a nerve cell, or any combination thereof.

368. The method of clause 355, any other suitable clause, or any combination of suitable clauses, wherein the somatic cell comprises an immune cell.

369. The method of clause 355, any other suitable clause, or any combination of suitable clauses, wherein the somatic cell comprises a muscle cell.

370. The method of clause 355, any other suitable clause, or any combination of suitable clauses, wherein the somatic cell comprises a blood cell.

371. The method of clause 355, any other suitable clause, or any combination of suitable clauses, wherein the somatic cell comprises a skin cell.

372. The method of clause 355, any other suitable clause, or any combination of suitable clauses, wherein the somatic cell comprises a nerve cell.

373. The method of clause 355, any other suitable clause, or any combination of suitable clauses, wherein the plurality of distinct DMRs are selected from the DMRs in Table 1, the DMRs in Table 2, the DMRs in Table 3, the DMRs in Table 4, and any combination thereof.

374. The method of clause 355, any other suitable clause, or any combination of suitable clauses, wherein the plurality of distinct DMRs are selected from the DMRs in Table 1.

375. The method of clause 355, any other suitable clause, or any combination of suitable clauses, wherein the plurality of distinct DMRs are selected from the DMRs in Table 2.

376. The method of clause 355, any other suitable clause, or any combination of suitable clauses, wherein the plurality of distinct DMRs are selected from the DMRs in Table 3.

377. The method of clause 355, any other suitable clause, or any combination of suitable clauses, wherein the plurality of distinct DMRs are selected from the DMRs in Table 4.

378. The method of clause 355, any other suitable clause, or any combination of suitable clauses, wherein the plurality of distinct DMRs comprises at least 10 distinct DMRs.

379. The method of clause 355, any other suitable clause, or any combination of suitable clauses, wherein the plurality of distinct DMRs comprises 2 to about 10 distinct DMRs.

380. The method of clause 355, any other suitable clause, or any combination of suitable clauses, wherein the plurality of distinct DMRs comprises 5 to about 15 distinct DMRs.

381. The method of clause 355, any other suitable clause, or any combination of suitable clauses, wherein the plurality of distinct DMRs comprises 3 to about 8 distinct DMRs.

382. The method of clause 355, any other suitable clause, or any combination of suitable clauses, wherein the plurality of distinct DMRs comprises 4 to about 12 distinct DMRs.

383. The method of clause 355, any other suitable clause, or any combination of suitable clauses, wherein the plurality of distinct DMRs comprises about 20 to about 30 distinct DMRs.

384. The method of clause 355, any other suitable clause, or any combination of suitable clauses, wherein the plurality of distinct DMRs comprises about 10 to about 1000 distinct DMRs.

385. The method of clause 355, any other suitable clause, or any combination of suitable clauses, wherein the plurality of distinct DMRs comprises about 10 to about 15 distinct DMRs.

386. The method of clause 355, any other suitable clause, or any combination of suitable clauses, wherein the plurality of distinct DMRs comprises about 10 to about 50 distinct DMRs.

387. The method of clause 355, any other suitable clause, or any combination of suitable clauses, wherein the plurality of distinct DMRs comprises about 10 to about 100 distinct DMRs.

388. The method of clause 355, any other suitable clause, or any combination of suitable clauses, wherein the plurality of distinct DMRs comprises about 50 to about 100 distinct DMRs.

389. The method of clause 355, any other suitable clause, or any combination of suitable clauses, wherein the plurality of distinct DMRs comprises about 100 to about 150 distinct DMRs.

390. The method of clause 355, any other suitable clause, or any combination of suitable clauses, wherein the plurality of distinct DMRs comprises about 150 to about 200 distinct DMRs.

391. The method of clause 355, any other suitable clause, or any combination of suitable clauses, wherein the plurality of distinct DMRs comprises about 200 to about 250 distinct DMRs.

392. The method of clause 355, any other suitable clause, or any combination of suitable clauses, wherein the plurality of distinct DMRs comprises about 250 to about 300 distinct DMRs.

393. The method of clause 355, any other suitable clause, or any combination of suitable clauses, wherein the plurality of distinct DMRs comprises about 300 to about 350 distinct DMRs.

394. The method of clause 355, any other suitable clause, or any combination of suitable clauses, wherein the plurality of distinct DMRs comprises about 350 to about 400 distinct DMRs.

395. The method of clause 355, any other suitable clause, or any combination of suitable clauses, wherein the plurality of distinct DMRs comprises about 400 to about 450 distinct DMRs.

396. The method of clause 355, any other suitable clause, or any combination of suitable clauses, wherein the plurality of distinct DMRs comprises about 450 to about 500 distinct DMRs.

397. The method of clause 355, any other suitable clause, or any combination of suitable clauses, wherein the plurality of distinct DMRs comprises about 500 to about 550 distinct DMRs.

398. The method of clause 355, any other suitable clause, or any combination of suitable clauses, wherein the plurality of distinct DMRs comprises about 550 to about 600 distinct DMRs.

399. The method of clause 355, any other suitable clause, or any combination of suitable clauses, wherein the plurality of distinct DMRs comprises about 600 to about 650 distinct DMRs.

400. The method of clause 355, any other suitable clause, or any combination of suitable clauses, wherein the plurality of distinct DMRs comprises about 650 to about 700 distinct DMRs.

401. The method of clause 355, any other suitable clause, or any combination of suitable clauses, wherein the plurality of distinct DMRs comprises about 700 to about 750 distinct DMRs.

402. The method of clause 355, any other suitable clause, or any combination of suitable clauses, wherein the plurality of distinct DMRs comprises about 750 to about 800 distinct DMRs.

403. The method of clause 355, any other suitable clause, or any combination of suitable clauses, wherein the plurality of distinct DMRs comprises about 800 to about 850 distinct DMRs.

404. The method of clause 355, any other suitable clause, or any combination of suitable clauses, wherein the plurality of distinct DMRs comprises about 850 to about 900 distinct DMRs.

405. The method of clause 355, any other suitable clause, or any combination of suitable clauses, wherein the plurality of distinct DMRs comprises about 900 to about 950 distinct DMRs.

406. The method of clause 355, any other suitable clause, or any combination of suitable clauses, wherein the plurality of distinct DMRs comprises about 950 to about 1000 distinct DMRs.

407. The method of clause 355, any other suitable clause, or any combination of suitable clauses, wherein the plurality of distinct DMRs demonstrate an increase in DNA methylation.

408. The method of clause 355, any other suitable clause, or any combination of suitable clauses, wherein the plurality of distinct DMRs demonstrate a decrease in DNA methylation.

409. The method of clause 355, any other suitable clause, or any combination of suitable clauses, wherein the plurality of distinct DMRs are of a DMR associated gene category.

410. The method of clause 409, any other suitable clause, or any combination of suitable clauses, wherein the DMR associated gene category is a signaling gene category.

411. The method of clause 409, any other suitable clause, or any combination of suitable clauses, wherein the DMR associated gene category is a transcription gene category.

412. The method of clause 409, any other suitable clause, or any combination of suitable clauses, wherein the DMR associated gene category is a metabolism gene category.

413. The method of clause 409, any other suitable clause, or any combination of suitable clauses, wherein the DMR associated gene category is a receptor gene category.

414. The method of clause 409, any other suitable clause, or any combination of suitable clauses, wherein the DMR associated gene category is a cytoskeleton gene category.

415. The method of clause 409, any other suitable clause, or any combination of suitable clauses, wherein the DMR associated gene category is a development gene category.

416. The method of clause 409, any other suitable clause, or any combination of suitable clauses, wherein the DMR associated gene category is a transport gene category.

417. The method of clause 409, any other suitable clause, or any combination of suitable clauses, wherein the DMR associated gene category is an extracellular matrix gene category.

418. The method of clause 409, any other suitable clause, or any combination of suitable clauses, wherein the DMR associated gene category is an epigenetic gene category.

419. The method of clause 409, any other suitable clause, or any combination of suitable clauses, wherein the DMR associated gene category is a protease gene category.

420. The method of clause 409, any other suitable clause, or any combination of suitable clauses, wherein the DMR associated gene category is a proteolysis gene category.

421. The method of clause 409, any other suitable clause, or any combination of suitable clauses, wherein the DMR associated gene category is a translation gene category.

422. The method of clause 409, any other suitable clause, or any combination of suitable clauses, wherein the DMR associated gene category is a binding protein gene category.

423. The method of clause 409, any other suitable clause, or any combination of suitable clauses, wherein the DMR associated gene category is a growth factor/cytokine gene category.

424. The method of clause 409, any other suitable clause, or any combination of suitable clauses, wherein the DMR associated gene category is a cell cycle gene category.

425. The method of clause 409, any other suitable clause, or any combination of suitable clauses, wherein the DMR associated gene category is an immune gene category.

426. The method of clause 409, any other suitable clause, or any combination of suitable clauses, wherein the DMR associated gene category is an apoptosis gene category.

427. The method of clause 409, any other suitable clause, or any combination of suitable clauses, wherein the DMR associated gene category is a Golgi gene category.

428. The method of clause 409, any other suitable clause, or any combination of suitable clauses, wherein the DMR associated gene category is an EST gene category.

429. The method of clause 409, any other suitable clause, or any combination of suitable clauses, wherein the DMR associated gene category is an electron transport gene category.

430. The method of clause 409, any other suitable clause, or any combination of suitable clauses, wherein the DMR associated gene category is a protein binding gene category.

431. The method of clause 409, any other suitable clause, or any combination of suitable clauses, wherein the DMR associated gene category is a DNA repair gene category.

432. The method of clause 355, any other suitable clause, or any combination of suitable clauses, comprising sequencing, wherein the sequencing comprises sequencing by synthesis, ion semiconductor sequencing, single molecule real time sequencing, nanopore sequencing, next-generation sequencing, or any combination thereof.

433. The method of clause 355, any other suitable clause, or any combination of suitable clauses, wherein the detected DMRs comprise DMRs from at least about: 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about: 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.

434. The method of clause 355, any other suitable clause, or any combination of suitable clauses, wherein the DMRs that are determined and compared, individually, range from about 100 to about 17000 adjacent nucleotides.

435. The method of clause 355, any other suitable clause, or any combination of suitable clauses, wherein at least a plurality of the DMRs that are determined and compared comprise a CpG density of 1 to about 10 CpG per 100 nucleotides.

436. The method of clause 435, any other suitable clause, or any combination of suitable clauses, wherein at least a plurality of the DMRs that are determined and compared comprise a CpG density of less than about 10 CpG per 100 nucleotides.

437. The method of clause 435, any other suitable clause, or any combination of suitable clauses, wherein at least a plurality of the DMRs that are determined and compared comprise a CpG density of less than about 5 CpG per 100 nucleotides.

438. The method of clause 435, any other suitable clause, or any combination of suitable clauses, wherein at least a plurality of the DMRs that are determined and compared comprise a CpG density of less than about 3 CpG per 100 nucleotides.

439. The method of clause 435, any other suitable clause, or any combination of suitable clauses, wherein at least a plurality of the DMRs that are determined and compared comprise a CpG density of between 1 CpG and 3 CpG per 100 nucleotides.

440. The method of clause 355, any other suitable clause, or any combination of suitable clauses, wherein at least about 50, 60, or 70 percent of the DMRs that are determined and compared are hypermethylated when compared, individually, to individual reference methylation levels of corresponding individual reference DMRs.

441. The method of clause 355, any other suitable clause, or any combination of suitable clauses, wherein at least about 30, 40, or 50 percent of the DMRs that are determined and compared are hypomethylated when compared, individually, to individual reference methylation levels of corresponding individual reference DMRs.

442. The method of clause 355, any other suitable clause, or any combination of suitable clauses, wherein the human subject is a male subject.

443. The method of clause 355, any other suitable clause, or any combination of suitable clauses, wherein the human subject is a female subject.

444. The method of clause 355, any other suitable clause, or any combination of suitable clauses, wherein the human subject is a Caucasian subject.

445. The method of clause 355, any other suitable clause, or any combination of suitable clauses, wherein the human subject is an African-American subject.

446. The method of clause 355, any other suitable clause, or any combination of suitable clauses, wherein the method further comprises determining a risk of the human subject having a disease or condition, wherein the determining is performed with a computer comprising a computer processor and computer readable memory comprising computer readable instructions.

447. The method of clause 446, any other suitable clause, or any combination of suitable clauses, wherein the disease or condition is rheumatoid arthritis.

448. The method of clause 447, any other suitable clause, or any combination of suitable clauses, wherein the rheumatoid arthritis comprises a rheumatoid factor positive rheumatoid arthritis.

449. The method of clause 447, any other suitable clause, or any combination of suitable clauses, wherein the rheumatoid arthritis comprises a rheumatoid factor negative rheumatoid arthritis.

450. The method of clause 447, any other suitable clause, or any combination of suitable clauses, wherein the rheumatoid arthritis comprises juvenile rheumatoid arthritis.

451. The method of clause 447, any other suitable clause, or any combination of suitable clauses, wherein the rheumatoid arthritis comprises juvenile idiopathic arthritis.

452. The method of clause 446, any other suitable clause, or any combination of suitable clauses, wherein the risk of the human subject further comprises determination of smoking status of the human subject, nutritional status of the human subject, alcohol consumption of the human subject, or any combination thereof.

453. The method of clause 355, any other suitable clause, or any combination of suitable clauses, further comprising performing an additional analysis using a computer.

454. The method of clause 453, any other suitable clause, or any combination of suitable clauses, wherein the additional analysis comprises a principle component analysis (PCA), a dendrogram analysis, a machine learning analysis, or any combination thereof.

455. The method of clause 453, any other suitable clause, or any combination of suitable clauses, wherein the additional analysis generates data points, and wherein the data points in the additional analysis are grouped into two spatially distinct categories comprising a first category and a second category.

456. The method of clause 455, any other suitable clause, or any combination of suitable clauses, wherein the first category indicates the human subject is at increased risk of having a disease or condition and wherein the second category indicates the human subject is not at increased risk of having the disease or condition.

457. The method of clause 355, any other suitable clause, or any combination of suitable clauses, further comprising treating the human subject.

458. The method of clause 457, any other suitable clause, or any combination of suitable clauses, wherein the treating comprises administering a medication for treatment of rheumatoid arthritis.

459. The method of clause 458, any other suitable clause, or any combination of suitable clauses, wherein the medication is an antibody.

460. The method of clause 458, any other suitable clause, or any combination of suitable clauses, wherein the medication is an anti-inflammatory.

461. The method of clause 460, any other suitable clause, or any combination of suitable clauses, wherein the anti-inflammatory is a steroid.

462. The method of clause 461, any other suitable clause, or any combination of suitable clauses, wherein the steroid comprises a corticosteroid or a salt thereof.

463. The method of clause 460, any other suitable clause, or any combination of suitable clauses, wherein the anti-inflammatory is a non-steroidal anti-inflammatory drug (NSAID).

464. The method of clause 463, any other suitable clause, or any combination of suitable clauses, wherein the NSAID comprises ibuprofen, naproxen sodium, or a salt thereof.

465. The method of clause 458, any other suitable clause, or any combination of suitable clauses, wherein the medication comprises methotrexate or a salt thereof.

466. The method of clause 458, any other suitable clause, or any combination of suitable clauses, wherein the medication comprises leflunomide or a salt thereof.

467. The method of clause 458, any other suitable clause, or any combination of suitable clauses, wherein the medication comprises hydroxychloroquine or a salt thereof.

468. The method of clause 458, any other suitable clause, or any combination of suitable clauses, wherein the medication comprises sulfasalazine or a salt thereof.

469. The method of clause 458, any other suitable clause, or any combination of suitable clauses, wherein the medication comprises abatacept.

470. The method of clause 458, any other suitable clause, or any combination of suitable clauses, wherein the medication comprises adalimumab.

471. The method of clause 458, any other suitable clause, or any combination of suitable clauses, wherein the medication comprises anakinra.

472. The method of clause 458, any other suitable clause, or any combination of suitable clauses, wherein the medication comprises certolizumab.

473. The method of clause 458, any other suitable clause, or any combination of suitable clauses, wherein the medication comprises etanercept.

474. The method of clause 458, any other suitable clause, or any combination of suitable clauses, wherein the medication comprises golimumab.

475. The method of clause 458, any other suitable clause, or any combination of suitable clauses, wherein the medication comprises infliximab.

476. The method of clause 458, any other suitable clause, or any combination of suitable clauses, wherein the medication comprises rituximab.

477. The method of clause 458, any other suitable clause, or any combination of suitable clauses, wherein the medication comprises sarilumab.

478. The method of clause 458, any other suitable clause, or any combination of suitable clauses, wherein the medication comprises tocilizumab.

479. The method of clause 355, any other suitable clause, or any combination of suitable clauses, further comprising transmitting data, a result, or both, via an electronic communication medium.

480. A method comprising:
obtaining a sample of a somatic cell from a human subject;
isolating deoxyribonucleic acid (DNA) from the sample, thereby obtaining isolated DNA;
fragmenting the DNA;
isolating fragmented methylated DNA;
detecting and determining a methylation level of a differential DNA methylation region (DMR) comprised in the isolated fragmented methylated DNA; and
comparing the methylation level of the DMR to a reference methylation level of a corresponding reference DMR;
wherein the comparing comprises comparing employing a computer comprising a computer processor and computer readable memory comprising computer readable instructions contained thereon,
wherein the detecting and determining comprises i) amplifying the isolated fragmented methylated DNA, ii) sequencing the isolated fragmented methylated DNA, an amplicon thereof, or both, or iii) employing an array, or iv) any combination thereof; and
wherein a plurality of determined DMRs are sufficient to determine, from a process comprising the comparing and employing a computer, whether the human subject has or is at increased risk of having rheumatoid arthritis.

481. The method of clause 480, any other suitable clause, or any combination of suitable clauses, wherein the fragmenting is performed via sonication.

482. The method of clause 480, any other suitable clause, or any combination of suitable clauses, wherein the isolating the fragmented methylated DNA comprises methylated DNA immunoprecipitation (MeDIP).

483. The method of clause 480, any other suitable clause, or any combination of suitable clauses, wherein the somatic cell comprises a buccal cell.

484. The method of clause 480, any other suitable clause, or any combination of suitable clauses, wherein the somatic cell consists essentially of a buccal cell.

485. The method of clause 480, any other suitable clause, or any combination of suitable clauses, wherein the somatic cell consists of a buccal cell.

486. The method of clause 480, any other suitable clause, or any combination of suitable clauses, wherein the somatic cell comprises a blood monocyte cell.

487. The method of clause 486, any other suitable clause, or any combination of suitable clauses, wherein the blood monocyte cell is a purified blood monocyte cell.

488. The method of clause 480, any other suitable clause, or any combination of suitable clauses, wherein the somatic cell consists essentially of a blood monocyte cell.

489. The method of clause 488, any other suitable clause, or any combination of suitable clauses, wherein the blood monocyte cell is a purified blood monocyte cell.

490. The method of clause 480, any other suitable clause, or any combination of suitable clauses, wherein the somatic cell consists of a blood monocyte cell.

491. The method of clause 490, any other suitable clause, or any combination of suitable clauses, wherein the blood monocyte cell is a purified blood monocyte cell.

492. The method of clause 480, any other suitable clause, or any combination of suitable clauses, wherein the somatic cell comprises a muscle cell, a blood cell, a skin cell, a nerve cell, or any combination thereof.

493. The method of clause 480, any other suitable clause, or any combination of suitable clauses, wherein the somatic cell comprises an immune cell.

494. The method of clause 480, any other suitable clause, or any combination of suitable clauses, wherein the somatic cell comprises a muscle cell.

495. The method of clause 480, any other suitable clause, or any combination of suitable clauses, wherein the somatic cell comprises a blood cell.

496. The method of clause 480, any other suitable clause, or any combination of suitable clauses, wherein the somatic cell comprises a skin cell.

497. The method of clause 480, any other suitable clause, or any combination of suitable clauses, wherein the somatic cell comprises a nerve cell.

498. The method of clause 480, any other suitable clause, or any combination of suitable clauses, wherein the plurality of determined DMRs are selected from the DMRs in Table 1, the DMRs in Table 2, the DMRs in Table 3, the DMRs in Table 4, and any combination thereof.

499. The method of clause 480, any other suitable clause, or any combination of suitable clauses, wherein the plurality of determined DMRs are selected from the DMRs in Table 1.

500. The method of clause 480, any other suitable clause, or any combination of suitable clauses, wherein the plurality of determined DMRs are selected from the DMRs in Table 2.

501. The method of clause 480, any other suitable clause, or any combination of suitable clauses, wherein the plurality of determined DMRs are selected from the DMRs in Table 3.

502. The method of clause 480, any other suitable clause, or any combination of suitable clauses, wherein the plurality of determined DMRs are selected from the DMRs in Table 4.

503. The method of clause 480, any other suitable clause, or any combination of suitable clauses, wherein the plurality of determined DMRs comprises about 50 to about 100 DMRs.

504. The method of clause 480, any other suitable clause, or any combination of suitable clauses, wherein the plurality of determined DMRs comprises about 100 to about 150 DMRs.

505. The method of clause 480, any other suitable clause, or any combination of suitable clauses, wherein the plurality of determined DMRs comprises about 150 to about 200 DMRs.

506. The method of clause 480, any other suitable clause, or any combination of suitable clauses, wherein the plurality of determined DMRs comprises about 200 to about 250 DMRs.

507. The method of clause 480, any other suitable clause, or any combination of suitable clauses, wherein the plurality of determined DMRs comprises about 250 to about 300 DMRs.

508. The method of clause 480, any other suitable clause, or any combination of suitable clauses, wherein the plurality of determined DMRs comprises about 300 to about 350 DMRs.

509. The method of clause 480, any other suitable clause, or any combination of suitable clauses, wherein the plurality of determined DMRs comprises about 350 to about 400 DMRs.

510. The method of clause 480, any other suitable clause, or any combination of suitable clauses, wherein the plurality of determined DMRs comprises about 400 to about 450 DMRs.

511. The method of clause 480, any other suitable clause, or any combination of suitable clauses, wherein the plurality of determined DMRs comprises about 450 to about 500 DMRs.

512. The method of clause 480, any other suitable clause, or any combination of suitable clauses, wherein the plurality of determined DMRs comprises about 500 to about 550 DMRs.

513. The method of clause 480, any other suitable clause, or any combination of suitable clauses, wherein the plurality of determined DMRs comprises about 550 to about 600 DMRs.

514. The method of clause 480, any other suitable clause, or any combination of suitable clauses, wherein the plurality of determined DMRs comprises about 600 to about 650 DMRs.

515. The method of clause 480, any other suitable clause, or any combination of suitable clauses, wherein the plurality of determined DMRs comprises about 650 to about 700 DMRs.

516. The method of clause 480, any other suitable clause, or any combination of suitable clauses, wherein the plurality of determined DMRs comprises about 700 to about 750 DMRs.

517. The method of clause 480, any other suitable clause, or any combination of suitable clauses, wherein the plurality of determined DMRs comprises about 750 to about 800 DMRs.

518. The method of clause 480, any other suitable clause, or any combination of suitable clauses, wherein the plurality of determined DMRs comprises about 800 to about 850 DMRs.

519. The method of clause 480, any other suitable clause, or any combination of suitable clauses, wherein the plurality of determined DMRs comprises about 850 to about 900 DMRs.

520. The method of clause 480, any other suitable clause, or any combination of suitable clauses, wherein the plurality of determined DMRs comprises about 900 to about 950 DMRs.

521. The method of clause 480, any other suitable clause, or any combination of suitable clauses, wherein the plurality of determined DMRs comprises about 950 to about 1000 DMRs.

522. The method of clause 480, any other suitable clause, or any combination of suitable clauses, wherein the plurality of distinct DMRs demonstrate an increase in DNA methylation.

523. The method of clause 480, any other suitable clause, or any combination of suitable clauses, wherein the plurality of distinct DMRs demonstrate a decrease in DNA methylation.

524. The method of clause 480, any other suitable clause, or any combination of suitable clauses, wherein the plurality of distinct DMRs are of a DMR associated gene category.

525. The method of clause 524, any other suitable clause, or any combination of suitable clauses, wherein the DMR associated gene category is a signaling gene category.

526. The method of clause 524, any other suitable clause, or any combination of suitable clauses, wherein the DMR associated gene category is a transcription gene category.

527. The method of clause 524, any other suitable clause, or any combination of suitable clauses, wherein the DMR associated gene category is a metabolism gene category.

528. The method of clause 524, any other suitable clause, or any combination of suitable clauses, wherein the DMR associated gene category is a receptor gene category.

529. The method of clause 524, any other suitable clause, or any combination of suitable clauses, wherein the DMR associated gene category is a cytoskeleton gene category.

530. The method of clause 524, any other suitable clause, or any combination of suitable clauses, wherein the DMR associated gene category is a development gene category.

531. The method of clause 524, any other suitable clause, or any combination of suitable clauses, wherein the DMR associated gene category is a transport gene category.

532. The method of clause 524, any other suitable clause, or any combination of suitable clauses, wherein the DMR associated gene category is an extracellular matrix gene category.

533. The method of clause 524, any other suitable clause, or any combination of suitable clauses, wherein the DMR associated gene category is an epigenetic gene category.

534. The method of clause 524, any other suitable clause, or any combination of suitable clauses, wherein the DMR associated gene category is a protease gene category.

535. The method of clause 524, any other suitable clause, or any combination of suitable clauses, wherein the DMR associated gene category is a proteolysis gene category.

536. The method of clause 524, any other suitable clause, or any combination of suitable clauses, wherein the DMR associated gene category is a translation gene category.

537. The method of clause 524, any other suitable clause, or any combination of suitable clauses, wherein the DMR associated gene category is a binding protein gene category.

538. The method of clause 524, any other suitable clause, or any combination of suitable clauses, wherein the DMR associated gene category is a growth factor/cytokine gene category.

539. The method of clause 524, any other suitable clause, or any combination of suitable clauses, wherein the DMR associated gene category is a cell cycle gene category.

540. The method of clause 524, any other suitable clause, or any combination of suitable clauses, wherein the DMR associated gene category is an immune gene category.

541. The method of clause 524, any other suitable clause, or any combination of suitable clauses, wherein the DMR associated gene category is an apoptosis gene category.

542. The method of clause 524, any other suitable clause, or any combination of suitable clauses, wherein the DMR associated gene category is a Golgi gene category.

543. The method of clause 524, any other suitable clause, or any combination of suitable clauses, wherein the DMR associated gene category is an EST gene category.

544. The method of clause 524, any other suitable clause, or any combination of suitable clauses, wherein the DMR associated gene category is an electron transport gene category.

545. The method of clause 524, any other suitable clause, or any combination of suitable clauses, wherein the DMR associated gene category is a protein binding gene category.

546. The method of clause 480, any other suitable clause, or any combination of suitable clauses, wherein the DMR associated gene category is a DNA repair gene category.

547. The method of clause 480, any other suitable clause, or any combination of suitable clauses, wherein the human subject is a male subject.

548. The method of clause 480, any other suitable clause, or any combination of suitable clauses, wherein the human subject is a female subject.

549. The method of clause 480, any other suitable clause, or any combination of suitable clauses, wherein the human subject is a Caucasian subject.

550. The method of clause 480, any other suitable clause, or any combination of suitable clauses, wherein the human subject is an African-American subject.

551. The method of clause 480, any other suitable clause, or any combination of suitable clauses, whether the determination of whether the human subject has or is at increased risk of having rheumatoid arthritis further comprises determination of smoking status of the human subject, nutritional status of the human subject, alcohol consumption of the human subject, or any combination thereof.

552. The method of clause 480, any other suitable clause, or any combination of suitable clauses, wherein the rheumatoid arthritis comprises a rheumatoid factor positive rheumatoid arthritis.

553. The method of clause 480, any other suitable clause, or any combination of suitable clauses, wherein the rheumatoid arthritis comprises a rheumatoid factor negative rheumatoid arthritis.

554. The method of clause 480, any other suitable clause, or any combination of suitable clauses, wherein the rheumatoid arthritis comprises juvenile rheumatoid arthritis.

555. The method of clause 480, any other suitable clause, or any combination of suitable clauses, wherein the rheumatoid arthritis comprises juvenile idiopathic arthritis.

556. The method of clause 480, any other suitable clause, or any combination of suitable clauses, further comprising treating the human subject.

557. The method of clause 556, any other suitable clause, or any combination of suitable clauses, wherein the treating comprises administering a medication for treatment of rheumatoid arthritis.

558. The method of clause 557, any other suitable clause, or any combination of suitable clauses, wherein the medication is an antibody.

559. The method of clause 557, any other suitable clause, or any combination of suitable clauses, wherein the medication is an anti-inflammatory.

560. The method of clause 559, any other suitable clause, or any combination of suitable clauses, wherein the anti-inflammatory is a steroid.

561. The method of clause 560, any other suitable clause, or any combination of suitable clauses, wherein the steroid comprises a corticosteroid or a salt thereof.

562. The method of clause 559, any other suitable clause, or any combination of suitable clauses, wherein the anti-inflammatory is a non-steroidal anti-inflammatory drug (NSAID).

563. The method of clause 562, any other suitable clause, or any combination of suitable clauses, wherein the NSAID comprises ibuprofen, naproxen sodium, or a salt thereof.

564. The method of clause 557, any other suitable clause, or any combination of suitable clauses, wherein the medication comprises methotrexate or a salt thereof.

565. The method of clause 557, any other suitable clause, or any combination of suitable clauses, wherein the medication comprises leflunomide or a salt thereof.

566. The method of clause 557, any other suitable clause, or any combination of suitable clauses, wherein the medication comprises hydroxychloroquine or a salt thereof.

567. The method of clause 557, any other suitable clause, or any combination of suitable clauses, wherein the medication comprises sulfasalazine or a salt thereof.

568. The method of clause 557, any other suitable clause, or any combination of suitable clauses, wherein the medication comprises abatacept.

569. The method of clause 557, any other suitable clause, or any combination of suitable clauses, wherein the medication comprises adalimumab.

570. The method of clause 557, any other suitable clause, or any combination of suitable clauses, wherein the medication comprises anakinra.

571. The method of clause 557, any other suitable clause, or any combination of suitable clauses, wherein the medication comprises certolizumab.

572. The method of clause 557, any other suitable clause, or any combination of suitable clauses, wherein the medication comprises etanercept.

573. The method of clause 557, any other suitable clause, or any combination of suitable clauses, wherein the medication comprises golimumab.

574. The method of clause 557, any other suitable clause, or any combination of suitable clauses, wherein the medication comprises infliximab.

575. The method of clause 557, any other suitable clause, or any combination of suitable clauses, wherein the medication comprises rituximab.

576. The method of clause 557, any other suitable clause, or any combination of suitable clauses, wherein the medication comprises sarilumab.

577. The method of clause 557, any other suitable clause, or any combination of suitable clauses, wherein the medication comprises tocilizumab.

578. The method of clause 480, any other suitable clause, or any combination of suitable clauses, further comprising transmitting data, a result, or both, via an electronic communication medium.

### EXAMPLES

### Example 1

### Exemplary Sample Collection and Evaluation

In the instant example, exemplary samples were collected and analyzed. The methods of the instant example were utilized for data presentation in Examples 2-6.

### Clinical Sample Collection and Analysis

Two independent single centers (Arthritis Northwest, ANW, Spokane, WA, USA and Dx Biosamples, LLC in San Diego, California, USA) performed the prospective and open clinical study. The participant approval and informed consent was obtained from all participants prior to the clinical sample collection.

The arthritis severity was assessed for the Caucasian population with diagnostic rheumatoid factor autoantibody (RF) positive assay and cyclic citrullinated peptide autoantibody (CCP) positive assay. The arthritis severity was also assessed for both the Caucasian and African American populations with a more qualitative Clinical Disease Activity Index (CDAI) analysis with a high activity score of >22.1-76.0 and moderate activity score of 10.1-22.0. The CDAI was also supported with DAS28 and RAPID3 assays for the Caucasian population.

Three group comparisons (buccal from female with RA arthritis (case) or without (control)) were included for both Caucasian and African American populations. The females included Caucasians and African Americans with a mean group age of 54 years. Buccal samples were frozen and stored (−20° C.) for the subsequent epigenetic analysis. Blood was collected into BD Vacutainer CPT tubes (Becton, Dickinson and Company, Franklin Lakes, NJ) by ANW Spokane, WA and shipped overnight on ice for immediate processing. Monocytes were isolated using antibody-linked magnetic beads (Dynabeads CD14, Life Technologies #11149D).

### Cell Isolation and DNA Preparation

Human buccal samples were kept frozen and thawed for analysis. Blood samples were kept refrigerated before monocyte isolation. For monocyte isolation the blood was diluted 1:2 in Isolation Buffer (1×PBS, 0.1% BSA, 2 mM EDTA) then centrifuged at 600×g for 10 min at room temperature in a swinging bucket rotor. The plasma upper layer was discarded, and the blood resuspended to the original volume in Isolation Buffer. Magnetic beads (Dynabeads CD14, Life Technologies #11149D) were added to the blood samples (25 μL of pre-washed beads for 1 ml of blood sample). The mixture (beads-cells) was incubated for 20 min between 2° C. and 8° C. with gentle tilting and rotation. The mixture was then placed on a magnet for 2 minutes and the supernatant carefully removed. One ml of Isolation Buffer was added to the beads-cells mixture then homogenized and incubated 2 minutes on the magnet. This step was repeated at least 3 times. The bead-bound cells were resuspended in 100 μl 1×PBS pH 7.4 and genomic DNA isolation was performed.

Genomic DNA from buccal swabs or monocytes was prepared as follows: The buccal swabs were incubated with 750 μl of cell lysis solution (100 mM NaCl, 10 mM Tris, 25 mM EDTA, 0.5% SDS) and 3.5 μl of Proteinase K (20 mg/ml) for 3 hours in a heating block at 55° C. The lysis solution was transferred to a new tube and spun for 2 min at full speed. For the monocytes, 820 μl of DNA extraction buffer (50 mM Tris pH 8, 10 mM EDTA pH 8, 0.5% SDS) and 80 μl Proteinase K (20 mg/ml) were added to the bead-bound monocytes and the sample incubated at 55° C. for 2 hours under constant rotation. The samples were placed on a magnet where the magnetic beads were discarded and the supernatants containing the DNA kept. Then for both the buccal swabs and the monocytes, 300 μl of protein precipitation solution (Promega Genomic DNA Purification Kit, A795A, Madison, WI) was added, the sample was mixed and incubated on ice for 15 min, then spun at 4° C. at 13,500×g for 20 min. The supernatant was transferred to a fresh tube, then precipitated over night at −20° C. with 1 mL of cold 100% isopropanol and 2 μl glycoblue. The sample was then centrifuged at 4° C., 13,500×g for 20 min. The supernatant was discarded and the pellet washed with 70% cold ethanol then returned to freezer for 20-30 min. The sample was then centrifuged at 4° C., 13,500×g for 10 min. The supernatant was discarded, and the pellet air-dried for 5 min then resuspended in 100 μl nuclease free water. DNA concentration was measured using the Nanodrop (Thermo Fisher, Waltham, MA).

### Methylated DNA Immunoprecipitation (MeDIP)

Methylated DNA Immunoprecipitation (MeDIP) with genomic DNA was performed as follows: individual genomic DNA samples (2-4 ug of total DNA) were diluted to 130 μl with 1×Tris-EDTA (TE, 10 mM Tris, 1 mM EDTA) and sonicated with the Covaris M220 using the 300 bp setting. Fragment size was verified on a 2% E-gel agarose gel. The sonicated DNA was transferred from the Covaris tube to a 1.7 ml microfuge tube, and the volume was measured. The sonicated DNA was then diluted with TE buffer (10 mM Tris HCl, pH 7.5; 1 mM EDTA) to 400 μl, heat-denatured for 10 min at 95° C., then immediately cooled on ice for 10 min. Then 100 μl of 5×IP buffer and 5 μg of antibody (monoclonal mouse anti 5-methyl cytidine; Diagenode #C15200006) were added to the denatured sonicated DNA. The DNA-antibody mixture was incubated overnight on a rotator at 4° C. The following day magnetic beads (Dynabeads M-280 Sheep anti-Mouse IgG; 11201D) were pre-washed as follows: The beads were resuspended in the vial, then the appropriate volume (50 μl per sample) was transferred to a microfuge tube. The same volume of Washing Buffer (at least 1 ml 1×PBS with 0.1% BSA and 2 mM EDTA) was added and the bead sample was resuspended. The tube was then placed into a magnetic rack for 1-2 min and the supernatant was discarded.

The tube was removed from the magnetic rack and the beads were washed once. The washed beads were resuspended in the same volume of 1×IP buffer (50 mM sodium phosphate pH 7.0, 700 mM NaCl, 0.25% Triton X-100) as the initial volume of beads. Beads (50 μl) were added to the 500 μl of DNA-antibody mixture from the overnight incubation, then incubated for 2 hours on a rotator at 4° C. After the incubation, the bead-antibody-DNA complex was washed three times with 1×IP buffer as follows: The tube was placed into a magnetic rack for 1-2 min and the supernatant was discarded, then the magnetic bead antibody pellet was washed with 1×IP buffer 3 times.

The washed bead antibody DNA pellet was then resuspended in 250 μl digestion buffer with 3.5 μl Proteinase K (20 mg/ml). The sample was incubated for 2-3 hours on a rotator at 55° C., then 250 μl of buffered Phenol-Chloroform-Isoamylalcohol solution was added to the sample, and the tube was vortexed for 30 sec and then centrifuged at 14,000×g for 5 min at room temperature. The aqueous supernatant was carefully removed and transferred to a fresh microfuge tube. Then 250 μl chloroform were added to the supernatant from the previous step, vortexed for 30 sec and centrifuged at 13,500×g for 5 min at room temperature. The aqueous supernatant was removed and transferred to a fresh microfuge tube. To the supernatant 2 μl of glycoblue (20 mg/ml), 20 μl of 5M NaCl and 500 μl ethanol were added and mixed well, then precipitated in −20° C. freezer for 1 hour to overnight.

The precipitate was centrifuged at 13,500×g for 20 min at 4° C. and the supernatant was removed, while not disturbing the pellet. The pellet was washed with 500 μl cold 70% ethanol in −20° C. freezer for 15 min then centrifuged again at 13,500×g for 5 min at 4° C. and the supernatant was discarded. The tube was spun again briefly to collect residual ethanol to the bottom of the tube and as much liquid as possible was removed with gel loading tip. The pellet was air-dried at RT until it looked dry (about 5 min) then resuspended in 20 μl H₂O or TE. DNA concentration was measured in Qubit (Life Technologies) with ssDNA kit (Molecular Probes Q10212).

### MeDIP-Seq Analysis

The MeDIP DNA samples (50 ng of each) were used to create libraries for next generation sequencing (NGS) using the NEBNext Ultra RNA Library Prep Kit for Illumina (San Diego, CA) starting at step 1.4 of the manufacturer's protocol to generate double stranded DNA. After this step the manufacturer's protocol was followed. Each sample received a separate index primer. NGS was performed at WSU Spokane Genomics Core using the Illumina HiSeq 2500 with a PE50 application, with a read size of approximately 50 bp and approximately 25 (10-50 range) million reads per sample, and 6-7 sample libraries each were run in one lane.

### Molecular Bioinformatics and Statistics

Basic read quality was verified using information produced by the FastQC program. Reads were filtered and trimmed to remove low quality base pairs using Trimmomatic. The reads for each sample were mapped to the GRCh38 human genome using Bowtie2 with default parameter options. The mapped read files were then converted to sorted BAM files using SAMtools. To identify DMR, the reference genome was broken into 1000 bp windows.

The MEDIPS R package was used to calculate differential coverage between control and exposure sample groups. The EdgeR p-value was used to determine the relative difference between the two groups for each genomic window. Windows with an EdgeR p-value less than 10⁻⁴ were considered DMRs. The DMR edges were extended until no genomic window with an EdgeR p-value less than 0.1 remained within 1000 bp of the DMR. CpG density and other information was then calculated for the DMR based on the reference genome.

DMR were annotated using the biomaRt R package to access the Ensembl database. The genes that overlapped with DMR were then input into the KEGG pathway search to identify associated pathways. The DMR associated genes were then sorted into functional groups using information provided by the DAVID and Panther databases incorporated into a curated database.

### Example 2

Analysis of DMRs from Samples

For the instant example, samples were obtained from females of similar age and race. The white Caucasian non-Hispanic female samples were obtained by the Arthritis Northwest (ANW) Clinic in Spokane, Washington. The African American (AA) female samples were obtained by Dx Biosamples, LLC in San Diego, California, with sample collections in the Los Angeles California area. A comparison of the individuals, categorized as either having rheumatoid arthritis or not having rheumatoid arthritis and including information for age, race, and clinical information, is shown in Table 6. Table 6 shows the subject number (subject identification), the group (control or rheumatoid arthritics) the age of the subject, their race, their gender and rheumatoid arthritis diagnostic information.

| | | | | | |
|---|---|---|---|---|---|
| Subject | | | | | RA Diagnostic |
| # | Group | Age | Race | Gender | Information |

(A) Caucasian Population Samples

| ANWC01 | Control | 74 | Caucasian | F | Not Applicable |
|---|---|---|---|---|---|
| ANWC02 | Control | 56 | Caucasian | F | Not Applicable |
| ANWC03 | Control | 39 | Not | F | Not Applicable |
| | | | specified | | |
| ANWC04 | Control | 56 | Caucasian | F | Not Applicable |
| ANWC05 | Control | 59 | Caucasian | F | Not Applicable |
| ANWC06 | Control | 68 | Caucasian | F | Not Applicable |
| ANWC07 | Control | 55 | Caucasian | F | Not Applicable |
| ANWC08 | Control | 70 | Caucasian | F | Not Applicable |
| ANWC09 | Control | 49 | Caucasian | F | Not Applicable |
| ANWC10 | Control | 62 | Caucasian | F | Not Applicable |
| ANWC11 | Control | 30 | Caucasian | F | Not Applicable |
| ANWC12 | Control | 51 | Caucasian | F | Not Applicable |
| ANWC13 | Control | 45 | Caucasian | F | Not Applicable |
| Mean age ± SEM | | 54.9 ± | | | |
| | | 3.4 | | | |
| ANWT01 | Rheumatoid | 73 | Caucasian | F | High RA |
| | arthritis | | | | activity, RF+, |
| | | | | | CCP+, CDAI+ |
| ANWT03 | Rheumatoid | 61 | Caucasian | F | High RA |
| | arthritis | | | | activity, RF+, |
| | | | | | CCP+, CDAI+ |
| ANWT04 | Rheumatoid | 41 | Caucasian | F | High RA |
| | arthritis | | | | activity, RF+, |
| | | | | | CCP+, CDAI+ |
| ANWT05 | Rheumatoid | 56 | Caucasian | F | High RA |
| | arthritis | | | | activity, RF+, |
| | | | | | CCP+, CDAI+ |
| ANWT06 | Rheumatoid | 57 | Caucasian | F | High RA |
| | arthritis | | | | activity, RF+, |
| | | | | | CCP+, CDAI+ |
| ANWT09 | Rheumatoid | 66 | Caucasian | F | High RA |
| | arthritis | | | | activity, RF+, |
| | | | | | CCP+, CDAI+ |
| ANWT10 | Rheumatoid | 50 | Caucasian | F | Moderate RA |
| | arthritis | | | | activity, RF+, |
| | | | | | CDAI+ |
| ANWT12 | Rheumatoid | 74 | Caucasian | F | Moderate RA, |
| | arthritis | | | | activity RF+, |
| | | | | | CDAI+ |
| ANWT13 | Rheumatoid | 54 | Caucasian | F | High RA |
| | arthritis | | | | activity, RF+, |
| | | | | | CCP+, CDAI+ |
| ANWT14 | Rheumatoid | 68 | Caucasian | F | High RA |
| | arthritis | | | | activity, RF+, |
| | | | | | CCP+, CDAI+ |
| ANWT15 | Rheumatoid | 26 | Caucasian | F | High RA |
| | arthritis | | | | activity, RF+, |
| | | | | | CCP+, CDAI+ |
| ANWT16 | Rheumatoid | 48 | Caucasian | F | High RA |
| | arthritis | | | | activity, RF+, |
| | | | | | CCP+, CDAI+ |
| ANWT17 | Rheumatoid | 48 | Native | F | High RA |
| | arthritis | | American | | activity, RF+, |
| | | | | | CCP+, CDAI+ |

| | |
|---|---|
| Mean age ± SEM | 55.5 ± |
| | 3.7 |

(B) African-American Population Samples

| AH_10 | Control | 55 | African | F | Not Applicable |
|---|---|---|---|---|---|
| | | | American | | |
| AH_12 | Control | 52 | African | F | Not Applicable |
| | | | American | | |
| AH_13 | Control | 55 | African | F | Not Applicable |
| | | | American | | |
| AH_14 | Control | 57 | African | F | Not Applicable |
| | | | American | | |
| AH_15 | Control | 52 | African | F | Not Applicable |
| | | | American | | |
| AH_16 | Control | 58 | African | F | Not Applicable |
| | | | American | | |
| AH_17 | Control | 65 | African | F | Not Applicable |
| | | | American | | |
| AH_18 | Control | 61 | African | F | Not Applicable |
| | | | American | | |
| AH_19 | Control | 57 | African | F | Not Applicable |
| | | | American | | |
| Mean age ± SEM | | 56.8 ± | | | |
| | | 1.3 | | | |
| AH_20 | Rheumatoid | 55 | African | F | High RA |
| | arthritis | | American | | activity, |
| | | | | | CDAI+ |
| AH_22 | Rheumatoid | 51 | African | F | High RA |
| | arthritis | | American | | activity, |
| | | | | | CDAI+ |
| AH_23 | Rheumatoid | 53 | African | F | High RA |
| | arthritis | | American | | activity, |
| | | | | | CDAI+ |
| AH_24 | Rheumatoid | 56 | African | F | Moderate RA |
| | arthritis | | American | | activity, |
| | | | | | CDAI+ |
| AH_25 | Rheumatoid | 58 | African | F | High RA |
| | arthritis | | American | | activity, |
| | | | | | CDAI+ |
| AH_26 | Rheumatoid | 52 | African | F | High RA |
| | arthritis | | American | | activity, |
| | | | | | CDAI+ |
| AH_27 | Rheumatoid | 53 | African | F | Moderate RA |
| | arthritis | | American | | activity, |
| | | | | | CDAI+ |
| AH_28 | Rheumatoid | 50 | African | F | High RA |
| | arthritis | | American | | activity, |
| | | | | | CDAI+ |
| AH_29 | Rheumatoid | 51 | African | F | High RA |
| | arthritis | | American | | activity, |
| | | | | | CDAI+ |
| AH_30 | Rheumatoid | 50 | African | F | High RA |
| | arthritis | | American | | activity, |
| | | | | | CDAI+ |
| AH_31 | Rheumatoid | 52 | African | F | High RA |
| | arthritis | | American | | activity, |
| | | | | | CDAI+ |
| AH_32 | Rheumatoid | 52 | African | F | High RA |
| | arthritis | | American | | activity, |
| | | | | | CDAI+ |
| AH_33 | Rheumatoid | 63 | African | F | High RA |
| | arthritis | | American | | activity, |
| | | | | | CDAI+ |

| | |
|---|---|
| Mean age ± SEM | 53.5 ± |
| | 1.0 |

Upon collection of buccal cell swabs, samples were frozen at −20° C. then shipped on dry ice and stored at −80° C. until use. For Caucasian patients, a blood sample was also obtained and shipped immediately on ice for isolation of monocytes. Monocytes were purified with an antibody bead procedure. After isolation, the monocytes were stored at −80° C. prior to use.

The Caucasian (CC) buccal cell control groups without RA (n=13) and with RA (n=13) were compared. The Caucasian monocyte cell control groups without RA (n=13) and with RA (n=13) were compared. The African American (AA) buccal cell control groups without RA (n=9) and with RA (n=13) were compared. A combination of Caucasian and African American samples without (n=23) and with RA (n=26) were also compared.

Table 6 provides clinical information and RA diagnostic information. The Caucasian samples had RA diagnostic assays of Rheumatoid Factor autoantibody (RF) and Citrullinated Peptide autoantibody (CCP), and more qualitative RA activity assays of Clinical Disease Activity Index (CDAI) with DAS28 and RAPID3 supportive analysis.

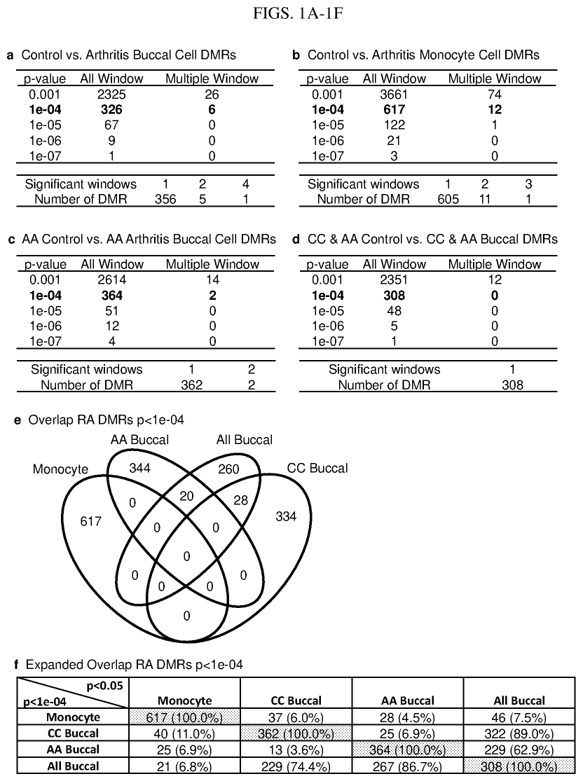

The DNA was extracted from each sample, then sonicated to 150-300 bp fragments and used for a methylated DNA immunoprecipitation (MeDIP) protocol. This involved an antibody to 5-methylcytosine and a magnetic bead procedure. The immunoprecipitated methylated DNA was then used to generate a sequencing library for an MeDIP-Seq analysis. MeDIP-Seq allows for greater than 90% of the genome to be examined for this EWAS analysis. Each sample for the MeDIP-Seq analysis had approximately 25 million reads. The EdgeR statistical analysis was used to identify differential DNA methylation regions (DMRs). Various p-value thresholds are presented for each of the comparisons, and p<1e-04 was selected for subsequent analysis as shown in FIGS. 1A-1F.

The majority of DMRs had one significant 1 kb window, but some had multiple windows. The Caucasian (CC) control versus RA buccal had 362 DMRs (see FIG. 1A). The CC control versus RA monocytes had 617 DMRs (see FIG. 1B). The AA control versus AA RA buccal had 364 DMRs (see FIG. 1C). The combination of CC and AA RA buccal samples had 308 DMRs (see FIG. 1D).

A Venn diagram of the different comparison DMRs at p<1e-04 had no overlap, except for the all buccal combined CC and AA that had approximately a 10% overlap (see FIG. 1E). An extended overlap of the p<1e-04 DMR comparisons with the others at a reduced statistical threshold of p<0.05 was used to determine if a DMR overlap is present at a reduced threshold (see FIG. 1F). From the horizontal row, the same cell type has 100% overlap. The same row allows potential overlaps at a reduced statistical threshold to be identified. The overlap was 11% between the Caucasian monocyte and buccal cell and other overlaps were between 3-7% (see FIG. 1F).

The combined CC and AA buccal sample analysis had a 74% overlap with the CC buccal and 87% with the AA buccal (see FIG. 1F), suggesting a preferred RA biomarker set using the combined analysis DMRs.

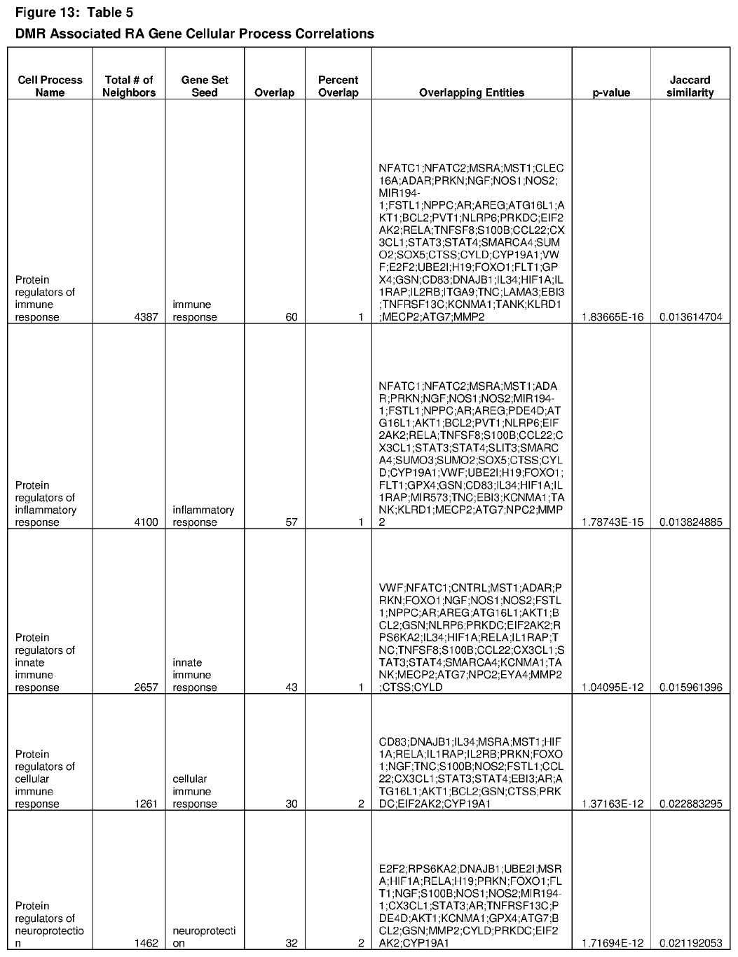

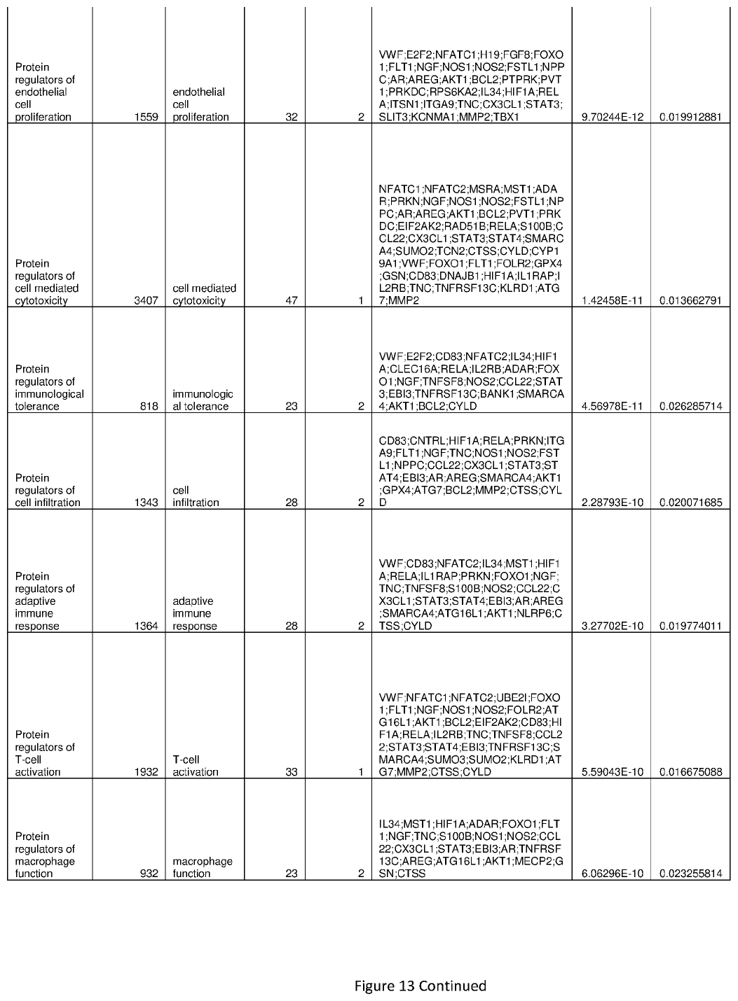

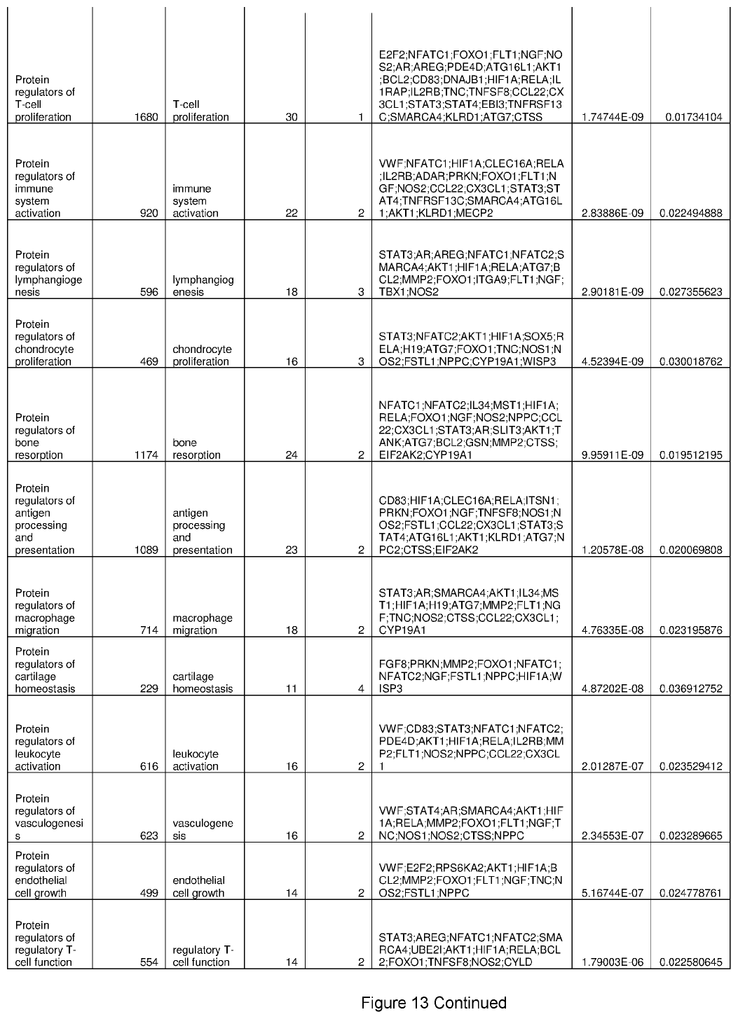

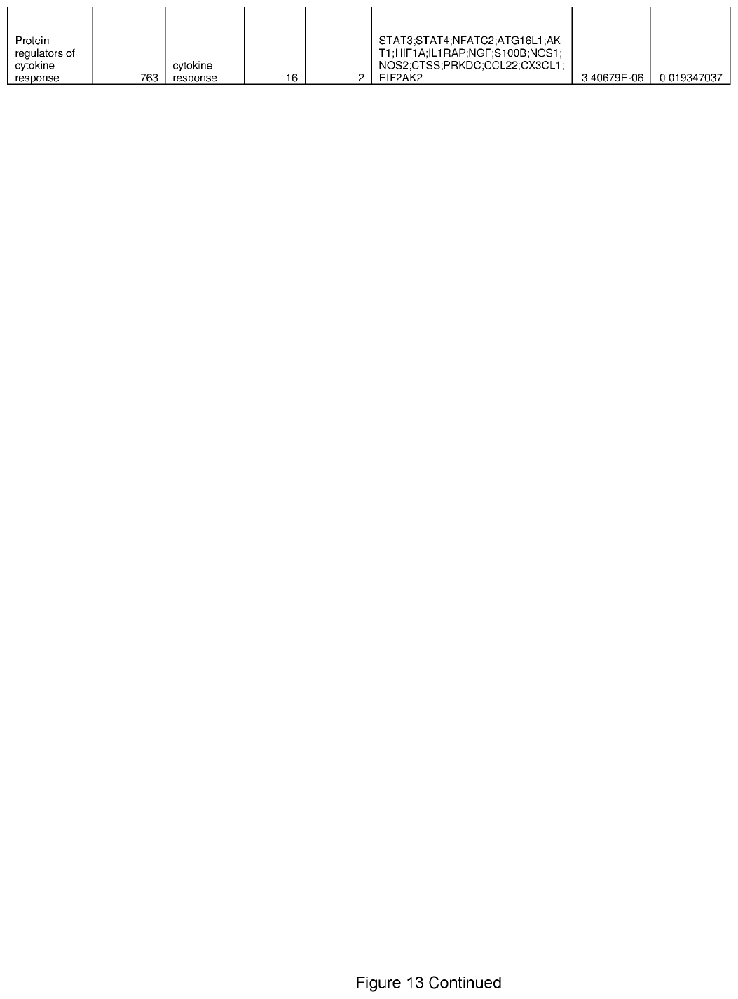

Tables 1-4 (presented in FIGS. 9-13, respectively) provide information for each comparison set of DMRs, including the DMR name, chromosomal location, start and stop nucleotide sites on the chromosome, length of the region in basepairs (bp), number of 1 kb significant windows, EdgeR p-value, maximum log-fold change (maxLFC) (i.e., positive values being an increase in DNA methylation, and negative values a decrease in DNA methylation), CpG number and density, and DMR associated gene(s) and gene category.

### Example 3

### Chromosomal Location Analysis of DMRs

The chromosomal locations of the DMRs are presented in FIGS. 2A-2D with chromosome number and location (megabase) indicated for each with an arrowhead. The black boxes indicate a cluster of DMRs. All the chromosomes contain DMRs with this genome-wide analysis.

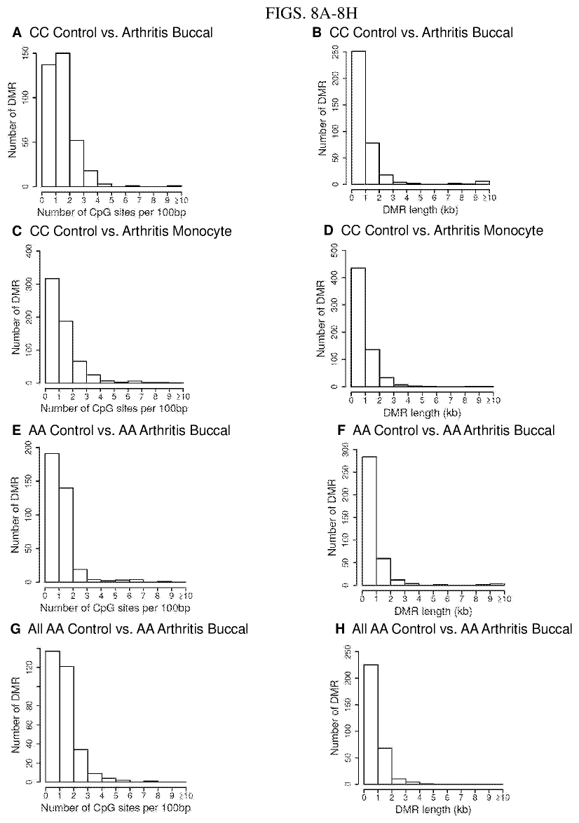

This example demonstrates that the RA DMRs (e.g., epimutations) are observed to be genome-wide. The DMR characteristics demonstrate a low CpG density, termed a CpG desert, of 1-3 CpG/100 bp for all the comparison DMRs (see FIGS. 8A-8H). The sizes of the DMRs are predominantly 1 kb in size with some being 2-4 kb in size for all the comparisons.

### Example 4

### Principal Component Analysis (PCA) of DMRs

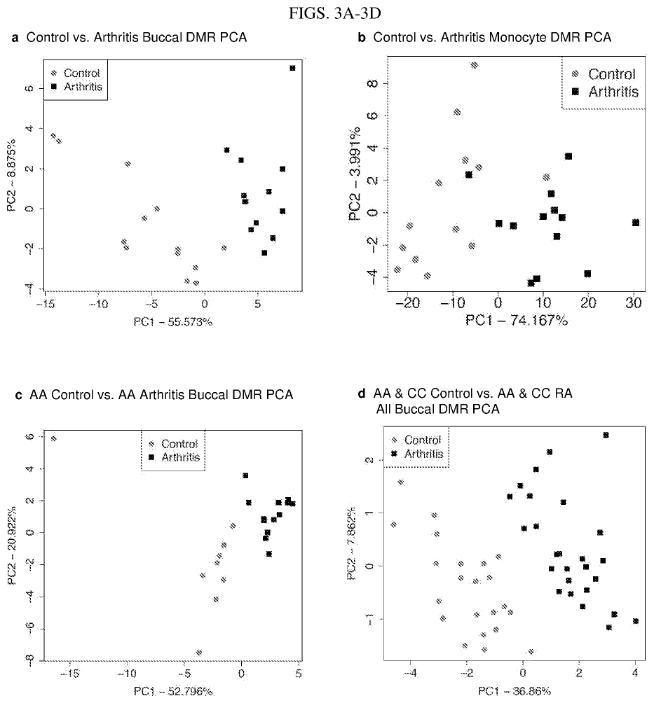

A comparison of the principal components in a Principal Component Analysis (PCA) demonstrates that the DMR components for the control versus RA cluster separately for each of the comparison groups (see FIGS. 3A-3D). The only exception observed was with the Caucasian control versus RA monocytes that showed one control and one RA each that overlap with the PCA cluster (see FIG. 3B). The overlapping RA group DMR at PC1=−8 and PC2=2.2 was 74 year age. The other individuals at >70 year age were the DMRs at PC1=−4 and PCR=−18, PC1=31 and PC2=−0.5, and PC1=−4 and PC2=9.2 (FIG. 3B). Higher variability was observed with the >70 year individuals.

Therefore, the PCA indicates principal components of the DMRs are predominantly distinct between the control and arthritis groups. In addition, the Native American and individual not specified for race (Table 6) were shown to not be outliers with the PCA analysis (FIGS. 3A-3D) and were included in the analysis. The Caucasian sample sets had a few 70 year and 30 year outliers compared to the mean of approximately 55 year samples (see Table 6). The significance of each DMR is indicated with minimum p-value and minimum FDR as shown in Tables 1-4.

### Example 5

### Analysis of DMR Associated Gene Categories

Less than half of the RA DMRs identified with each comparison had DMR associated genes within 10 kb of the DMR (see Tables 1-4). The 10 kb distance was used to include the proximal and distal promoter regions of the genes. The RA DMR associated gene categories were identified and are presented in FIG. 4A. The DMR numbers for each associated gene functional category are presented for each comparison. The signaling, transcription, metabolism and receptor are predominant for each of the DMR comparisons. This does reflect the major gene categories within the human genome with metabolism, transcription and signaling being the largest gene categories. This gene category analysis was for an individual DMR gene association analysis and not to reflect group combined function.

In contrast, a gene pathway analysis (KEGG) was performed, and those pathways in two or more different comparison DMR associated genes with similar function were identified in FIG. 4B. The metabolic pathway was excluded as it is present in all comparisons and over-represented in pathway analysis. The common signaling pathways include pathways in cancer, pathways in neurodegenerative disease, and specific pathways such as P13K-Akt (see FIG. 4B).

### Example 6

Correlation of RA DMR Associated Genes with Pathologies and Processes

The instant example correlated the RA DMR associated genes with pathologies and processes. Pathway Studio was used to identify the associated pathologies.

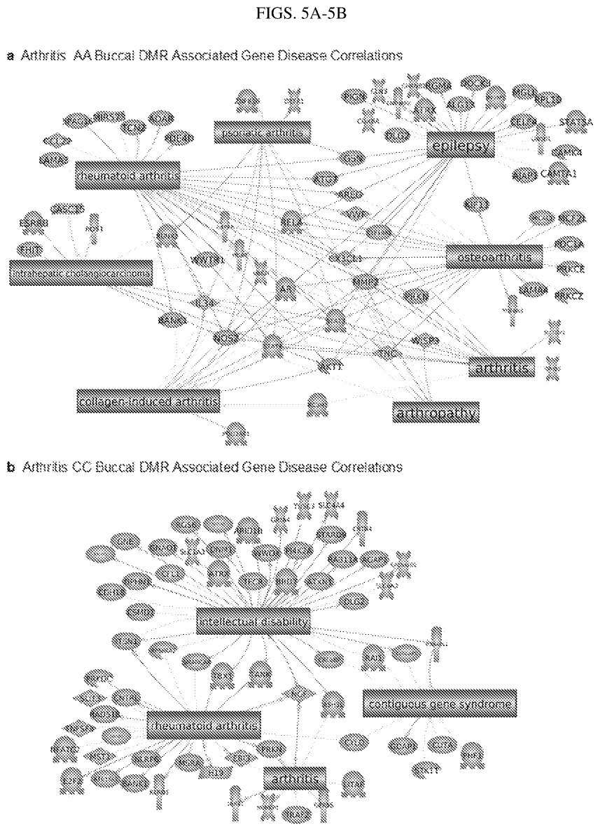

The African American RA buccal cell DMR associated gene correlations to diseases are presented in FIG. 5A. Six of the major disease correlations were arthritis related and the others are cancer and neurodegenerative related. Therefore, FIG. 5A shows the predominant African American RA DMRs in buccal cells are associated with arthritis pathologies.

The Caucasian buccal cell RA DMR associated gene correlations with rheumatoid arthritis and arthritis pathologies are shown in FIG. 5B, with contiguous gene syndrome and intellectual disability as additional pathologies significantly correlated.

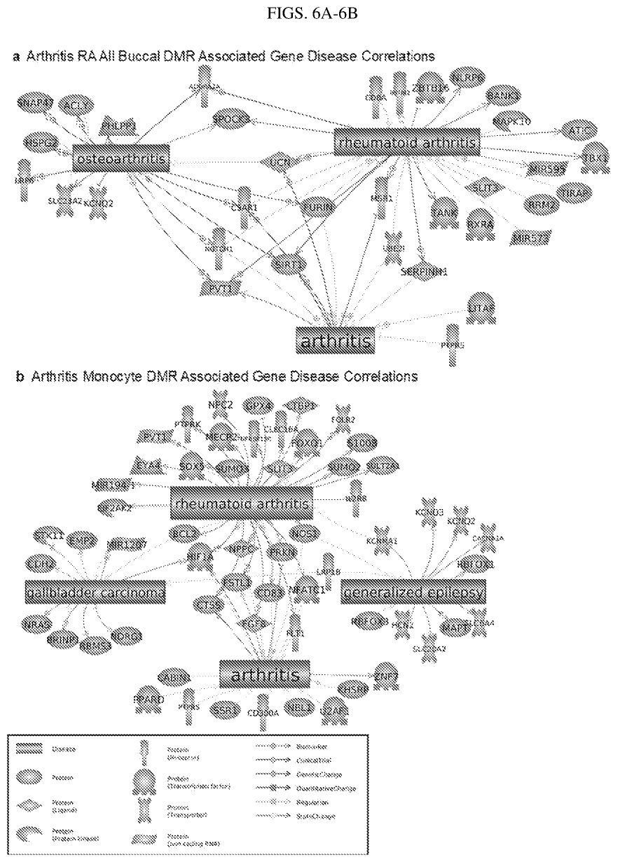

The combination CC and AA analysis also provided DMR associated genes and arthritis gene associations (see FIG. 6A). The Caucasian monocyte DMRs also predominantly associated with arthritis and RA correlated genes, with additional epilepsy and carcinoma associated genes (see FIG. 6B).

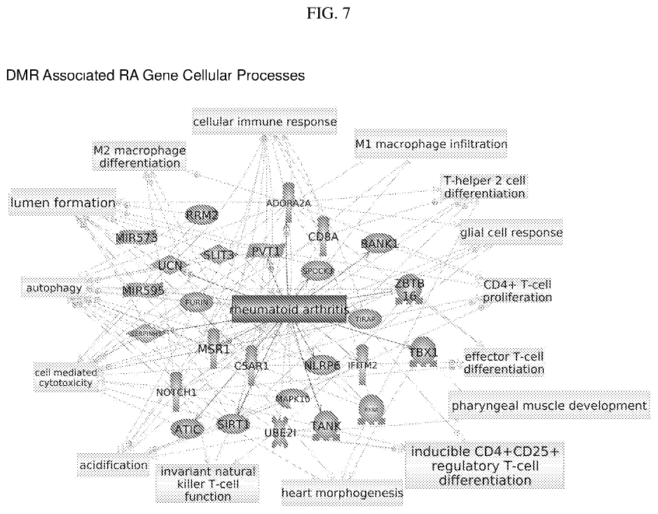

Observations demonstrate the RA DMRs identified with all the comparisons showed significant connections to genes correlated to arthritis pathologies, including rheumatoid arthritis. An additional analysis used the DMR associated genes for RA from each of the comparisons to identify known RA cellular process correlations as shown in FIG. 7. Previous rodent and human studies have identified known gene correlations with RA associated cell processes, and these same genes and correlations were observed in the current study (see FIG. 7 and Table 5). Over 25 different RA gene cell processes were identified with a statistical significance p<1e-06, with the four most significant being protein regulators of immune response, inflammatory response, innate immune response, and cellular immune response (see Table 5). These DMR associated rheumatoid arthritis gene cell process correlations are shown in FIG. 7 and Table 5.

Therefore, the RA DMR associated genes identified in the present disclosure correlated to previously identified RA associated genes and cellular processes, as well as identified potential new RA associated genes.

1. A method comprising:
obtaining a sample of a somatic cell from a human subject;
isolating deoxyribonucleic acid (DNA) from the sample, thereby obtaining isolated DNA; detecting and determining a methylation level of a differential DNA methylation region (DMR) comprised in the isolated DNA; and
comparing the methylation level of the DMR to a reference methylation level of a corresponding reference DMR;
wherein the comparing comprises employing a computer comprising a computer processor and computer readable memory comprising computer readable instructions contained thereon,
wherein the detecting and determining comprises a methylated DNA immunoprecipitation (MeDIP), a sequencing, a bisulfite treatment, a bisulfite conversion, a deamination of an unmethylated cytosine base, employing an array, or any combination thereof, and
wherein a plurality of distinct DMRs are detected and compared, wherein the plurality comprises at least 10 distinct DMRs.

2. The method of claim 1, wherein the somatic cell comprises a buccal cell.

3. The method of claim 1, wherein the somatic cell comprises a blood monocyte cell, and optionally wherein the blood monocyte cell is a purified blood monocyte cell.

4. (canceled)

5. (canceled)

6. (canceled)

7. (canceled)

8. The method of claim 1, wherein the plurality of distinct DMRs comprises about 10 to about 15 DMRs, about 50 to about 100 distinct DMRs, or about 100 to about 150 distinct DMRs.

9. The method of claim 1, wherein the plurality of distinct DMRs comprises about 150 to about 200 distinct DMRs or about 200 to about 250 distinct DMRs.

10. (canceled)

11. The method of claim 1, wherein the detected DMRs comprise DMRs from at least about 1, 2, 3, 4, 5, 6, 7, 8, 9, 10, 11, 12, 13, 14, 15, 16, 17, 18 19, 20, 21, 22, or 23 chromosomes; or wherein the detected DMRs are DMRs are from at least about 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23 chromosomes.

12. (canceled)

13. The method of claim 1, wherein at least a plurality of the DMRs that are determined and compared comprise a CpG density of 1 to about 10 CpG per 100 nucleotides.

14. The method of claim 13, wherein at least a plurality of the DMRs that are determined and compared comprise a CpG density of 1 to about 3 CpG per 100 nucleotides.

15. The method of claim 1, wherein at least about 50, 60, or 70 percent of the DMRs that are determined and compared are hypermethylated when compared, individually, to individual reference methylation levels of corresponding individual reference DMRs.

16. (canceled)

17. The method of claim 1, wherein the human subject is a Caucasian subject.

18. The method of claim 1, wherein the human subject is an African-American subject.

19. The method of claim 1, wherein the method further comprises determining a risk of the human subject having a disease or condition, wherein the determining is performed with a computer comprising a computer processor and computer readable memory comprising computer readable instructions.

20. The method of claim 19, wherein the disease or condition is rheumatoid arthritis.

21. The method of claim 19, wherein the risk of the human subject further comprises consideration of smoking status of the human subject, nutritional status of the human subject, alcohol consumption of the human subject, or any combination thereof.

22. (canceled)

23. (canceled)

24. The method of claim 1, further comprising treating the human subject.

25. The method of claim 24, wherein the treating comprises administering a medication for treatment of rheumatoid arthritis.

26. The method of claim 25, wherein the medication is an antibody.

27. The method of claim 25, wherein the medication is an anti-inflammatory.

28. The method of claim 24, wherein the treating comprises administering a medication for preventative treatment of rheumatoid arthritis.

29. The method of claim 1, further comprising transmitting data, a result, or both, via an electronic communication medium.

30-83. (canceled)
